SEQUENCE LISTING (41 sequences)

SEQ ID NO: 1 - DNA, 885 bp, Pseudomonas koreensis
  source 1..885: mol_type = other DNA
atgcgccccgaaattgccgtgctcgacatccagggccagtaccgggtctataccgagttctaccgtgccgatgccgcggagaagaccatcatcctggtcaacggctccatggccactaccgcctcgttcgcccagaccgtgaagaacctgcatccccagttcaacgtcgtgtgctacgatcagccctacgctggccgcagcaagatccacaaccgccatgaaaagcatctgaccaaggaagtcgagggccagatcctgctggagctgatcgatcacttcggtgccgagcatgtgctgtcgttcagctggggcggcgcggcaacgctggtggcgctgtcgcaccagccccgccgcatcgaaaaggcggtgatctcgtccttcagcccggtgatcaatgcgcacatgctcgactacctggagcgcggcgttgactacctgggccagcgcgacggcgatcgtgtcggccatctggtcaacaataccatcggcaagcacctaccgagcctgttcaaacgcttcaactaccgtcacgtgtccagcctggccgagcatgaatacggccagatgcacttccatatcaacgatgtgctccacagcgatcgccagtgctacctgaacgccgccaagaagatcaacgtgccggtgctgtttttgaacggcgagtgggacgaatataccgccgccgaggatgcgcgcatcttcggcaaccacgtcgcccagtcgacctttaccacgctccaggcaaccggccacttcctcgacatggagcacaaggccgcctgccgcgacagccagaccgcgctgctcggcttcctcaagccgtcgcaacagtccagccgcacgcgctactcgttcgtccaggatcaccatgcgctggccatttaa

SEQ ID NO: 2 - protein, 294 aa, Pseudomonas koreensis
MRPEIAVLDIQGQYRVYTEFYRADAAEKTIILVNGSMATTASFAQTVKNLHPQFNVVCYDQPYAGRSKIHNRHEKHLTKEVEGQILLELIDHFGAEHVLSFSWGGAATLVALSHQPRRIEKAVISSFSPVINAHMLDYLERGVDYLGQRDGDRVGHLVNNTIGKHLPSLFKRFNYRHVSSLAEHEYGQMHFHINDVLHSDRQCYLNAAKKINVPVLFLNGEWDEYTAAEDARIFGNHVAQSTFTTLQATGHFLDMEHKAACRDSQTALLGFLKPSQQSSRTRYSFVQDHHALAI

SEQ ID NO: 3 - protein, 295 aa, Pseudomonas putida
MRPEIAVLDIQGQYRVYTEFYRADAAENTIILINGSLATTASFAQTVRNLHPQFNVVLFDQPYSGKSKPHNRQERLISKETEAHILLELIEHFQADHVMSFSWGGASTLLALAHQPRYVKKAVVSSFSPVINEPMRDYLDRGCQYLAACDRYQVGNLVNDTIGKHLPSLFKRFNYRHVSSLDSHEYAQMHFHINQVLEHDLERALQGARNINIPVLFINGERDEYTTVEDARQFSKHVGRSQFSVIRDAGHFLDMENKTACENTRNVMLGFLKPTVREPRQRYQPVQQGQHAFAI

SEQ ID NO: 4 - protein, 295 aa, Pseudomonas oleovorans
MRPEIAVLDIQGQYRVYTEFYRADAAENTIILINGSLATTASFAQTVRNLHPQFNVVLFDQPYSGKSKPHNRQERLISKETEAHILLELIEHFQADHVMSFSWGGASTLLALAHQPRYVKKAVVSSFSPVINEPMRVYLDRGCQYLAACDHYQVGNLVNDTIGKHLPSLFKRFNYRHVSSLDSHEYAQMHFHINQVLEHDLERALQGARNINIPVLFINGERDEYTTVEDARQFSKHVGRSQFSVIRDAGHSLDMENKTACENTRNVMLGFLKPTVREPRQRYQPVQQGQHAFAI

SEQ ID NO: 5 - protein, 285 aa, Pseudomonas aeruginosa
MPTAWLDLPAPPALPGLFLRAALRRGIRGKALPERGLRSQVTVDPKHLERYRQVCGFRDDGLLPPTYPHILAFPLQMALLTDKRFPFPLLGLVHLENRIDVLRALGGLGPFTVSVAVENLQPHDKGATFSIVTRLEDQLGLLWVGDSKVLCRGVKVPGEIPPKAEQEPLPLEPVDNWKAPADIGRRYARAAGDYNPIHLSAPSAKLFGFPRAIAHGLWNKARSLAALGERLPASGYRVEVRFQKPVLLPASLTLLASAAAADGQFSLRGKDDLPHMAGHWSRLQG

SEQ ID NO: 6 - DNA, 1194 bp, Treponema denticola
  source 1..1194: mol_type = other DNA
atgattgtcaaaccgatggtgcgcaacaatatctgcctcaatgcgcatccgcaaggctgcaagaagggcgtcgaggatcagatcgagtataccaagaagcgcattaccgccgaggtgaaggccggtgccaaggcaccgaagaacgtgctggtgctgggctgctccaacggctacgggctggcttccaggattaccgcggccttcggctacggtgcggcgaccatcggcgtgagcttcgagaaggcaggctcggaaacgaagtacggcacgccgggctggtacaacaacctggccttcgacgaggccgccaagcgcgaggggctgtacagcgtgaccattgacggcgacgccttcagcgacgagatcaaggcgcaagtgatcgaagaggccaagaagaagggcatcaagttcgatctgatcgtctattcgctggccagcccggtgcgtaccgatcccgataccggcatcatgcacaagagcgtgctgaagccgttcggcaagacctttaccggcaagaccgtcgatccgtttaccggcgaactcaaggagatctcggccgagcccgccaacgacgaagaagccgccgcgaccgtcaaagtgatgggcggcgaggactgggaacgctggatcaaacagctctccaaggaagggctgctggaagagggctgcatcacgctggcctactcgtacatcggccccgaggcgacccaggcgctctaccgcaagggtaccatcggcaaggccaaggaacatctggaagcaaccgcgcatcgcctgaacaaggagaacccgtccatccgtgccttcgtgtcggtcaacaagggcctggttacccgtgcctccgccgtgatcccggtgattccgctgtacctggcctcgctgttcaaggtcatgaaggagaagggcaaccacgaaggctgcatcgaacagatcacgcgcctgtacgccgaacgcctgtaccgcaaggacgggaccatcccggtcgacgaagagaaccggatccgcatcgacgactgggaactggaagaggacgtccagaaggccgtgagcgcactgatggagaaggtgaccggcgagaatgccgaatcgctgaccgatctggcgggctaccgccacgatttcctggccagcaacggcttcgacgtggaaggcatcaactacgaggccgaagtcgagcgcttcgatcgcatttaa

SEQ ID NO: 7 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 8 - DNA, 1986 bp, Marinobacter hydrocarbonoclasticus
  source 1..1986: mol_type = other DNA
atgaactatttcctgactggcggcaccggctttatcggccgcttcctggtcgagaagctgctcgcgcgcggcggcacggtctatgtgctggtgcgcgaacaaagccaggacaagttggagcgcctgcgcgagcgctggggcgccgacgacaaacaggtcaaggccgtgatcggcgatctgacctcgaagaacctgggcatcgacgccaagacgctcaagtcgctgaagggcaacatcgatcacgtgttccatctggctgccgtctatgacatgggcgccgacgaggaagcccaggccgcgaccaacatcgaaggtacccgcgccgcggtccaggccgccgaggcgatgggcgccaagcacttccaccatgtcagcagcattgcggccgccggcctgttcaagggcatcttccgcgaagacatgttcgaagaagccgagaagctggatcacccgtacctgcgaaccaagcacgaatcggaaaaggtggtgcgcgaagaatgcaaggtgcccttccgcatctacaggcccggcatggtgatcggtcacagcgaaaccggcgagatggacaaggtggacgggccctactacttcttcaagatgatccagaagatccgccatgcgctgccgcaatgggtgccgaccatcggcatcgagggcggccggctcaatatcgtcccggtcgatttcgtggtcgacgcactggaccatatcgcacatctggaaggcgaggacggcaactgcttccatctggtggattctgatccgtacaaggtcggcgagatcctgaacatcttctgcgaagccggacacgccccgcgcatgggcatgcgcatcgacagccgcatgttcggctttatcccgccgtttatccggcaatcgatcaagaacctgccgccggtcaagcgcattaccggcgcgctgctggacgacatgggcatcccgcccagcgtgatgtcgttcatcaactacccgacgcgcttcgatacccgagagctcgagcgcgtcctgaagggtactgacatcgaggtgccgcgcctgccgagctacgccccggtgatctgggactactgggagcgcaacctggaccctgatctgttcaaggaccgtaccctgaagggaaccgtcgagggcaaggtgtgcgtggtgaccggcgcaacgtcgggcatcggcctggcgaccgccgaaaagctggccgaggccggcgcgatcctggtgatcggtgcccgaaccaaggaaacgctcgacgaggtggccgcctcgctcgaggccaagggcggcaacgtgcacgcctaccagtgcgacttcagcgacatggacgactgcgaccgcttcgtcaagaccgtgctggacaaccatggccacgtcgacgtgctggtcaataatgcgggccgcagcatccgccgcagcctggcgctgtcgttcgaccgcttccacgacttcgagcgcacgatgcagctcaactacttcggctcggtgcgcctgatcatgggttttgcgcccgccatgcttgagcgccgccgcggccacgtggtcaacatcagctcgatcggcgtgctgaccaacgcaccgcgcttcagcgcctacgtgtcgtcgaagagcgcgctggacgccttctcgcgctgcgcggccgccgagtggtcggaccgcaacgtgacgttcacgaccatcaacatgccgctggtcaagacgccgatgatcgcgccgaccaagatctatgactccgtgccgacgctcacgccggatgaagccgcccagatggtggccgatgccatcgtctaccgcccgaagcgcattgcgacccggcttggcgtcttcgcacaggtgctgcacgcgctggccccgaagatgggcgaaatcatcatgaataccggctatcgcatgttcccggattcgccggccgccgcgggctccaagtccggcgagaagcccaaggtgagcacggaacaggtggccttcgccgccatcatgcgcggcatctactggtaa

SEQ ID NO: 9 - protein, 661 aa, Marinobacter hydrocarbonoclasticus
MNYFLTGGTGFIGRFLVEKLLARGGTVYVLVREQSQDKLERLRERWGADDKQVKAVIGDLTSKNLGIDAKTLKSLKGNIDHVFHLAAVYDMGADEEAQAATNIEGTRAAVQAAEAMGAKHFHHVSSIAAAGLFKGIFREDMFEEAEKLDHPYLRTKHESEKVVREECKVPFRIYRPGMVIGHSETGEMDKVDGPYYFFKMIQKIRHALPQWVPTIGIEGGRLNIVPVDFVVDALDHIAHLEGEDGNCFHLVDSDPYKVGEILNIFCEAGHAPRMGMRIDSRMFGFIPPFIRQSIKNLPPVKRITGALLDDMGIPPSVMSFINYPTRFDTRELERVLKGTDIEVPRLPSYAPVIWDYWERNLDPDLFKDRTLKGTVEGKVCVVTGATSGIGLATAEKLAEAGAILVIGARTKETLDEVAASLEAKGGNVHAYQCDFSDMDDCDRFVKTVLDNHGHVDVLVNNAGRSIRRSLALSFDRFHDFERTMQLNYFGSVRLIMGFAPAMLERRRGHVVNISSIGVLTNAPRFSAYVSSKSALDAFSRCAAAEWSDRNVTFTTINMPLVKTPMIAPTKIYDSVPTLTPDEAAQMVADAIVYRPKRIATRLGVFAQVLHALAPKMGEIIMNTGYRMFPDSPAAAGSKSGEKPKVSTEQVAFAAIMRGIYW

SEQ ID NO: 10 - DNA, 552 bp, Escherichia coli
  source 1..552: mol_type = other DNA
atggccgatacgctgctgatcctgggtgattcgctgtcggccggctaccgcatgagcgccagtgccgcctggcccgcgctgctcaacgacaaatggcagtccaagaccagcgtggtcaatgcgtcgatctcgggcgatacctcgcaacagggcctggcgcgcctgcccgcgctcttgaaacagcaccagccgcgctgggtgctggtcgagctgggcggcaacgacggcctgcgcggcttccagccgcaacagaccgaacagacgctgcgccagatcctccaggatgtcaaggccgccaatgccgaaccgctgctgatgcagatccggctgcccgccaactacggacgccgctacaacgaggccttctcggcgatctaccccaagctggcgaaggagttcgatgtgccgctgctgcccttcttcatggaagaggtttacctcaagccccagtggatgcaagacgacggcatccatccgaaccgcgacgcccagccatttatcgcggactggatggcaaaacagctccagccgctggtcaaccacgattcttaa

SEQ ID NO: 11 - protein, 183 aa, Escherichia coli
MADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 12 - DNA, 399 bp, Escherichia coli
  source 1..399: mol_type = other DNA
atgcagacccagataaaagtgcgcggctaccatctggacgtctaccagcatgtgaacaacgcgcgctacctggagttcctggaagaggcgcgctgggacggcctggagaacagcgacagcttccaatggatgaccgcccacaacatcgcctttgtggtggtgaatatcaacatcaactaccggcgcccggccgtgttgtcggatctgctgaccattaccagccagctgcaacagctgaacggcaagagcggcatcctgagccaggtgatcacgctggagcccgagggccaggtggtggccgatgcgctgattaccttcgtgtgcatcgatctgaagacccagaaggcgctggcgctggaaggcgagctgcgcgagaagctggaacagatggtcaaataa

SEQ ID NO: 13 - protein, 132 aa, Escherichia coli
MQTQIKVRGYHLDVYQHVNNARYLEFLEEARWDGLENSDSFQWMTAHNIAFVVVNININYRRPAVLSDLLTITSQLQQLNGKSGILSQVITLEPEGQVVADALITFVCIDLKTQKALALEGELREKLEQMVK

SEQ ID NO: 14 - protein, 394 aa, Cupriavidus necator
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 15 - protein, 393 aa, Clostridium kluyveri
MREVVIVSAVRTAIGSFGGTLKDVSAVDLGAIVIKEAVKRAGIKPEQVDEVIFGNVIQAGVGQSLARQSAVYAGLPVEVPAFTVNKLCGSGLRTVSLAASLISNGDADTIVVGGSENMSASPYLIPKARFGYRMGEAKIYDAMLHDGLIDSFNNYHMGITAENIAEKWGITREDQDKFALASQQKAEAAIKAGKFKDEIVPVTVKMKKKEVVFDTDEDPRFGTTIETLAKLKPAFKRDGTGTVTAGNSSGINDSSAALILMSADKAKELGVKPMAKYVDFASAGLDPAIMGYGPYYATKKVLAKTNLTIKDFDLIEANEAFAAQSIAVARDLEFDMSKVNVNGGAIALGHPVGCSGARILVTLLHEMQKRDAKKGLATLCIGGGQGTAVVVER

SEQ ID NO: 16 - protein, 729 aa, Escherichia coli
MLYKGDTLYLDWLEDGIAELVFDAPGSVNKLDTATVASLGEAIGVLEQQSDLKGLLLRSNKAAFIVGADITEFLSLFLVPEEQLSQWLHFANSVFNRLEDLPVPTIAAVNGYALGGGCECVLATDYRLATPDLRIGLPETKLGIMPGFGGSVRMPRMLGADSALEIIAAGKDVGADQALKIGLVDGVVKAEKLVEGAKAVLRQAINGDLDWKAKRQPKLEPLKLSKIEATMSFTIAKGMVAQTAGKHYPAPITAVKTIEAAARFGREEALNLENKSFVPLAHTNEARALVGIFLNDQYVKGKAKKLTKDVETPKQAAVLGAGIMGGGIAYQSAWKGVPVVMKDINDKSLTLGMTEAAKLLNKQLERGKIDGLKLAGVISTIHPTLDYAGFDRVDIVVEAVVENPKVKKAVLAETEQKVRQDTVLASNTSTIPISELANALERPENFCGMHFFNPVHRMPLVEIIRGEKSSDETIAKVVAWASKMGKTPIVVNDCPGFFVNRVLFPYFAGFSQLLRDGADFRKIDKVMEKQFGWPMGPAYLLDVVGIDTAHHAQAVMAAGFPQRMQKDYRDAIDALFDANRFGQKNGLGFWRYKEDSKGKPKKEEDAAVEDLLAEVSQPKRDFSEEEIIARMMIPMVNEVVRCLEEGIIATPAEADMALVYGLGFPPFHGGAFRWLDTLGSAKYLDMAQQYQHLGPLYEVPEGLRNKARHNEPYYPPVEPARPVGDLKTA

SEQ ID NO: 17 - protein, 282 aa, Clostridium kluyveri
MSIKSVAVLGSGTMSRGIVQAFAEAGIDVIIRGRTEGSIGKGLAAVKKAYDKKVSKGKISQEDADKIVGRVSTTTELEKLADCDLIIEAASEDMNIKKDYFGKLEEICKPETIFATNTSSLSITEVATATKRPDKFIGMHFFNPANVMKLVEIIRGMNTSQETFDIIKEASIKIGKTPVEVAEAPGFVVNKILVPMINEAVGILAEGIASAEDIDTAMKLGANHPMGPLALGDLIGLDVVLAVMDVLYSETGDSKYRAHTLLRKYVRAGWLGRKSGKGFFAY

SEQ ID NO: 18 - protein, 729 aa, Escherichia coli; identical to SEQ ID NO: 16

SEQ ID NO: 19 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 20 - protein, 397 aa, Treponema denticola; identical to SEQ ID NO: 7

SEQ ID NO: 21 - protein, 183 aa, Escherichia coli; identical to SEQ ID NO: 11

SEQ ID NO: 22 - protein, 132 aa, Escherichia coli; identical to SEQ ID NO: 13

SEQ ID NO: 23 - protein, 302 aa, Clostridium beijerinckii
MSKNFDELLSRLKEVPTKKVAVAVAQDEPVLEAIKEATENNIAQAILVGDKQQIHEIAKKINLDLSDYEIMDIKDPKKATLEAVKLVSSGHADMLMKGLVDTATFLRSVLNKEVGLRTGKLMSHVAVFDVEGWDRLLFLTDAAFNTYPEFKDKVGMINNAVVVAHACGIDVPRVAPICPVEVVNTSMQSTVDAALLAKMSDRGQIKGCVIDGPFALDNAISEEAAHHKGVTGSVAGKADILLLPNIEAANVMYKTLTYFSKSRNGGLLVGTSAPVILTSRADSFETKVNSIALAALVAARNK

SEQ ID NO: 24 - protein, 355 aa, Clostridium felsineum
MYRLLIINPGSTSTKIGIYDDEKEIFEKTLRHSAEEIEKYDTIFDQFEFRKEVILDALKEANIEVSSLNAIIGRGGLLKPIVSGTYGVNDAMLKDLKVGVQGQHASNLGGIIANEIAKEINVPAYIVDPVVVDELEEIARVSGMADIPRKSIFHALNQKAVARRYAKETKKKYEDLNLIVVHMGGGTSVGTHKAGRVVEVNNTLDGEGPFSPERSGGVPVGDLVRMCFSKKYTYEEVMKKINGKGGVVSYLNTIDFKAVVDKALEGDKKCGLIYEAFTYQVAKDIGKCATVLKGKVDAIILTGGIAYNPYVCDAIEDRVKFIAPVVRYGGEDELLALAEGGLRVLRGEEKAKEYK

SEQ ID NO: 25 - protein, 661 aa, Marinobacter hydrocarbonoclasticus; identical to SEQ ID NO: 9

SEQ ID NO: 26 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 27 - protein, 339 aa, Escherichia coli
MSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 28 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 29 - protein, 323 aa, Cupriavidus necator
MKTTFLDFEQPIAELEAKIEELRFVQDDSAVDISEEISRLAGKSQQLTKDIYANLTPWQVAQIARHPQRPYTLDYVREIFTDFHELHGDRTFADDLSIIGGLARFNGQACMVIGHQKGRDTKERAMRNFGMPKPEGYRKAKRLMELADKFGLPIFTFVDTPGAFPGIDAEERGQSEAIGHNLYVMAGLKVPLIATIIGEGGSGGALAIAVGDVVQMLQFATYAVISPEGCASILWKTAEKAPEAAEALGLTAHRLKALGLIDKIVSEPLGGAHRDYKGMAAMLKRSLAESLRQFQGMSVKELQARRYERLLAYGKFKETGTQD

SEQ ID NO: 30 - protein, 155 aa, Cupriavidus necator
MDLRKLKTLIDLVAESGISELEVTEGDGKVRIVKQPPQIVAAPMAMPQMQALPPAAAAAPAAGAVAPAAEPAAAQLPAGHVVTSPMVGTFYRAPSPGAAPFVNVGDSVKEGQTVCIIEAMKLLNEIECDKAGVIKEILVENGQAVEYGQPLFVIG

SEQ ID NO: 31 - protein, 450 aa, Cupriavidus necator
MFEKILIANRGEIALRIQRACRELGIKTVVVYSEADKEAKYVRLADEAVCIGPAPSPLSYLNMPAIISAAEVTDAQAIHPGYGFLSENADFAERVEKSGFVFIGPTADSIRLMGDKVSAKQAMIKAGVPCVPGSDGALPDDPKEILATARRVGYPVIIKAAGGGGGRGMRVVHTEAALINAVNMTREEAGRAFGNPEVYMEKFLENPRHVEIQILADQHRQAIWLGERDCSMQRRHQKVIEEAPAPHIPRRLIERIGDRCAEACKKMGYRGAGTFEFLYENNEFYFIEMNTRVQVEHPVTEMITGIDIVQEQIRIAFGEKLRLRQKDVQFRGHAIECRVNAEDPFKFTPSPGRITAWHMPGGPGVRVDSHAYDGYFVPPNYDSMIGKIITYGATREQAIARMRIALSEMVVDGIQTNVPLHRELMMDANFVEGGTSIHYLEHRLAERAKA

SEQ ID NO: 32 - protein, 290 aa, Cupriavidus necator
MSWLDKLLPPKIQQTDPSTRKGIPEGLWVKCPSCESTLYRTDVEANLHVCPKCDHHMRISARARLDGLLDAEGRYEIGQEIVPVDALKFKDSKKYPDRIKAAMDDTGETDAMVVMGGAIHTIPVVVSCFEFEFMGGSMGSVVGERFVRGAQAALEQKVPFICFTATGGARMQESLLSLLQMAKTTSMLNQLSAAKLPFISVLTDPTMGGVSASFAFLGDVVIAEPKALIGFAGPRVIEQTVREKLPEGFQRSEFLIQKGAIDMIVDRRKMRAELAQLLALLQKQPADAVA

SEQ ID NO: 33 - protein, 309 aa, Cupriavidus necator
MKFAFVFPGQGSQSVGMLNAFADNAVVRATVEEASAALGQDLGRLIAEGPAEELNLTTNTQPVMLTAAVAVYRAWLDAGGPAPALVAGHSLGEYSALVAAGVIPFADAVPLVRFRAQAMQEAVPVGEGAMAAILGLSDDDVRAACAEASAAGVVEAVNFNAPSQVVIAGHKAAVEKACEIAKGKGAKRALPLPVSAPFHSSLLKPASDRLRERMAGLAFSAPSIPLVNNVDVAIVNDPDAIKDALVRQAAAPVRWVECVQKMAAEGVTHVIECGPGKVLAGMTKRIDGNLTGGAIFDPASLQDTLALLK

SEQ ID NO: 34 - protein, 400 aa, Cupriavidus necator
MSPLLFSHFTATSCLGAGVDATLSALRAQRGGLAPCRFGGVELDTFVGEVPGLDTVALPPALAEFDCRNNRLAQLALEQDGFAARVREAAARYGAHRVGVFLGTSTAGVLQTELAYRQRDPASGALPPGFHYATTHNPYSLPAFLRQYLGLTGPAAAVSSACSSGAKVFSSARRMLEAGLIDVAVVGGVDSLCHTTLYGFNSLELLSDQPCRPYDVARNGISIGEGAAFGLLERVAGSAQGDAILLAGIGESSDAHHMSTPHPDGLGARLALEQALASAGVAPAQVGYVNLHGTATRSNDAAEAMAMAAVLPGTPCSSTKGATGHALGAAGALEAVICALALRHGLMPGGINTTQVDPALDVNYQLANREAPLRYAMTNAFGFGGSNCSLLFARADAASH

SEQ ID NO: 35 - protein, 410 aa, Cupriavidus necator
MSRRRVVVTGLGLVSPVGNTVAEGWANLVAGKSGIATITKFDHSALSVHFAGEVKGFNAEDYIPAKEARAMDTFIHFGIAAGTQALKDSGLEVTEANAERIGVLVGSGIGGLPLIEETHAVLTERGPRRISPFFVPGSIINMIAGHLSIIHGIKGPNLAAVTACTTGLHSIGLAARLIQAGDADAMLAGGAESTVSPLGIGGFAAARALSTRNDDPAAASRPWDKDRDGFVLGEGAGVMMLEEYESAKARGARIYAELIGFGMSGDAYHMTAPNMDGPRRCMVNALKDAGINTDQVHYLNAHGTSTPLGDKNESDAIKAAFGDQAYKMVVNSTKSMTGHLLGGAGGLESVFTVLALHNQVSPPTINLDNQDPECDLDYVANTAREMKIEVAVKNNFGFGGTNGTLVFRRA

SEQ ID NO: 36 - protein, 326 aa, Cupriavidus necator
MTKYAKIIGTGSYLPPRRVTNHDLATQLAEKGIETSDDWIVSRSGISARHWAEPDVTSSDLAVKAAEQAIEAAGIDRQSIDLIIVATSTPDFVFPSTACIVQEKLGITNHCPAFDLQAVCSGFVYALATADKFIRSGSHRNVLVIGTEVFSRILDFNDRTTCVLFGDGAGAVLLSASEEPGILSTAMHSDGRHVDILCVPGNVAGGNITGNPFLHMDGQAVFKLAVNVLDKVAREAMEAASVTPGQIDWLIPHQANIRIMQGTAKKLGLPAERMVATVHEHGNTSAASIPLALDVAVRDGRIRAGQTVLMEGVGGGFTWGAVLLRM

SEQ ID NO: 37 - protein, 252 aa, Cupriavidus necator
MRLANKVAIITGGGSGFGEGIAHTFAREGANVVVADLREEAAERVAAAIRDAGGRARAVRADVSREAGTEAMREAALATFGDVHIVVNNAGTTHRNKPILEVTEEEFDRVYAVNVKSIYWSARTFIPHFRQRGGGVFVNIASTAGIRPRPGLVWYNGSKGAVITASKAMAAELGKDNIRVNCVNPVIGATALLEEFMGMPDTPENRARFLATIPMGRMSTPQDVANACLYLASDEAAFITGTCIEVDGGRCV

SEQ ID NO: 38 - protein, 149 aa, Cupriavidus necator
MSAAEIDIRKILKLLPHRYPFLLVDRVLEFEAQKRIKTLKNVTINEPYFQGHFPEQPVMPGVMILEALAQSAGLLTFGADMERKEGALYYFVGIDGARFKQVVYPGDQLHMNVTVERYIRGIWKFKAFATVDDKVACEAELMCTVKQAE

SEQ ID NO: 39 - protein, 262 aa, Cupriavidus necator
MGFLAGKRILITGLLSNRSIAYGIASACKREGAELAFTYVGERFKDRISDFAKEFGSDLVFECDVGSDEQIAATFAALGQRWDKFDGLVHSIGFAPREAIAGDFLEGLSREGFRIAHDISAYSFPALAKAALPLLSDKASLLTLTYLGAERVVPNYNTMGLAKASLEASVRYLASSVGPRGIRANGISAGPIKTLAASGIKGFGKLLGHFEQAAPLRRNVTIEEVGNVAAFLLSDLASGVTGEITYVDGGFNVVGASVADAE

SEQ ID NO: 40 - protein, 300 aa, Umbellularia californica
MLEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRV

SEQ ID NO: 41 - protein, 661 aa, Marinobacter sp.
MNYFVTGGTGFIGRFLIAKLLARGAIVHVLVREQSVQKLADLREKLGADEKQIKAVVGDLTAPSLGLDKKTLKQLSGKIDHFFHLAAIYDMSASEESQQAANIDGTRAAVAAAEALEAGIFHHVSSIAVAGLFKGTFREDMFAEAGKLDHPYFRTKHESERVVRDDCKVPFRIYRPGLVIGDSATGDMDKVDGPYYFFKMIQKIRGALPQWVPTIGIEGGRLNIVPVNFVADALDHIAHLPNEDGKCFHLVDSDPYKVGEILNIFCEAGHAPKMGMRIDSRMFGFVPPFIRQSLKNLPPVKRMGRALLDDLGIPASVLSFINYPTRFDARETERVLQGTGIEVPRLPDYAPVIWDYWERNLDPDLFKDRTLRGTVEGKVCVVTGATSGIGLATAEKLADAGAILVIGARTQETLDQVSAQLNARGADVHAYQCDFADMDACDRFIQTVSENHGAVDVLINNAGRSIRRSLDKSFDRFHDFERTMQLNYFGSLRLIMGFAPAMLERRRGHIINISSIGVLTNSPRFSAYVASKSALDSFSRCAAAEWSDRRVCFTTINMPLVKTPMIAPTKIYDSVPTLSPEEAADMVVNAIVYRPKRIATRMGVFAQVLNAVAPKASEILMNTGYKMFPDSMPKKGKEVSAEKGASTDQVAFAAIMRGIHW

(19) United States
(12) Patent Application Publication
(10) Pub. No.: US 2026/0015572 A1
(43) Pub. Date: Jan. 15, 2026

(54) MICROORGANISMS AND METHODS FOR THE CONTINUOUS PRODUCTION OF FATTY ACID DERIVED PRODUCTS THROUGH THE EXPRESSION OF 3-HYDROXYACYL-ACP:COA TRANSACYLASES

(71) Applicant: LanzaTech, Inc., Skokie, IL (US)

(72) Inventor: James MacAllister Clomburg, Chicago, IL (US)

(21) Appl. No.: 19/174,388

(22) Filed: Apr. 9, 2025

Related U.S. Application Data
(60) Provisional application No. 63/670,068, filed on Jul. 11, 2024.

(51) Int. Cl. C12N 1/20; C12N 9/00; C12N 9/02; C12N 9/04; C12N 9/10; C12N 9/12; C12N 9/16; C12N 9/88; C12P 7/04; C12P 7/40; C12R 1/01

(52) U.S. Cl. CPC C12N 1/20; C12N 9/0006; C12N 9/0008; C12N 9/001; C12N 9/1029; C12N 9/1051; C12N 9/1217; C12N 9/16; C12N 9/88; C12N 9/93; C12P 7/04; C12P 7/40; C12R 2001/01; C12Y 101/011; C12Y 102/01042; C12Y 102/01084; C12Y 103/01044; C12Y 203/01018; C12Y 203/01019; C12Y 203/01039; C12Y 203/01041; C12Y 203/01179; C12Y 203/0118; C12Y 207/02007; C12Y 301/0202; C12Y 402/01119; C12Y 604/01002

(57) ABSTRACT

Microorganisms are genetically engineered to continuously produce fatty acids, fatty alcohols, cultured protein, or any combination thereof by microbial fermentation, particularly by microbial fermentation of a gaseous substrate. The microorganisms are C1-fixing. The production of fatty acids, fatty alcohols, and cultured proteins can be improved. This can be improved through the expression of 3-hydroxyacyl-ACP:CoA transacylases.

### CROSS REFERENCE TO RELATED APPLICATIONS

This application claims the benefit of U.S. Provisional Patent Application No. 63/670,068 filed on Jul. 11, 2024, the entirety of all of which is incorporated herein by reference.

### REFERENCE TO A SEQUENCE LISTING

The application contains a Sequence Listing which has been submitted electronically in ST.26 Sequence listing XML format and is hereby incorporated by reference in its entirety. Said ST.26 Sequence listing XML, created on Apr. 8, 2025, is named LT298US1-Sequences.xml and is 54,816 bytes in size.

### FIELD

The present disclosure relates to genetically engineered microorganisms and methods for the continuous production of fatty acids, fatty alcohols, cultured protein, or any combination thereof, particularly by microbial fermentation of a gaseous substrate.

### BACKGROUND

Mitigation of impending climate change requires drastic changes in manufacturing and greater reliance on biotechnology. Sustainable sources of fuels and chemicals are currently insufficient to significantly displace dependence on fossil carbon. Biotechnology harnesses the power of biology to create new products in a way that improves the quality of life and the environment, Gas fermentation is emerging as a powerful biotechnological advancement as an alternative platform for the biological fixation of such gases such as CH4, CO, CO2, and/or H2 into sustainable fuels and chemicals. In particular, gas fermentation technology can utilize a wide range of feedstocks including gasified carbon-containing matter such as municipal solid waste or agricultural waste, or industrial waste gases such as off-gases from steel manufacturing, petroleum refineries, and petrochemical processes to produce ethanol, aviation fuel, chemicals, and a variety of other products. Gas fermentation alone could displace 30% of crude oil use and reduce global CO2 emissions by 10%. As with any disruptive technology, many technical challenges must be overcome before this potential is fully achieved. The science of scale-up production and the reduction of obstacles for continued commercialization of gas fermentation are advanced by this disclosure.

Gas fermentation processes can be used to generate target materials from gas substrates or other input materials, particularly carbon-based materials. Greenhouse gases are an example of gas substrates. For example, particular biological systems can be used to perform gas fermentation. The production of longer carbon chain products (C6+) intrinsically requires the formation of carbon-carbon bonds to generate the required chain length intermediates needed to synthesize chemical products of interest (e.g., fatty alcohols, fatty acids, methyl ketones, etc.). While the engineering of fatty acid biosynthesis has been utilized to produce a range of products, in many cases generating the required pool of ACP intermediates and/or identifying enzymes to convert the ACP intermediates to a product of interest (e.g., acyl-ACP reductases) remains a significant challenge. An engineered reversal of the beta-oxidation cycle, utilizing CoA intermediates, represents an alternative pathway for producing fatty acid derived products that can utilize a wide range of product formation enzymes that operate on CoA intermediates. However, engineering high flux through this pathway inherently requires redirecting carbon away from biosynthetic pathways, such as fatty acid synthesis, and overcoming limitations in pathway thermodynamics. As an alternative to these individual pathways, the use of a 3-hydroxyacyl-ACP:CoA transacylase which converts an ACP intermediate involved in fatty acid biosynthesis to a CoA intermediate involved in beta-oxidation can capitalize on high flux, thermodynamically favorable fatty acid biosynthesis with wide range of enzymes for product synthesis that function on CoA intermediates. The ability to couple a high flux, thermodynamically favorable biosynthetic pathway with diverse CoA-dependent biochemical reactions is especially advantageous in chemolithoautotrophic organisms, in which C1 fixation is often a limiting step in the rates of growth and/or product synthesis. In this context, the use of 3-hydroxyacyl-ACP:CoA transacylases can enable product synthesis out of CoA intermediates, accessing the diverse biochemical reactions, without significantly altering flux in central carbon metabolism and to biosynthetic pathways toward high rates and productivities of product synthesis. There remains a need to reduce greenhouse gas emissions and provide alternate routes to sustainable chemicals production.

### SUMMARY

It is against the above background that the present disclosure provides certain advantages and advancements over the prior art.

Although this disclosure disclosed herein is not limited to specific advantages or functionalities, the disclosure provides for fatty acid and alcohol production in C1-fixing microorganisms with 3-hydroxyacyl-ACP:CoA transacylases.

One embodiment is directed to a method for the continuous production of at least one C₆₋₁₈ fatty acid and/or at least one C₆₋₁₈ fatty alcohol, the process comprising: passing a gaseous substrate to a bioreactor containing a culture of a recombinant C1-fixing microorganism according to claim 1, in a culture medium such that the microorganism converts the gaseous substrate to at least one C₆₋₁₈ fatty acid and/or at least one C₆₋₁₈ fatty alcohol; and recovering the C₆₋₁₈ fatty acid and/or C₆₋₁₈ fatty alcohol from the bioreactor.

The method according to an embodiment, wherein the microorganism further comprises one or more termination enzymes is selected from a) alcohol forming acyl-CoA reductase, aldehyde forming acyl-CoA reductase and aldehyde reductase having EC number 1.2.1.84, 1.2.1.B25, or 1.2.1.42; b) thioesterase having EC number 3.1.2.20; c) carboxylic acid reductase having EC number 1.2.1.- and aldehyde reductase having EC number 1.2.1.84, 1.2.1.B25, or 1.2.1.42; d) phosphate acyltransferase having EC number 2.3.1.18, 2.3.1.19, or 2.3.1.274 and carboxylic acid kinase having EC number 2.7.2.7, 2.7.2.18, 2.7.2.-; e) carboxylic acid reductase having EC number 1.2.1.-; f) aldehyde reductase having EC number 1.2.1.84, g) or any combination thereof.

The method of another embodiment, wherein the microorganism further comprises one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, and an acyl-CoA reductase.

A recombinant C1-fixing microorganism capable of producing at least one C₆₋₁₈ fatty acid, at least one C₆₋₁₈ fatty alcohol, or any combination thereof, from a gaseous substrate comprising a nucleic acid encoding a group of exogenous enzymes comprising a) acetyl-CoA carboxylase having EC number 6.4.1.2; b) [acyl-carrier-protein]S-malonyltransferase having EC number 2.3.1.39; c) 3-oxoacyl-[acyl-carrier-protein]synthase I, II, or III having EC number 2.3.1.41, 2.3.1.179, or 2.3.1.180; d) 3-oxoacyl-[acyl-carrier-protein]reductase having EC number 1.1.1.100; e) 3-hydroxyacyl-[acyl-carrier-protein]:CoA transacylase having EC number 2.4.1.-; f) (R)-specific enoyl-CoA hydratase having EC number 4.2.1.119; g) trans-2-enoyl-CoA reductase having EC number 1.3.1.44; and h) one or more termination enzymes.

The microorganism according to an embodiment, wherein the one or more termination enzymes is selected from a) thioesterase having EC number 3.1.2.20; b) phosphate acyltransferase having EC number 2.3.1.18, 2.3.1.19, or 2.3.1.274; c) carboxylic acid kinase having EC number 2.7.2.7, 2.7.2.18, or 2.7.2.-; or d) any combination thereof.

The microorganism according to an embodiment, wherein the one or more termination enzymes is selected from a) alcohol forming acyl-CoA reductase, aldehyde forming acyl-CoA reductase and aldehyde reductase having EC number 1.2.1.84, 1.2.1.B25, or 1.2.1.42; b) thioesterase having EC number 3.1.2.20; c) carboxylic acid reductase having EC number 1.2.1.- and aldehyde reductase having EC number 1.2.1.84, 1.2.1.B25, or 1.2.1.42; d) phosphate acyltransferase having EC number 2.3.1.18, 2.3.1.19, or 2.3.1.274 and carboxylic acid kinase having EC number 2.7.2.7, 2.7.2.18, 2.7.2.-; e) carboxylic acid reductase having EC number 1.2.1.-; f) aldehyde reductase having EC number 1.2.1.84, g) or any combination thereof.

A process for continuous co-production of at least one C₆₋₁₈ fatty acid or C₆₋₁₈ fatty alcohol and cultured protein comprising: a) providing a continuous bioreactor; b) introducing to the bioreactor a recombinant C1-fixing microorganism according to claim 1, a gaseous substrate comprising one or more of CO, CO₂, and H₂, and a liquid growth medium; c) continuously culturing the recombinant C1-fixing microorganism thereby generating a gas fermentation broth comprising 1) the at least one C₆₋₁₈ fatty acid or C₆₋₁₈ fatty alcohol, and 2) cultured protein; d) continuously removing a portion of the gas fermentation broth in a first stream; e) continuously removing the at least one C₆₋₁₈ fatty acid or C₆₋₁₈ fatty alcohol in a second stream; and continuously recovering the cultured protein from the first stream.

The microorganism according to another embodiment, wherein the microorganism is selected from the group consisting of Cupriavidus necator and Ralstonia eutropha.

The microorganism according to an embodiment, wherein the microorganism is Cupriavidus necator.

The method according to an embodiment, wherein the microorganism is selected from DSM 428, DSM 531, DSM 541, or DSM 34774.

The microorganism according to an embodiment, wherein the 3-hydroxyacyl-[acyl-carrier-protein]:CoA transacylase is (R)-3-hydroxydecanoyl-ACP:CoA transacylase (PhaG).

In some aspects of the microorganism disclosed herein, the microorganism is directed to a recombinant C1-fixing microorganism capable of producing C₆₋₁₈ fatty acids, C₆₋₁₈ fatty alcohols, or any combination thereof, from a gaseous substrate comprising a nucleic acid encoding a group of exogenous enzymes comprising thiolase; 3-hydroxyacyl-CoA dehydrogenase or 3-oxoacyl-CoA reductase; 3-hydroxyacyl-CoA dehydratase or enoyl-CoA hydratase; enoyl-CoA reductase; and one or more termination enzymes.

The microorganism of an embodiment, wherein the one or more termination enzymes is selected from thioesterase, phosphate acyltransferase, carboxylic acid kinase, or any combination thereof.

The microorganism of an embodiment, wherein the one or more termination enzymes is selected from alcohol forming acyl-CoA reductase, aldehyde forming acyl-CoA reductase and aldehyde reductase, thioesterase, carboxylic acid reductase and aldehyde reductase, phosphate acyltransferase and carboxylic acid kinase, carboxylic acid reductase, aldehyde reductase, or any combination thereof.

Another aspect of the microorganism is directed to A recombinant C1-fixing microorganism capable of producing C₆₋₁₈ fatty acids, C₆₋₁₈ fatty alcohols, or any combination thereof, from a gaseous substrate comprising a nucleic acid encoding a group of exogenous enzymes comprising: acetyl-CoA carboxylase; [acyl-carrier-protein]S-malonyltransferase; 3-oxoacyl-[acyl-carrier-protein]synthase I, II, and/or III; 3-oxoacyl-[acyl-carrier-protein]reductase; 3-hydroxyacyl-[acyl-carrier-protein]dehydratase; enoyl-[acyl-carrier-protein]reductase; and one or more termination enzymes.

The microorganism of an embodiment, wherein the one or more termination enzymes is acyl-ACP-thioesterase.

The microorganism of an embodiment, wherein the one or more termination enzymes is selected from alcohol forming acyl-ACP reductase, aldehyde forming acyl-ACP reductase, aldehyde reductase, acyl-ACP thioesterase, carboxylic acid reductase, aldehyde reductase, phosphate acyltransferase, carboxylic acid kinase, carboxylic acid reductase, aldehyde reductase, or any combination thereof.

The microorganism of an embodiment, wherein the microorganism is selected from the group consisting of Cupriavidus necator and Ralstonia eutropha.

The microorganism of an embodiment, wherein the microorganism is Cupriavidus necator.

The microorganism of an embodiment, further comprising an acyl-CoA primer and extender, wherein the primer and extender are capable of cyclic, iterative pathway operation.

The microorganism of an embodiment, further comprising one or more inducible promoters.

The microorganism of an embodiment, wherein the one or more inducible promoters is selected from an H₂ inducible promoter, a phosphate limited inducible promoter, a nitrogen limited inducible promoter, a CO₂ inducible promoter, or any combination thereof.

The microorganism of an embodiment, further comprising a disruptive mutation in one or more genes.

The microorganism of an embodiment, wherein the gaseous substrate comprises CO₂ and an energy source.

One embodiment is directed to a method for the continuous production of C₆₋₁₈ fatty acids and/or C₆₋₁₈ fatty alcohols, the process comprising: passing a gaseous substrate to a bioreactor containing a culture of a recombinant C1-fixing microorganism according to claim 1, in a culture medium such that the microorganism converts the gaseous substrate to C₆₋₁₈ fatty acids and/or C₆₋₁₈ fatty alcohols; and recovering the C₆₋₁₈ fatty acids and/or C₆₋₁₈ fatty alcohols from the bioreactor.

The microorganism of an embodiment, wherein the gaseous substrate comprises an industrial waste product or off-gas.

The microorganism of an embodiment, further comprising an energy source.

The microorganism of an embodiment, wherein the energy source is provided intermittently.

The microorganism of an embodiment, wherein the gaseous substrate comprises CO₂ and an energy source.

The microorganism of an embodiment, wherein the energy source is H₂.

The microorganism of an embodiment, wherein the gaseous substrate further comprises H₂, O₂, or both.

The microorganism of an embodiment, further comprising a step of limiting dissolved oxygen concentration, thereby switching a cellular burden.

Another embodiment is directed to a process for continuous co-production of at least one C₆₋₁₈ fatty acid or C₆₋₁₈ fatty alcohol and cultured protein comprising:
a) providing a continuous bioreactor;
b) introducing to the bioreactor a recombinant C1-fixing microorganism capable of co-producing at least one C₆₋₁₈ fatty acid or C₆₋₁₈ fatty alcohol, a gaseous substrate comprising one or more of CO, CO₂, and H₂, and a liquid growth medium;
c) continuously culturing the recombinant C1-fixing microorganism thereby generating a gas fermentation broth comprising 1) the at least one C₆₋₁₈ fatty acid or C₆₋₁₈ fatty alcohol, and 2) cultured protein;
d) continuously removing a portion of the gas fermentation broth in a first stream;
e) continuously removing the at least one C₆₋₁₈ fatty acid or C₆₋₁₈ fatty alcohol in a second stream; and continuously recovering the cultured protein from the first stream.

One embodiment is directed to a method for continuous co-production of at least one C₆₋₁₈ fatty acid or C₆₋₁₈ fatty alcohol and cultured protein, the method comprising: a) culturing, in a bioreactor, a recombinant C1-fixing microorganism capable of producing at least one C₆₋₁₈ fatty acid or C₆₋₁₈ fatty alcohol in the presence of a gaseous substrate comprising one or more of CO, CO₂, and H₂, and a liquid growth medium; b) generating cultured protein having a unit value, at least one C₆₋₁₈ fatty acid or C₆₋₁₈ fatty alcohol having a unit value, wherein the unit value of the cultured protein is greater than the unit value of the C₆₋₁₈ fatty acid or C₆₋₁₈ fatty alcohol; and c) recovering the cultured protein in an amount of at least 15% of a sum value of the unit value of the cultured protein and the unit value of the C₆₋₁₈ fatty acid or C₆₋₁₈ fatty alcohol.

The microorganism of an embodiment, wherein recovering of step c) of the cultured protein is in an amount of at least 1% of the sum value.

The microorganism of an embodiment, wherein the C₆₋₁₈ fatty acid is selected from hexanoic acid, octanoic acid, decanoic acid, dodecanoic acid, tetradecanoic acid, hexadecanoic acid, octadecanoic acid, palmitoleic acid, vaccenic acid, octadec-9-enoic acid, 8-(2-hexylcyclopropyl)octanoic acid, 8-(2-octylcyclopropyl)octanoic acid, 10-(2-hexylcyclopropyl)decanoic acid, 3-hydroxy-Cₙ-acid (i.e., 3-hydroxy-tetradecanoic acid), C₁-2-enoic acid (i.e., tetradec-2-enoic acid), or any combination thereof.

The microorganism of an embodiment, wherein the C₆₋₁₈ fatty acid is a branched-chain acid selected from anteiso-C13:0 (anteisotridecanoic acid), iso-C13:0 (isotridecanoic acid), anteiso-C14:0 (anteisotetradecanoic acid), iso-C14:0 (isotetradecanoic acid), anteiso-C15:0 (anteisopentadecanoic acid), iso-C15:0 (isopentadecanoic acid), anteiso-C16:0 (anteisohexadecanoic acid), iso-C16:0 (isohexadecanoic acid), anteiso-C17:0 (anteisoheptadecanoic acid), iso-C17:0 (isoheptadecanoic acid), anteiso-C18:0 (anteisooctadecanoic acid), iso-C18:0 (isooctadecanoic acid), anteiso-C19:0 (anteisononadecanoic acid), iso-C19:0 (isononadecanoic acid), or any combination thereof.

The microorganism of an embodiment, wherein the C₆₋₁₈ fatty alcohol is selected from hexanol, octanol, decanol, dodecanol, tetradecanol, hexadecanol, octadecanol, hexadec-9-en-1-ol, octadec-11-en-1-ol, Octadec-9-en-1-ol, 3-hydroxy-Cₙ-1-ol, Cₙ-2-en-1-ol, or any combination thereof.

The microorganism of an embodiment, wherein the C₆₋₁₈ fatty alcohol is a branched-chain alcohol selected from anteiso-C13:0 (anteisotridecanol), iso-C13:0 (isotridecanol), anteiso-C14:0 (anteisotetradecanol), iso-C14:0 (isotetradecanol), anteiso-C15:0 (anteisopentadecanol), iso-C15:0 (isopentadecanol), anteiso-C16:0 (anteisohexadecanol), iso-C16:0 (isohexadecanol), anteiso-C17:0 (anteisoheptadecanol), iso-C17:0 (isoheptadecanol), anteiso-C18:0 (anteisooctadecanol), iso-C18:0 (isooctadecanol), or any combination thereof.

The microorganism of an embodiment, further comprising the cultured protein and at least one excipient.

The microorganism of an embodiment, wherein the cultured protein is suitable as animal feed.

The microorganism of an embodiment, wherein the cultured protein is suitable as food.

The disclosure is directed to a genetically engineered microorganism and methods for the production of primary alcohols, 1,4-diols, 1,6-diols, diacids, trans Δ² fatty alcohols, β-keto alcohols, 1,3-diols, β-hydroxy acids, carboxylic acids, or hydrocarbons by microbial fermentation of a substrate comprising CO, CO₂, and/or H₂, and recombinant microorganisms of use in such methods.

In a first aspect, the disclosure provides an genetically recombinant microorganism capable of producing from primary alcohols, 1,4-diols, 1,6-diols, diacids, trans Δ² fatty alcohols, 3-keto alcohols, 1,3-diols, β-hydroxy acids, carboxylic acids, or hydrocarbons and optionally one or more other products by fermentation of a substrate comprising CO, CO₂, and/or H₂.

In one particular embodiment, the microorganism is adapted to express one or more enzymes (or one or more subunits thereof) in the reverse β-oxidation pathway, e.g., in a reverse biosynthetic direction, which enzymes are not naturally present in a parental microorganism from which the recombinant microorganism is derived. In another embodiment, the microorganism is adapted to over-express one or more enzymes (or one or more subunits thereof) in the reverse β-oxidation pathway, which enzymes are naturally present in a parental microorganism from which the recombinant microorganism is derived. In one embodiment, the microorganism is adapted to express one or more enzymes (or one or more subunits thereof) in the reverse β-oxidation pathway which are not naturally present in a parental microorganism and over-express one or more enzymes (or one or more subunits thereof) in the reverse β-oxidation pathway which are naturally present in a parental microorganism. The reverse β-oxidation pathway is also cyclic and iterative. In another embodiment, the β-oxidation pathway is driven in reverse for as many cycles, as desired. In one embodiment, the β-oxidation pathway is expressed in the absence of any naturally occurring substrates. In one embodiment, the reverse β-oxidation pathway is functionally expressed. In one embodiment, the CoA thioester intermediates can be converted to useful products by the action of different types of termination enzymes.

This pathway operates with coenzyme-A (CoA) thioester intermediates and directly uses, for example, acetyl-CoA for acyl-chain elongation, and characteristics that enable product synthesis used in combination with endogenous dehydrogenases and thioesterases to synthesize (Cₙ)-alcohols, fatty acids, β-hydroxy-, β-keto-, and trans-Δ²-carboxylic acids.

This pathway can be further extended using the same enzymes or engineered variants thereof that have specificity for higher chain length to produce, including but not limited to, a range of C4, C6, C8, C10, C12, C14 alcohols, ketones, enols or diols. Different type of molecules can be obtained also by using primer or extender units different than acetyl-CoA in a thiolase step.

In one embodiment, a genetically engineered microorganism capable of producing a product from a gaseous substrate, wherein the microorganism comprises an iterative pathway comprising:
a nucleic acid encoding a group of enzymes that are capable of catalyzing the conversion of (Cₙ)-acyl CoA to β-ketoacyl-CoA;
a nucleic acid encoding a group of exogenous enzymes capable of catalyzing the conversion of β-ketoacyl-CoA to β-hydroxyacyl-CoA;
a nucleic acid encoding a group of exogenous enzymes capable of catalyzing the conversion of β-hydroxyacyl-CoA to trans-Δ²-Enoyl-CoA;
a nucleic acid encoding a group of exogenous enzymes capable of catalyzing the conversion of trans-Δ²-Enoyl-CoA to (Cₙ₊₂) acyl-CoA;
one or more termination enzymes; and wherein the microorganism is a C1-fixing bacteria comprising a disruptive mutation in a thioesterase.

In one embodiment, the nucleic acid encoding a group of enzymes that are capable of catalyzing the conversion of (Cₙ)-acyl CoA to β-ketoacyl-CoA is a thiolase, an acyl-CoA acetyltransferase, or a polyketide synthase; the nucleic acid encoding a group of enzymes that are capable of catalyzing the conversion of β-ketoacyl-CoA to β-hydroxyacyl-CoA is a β-Ketoacyl-CoA reductase or a β-hydroxyacyl-CoA dehydrogenase; the nucleic acid encoding a group of exogenous enzymes capable of catalyzing the conversion of β-ketoacyl-CoA to trans-Δ²-Enoyl-CoA is a β-hydroxyacyl-CoA dehydratase; the nucleic acid encoding a group of exogenous enzymes capable of catalyzing the conversion of trans-Δ²-Enoyl-CoA to (Cₙ₊₂) acyl-CoA is a trans-Enoyl-CoA reductase or butyryl-CoA dehydrogenase/electron transferring flavoprotein AB (Bcd-EtfAB).

In one embodiment, a nucleic acid encoding a group of termination enzymes is selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, an acyl-CoA reductase, or any combination thereof. In one embodiment, a termination enzyme is phosphate butyryltransferase (Ptb) and exogenous butyrate kinase (Buk) (Ptb-Buk). In one embodiment, a termination enzyme is thioesterase. In one embodiment, one or more termination enzymes are selected.

In one embodiment, the microorganism comprises one or more exogenous nucleic acids adapted to increase expression of one or more nucleic acids native to the parental microorganism and which one or more nucleic acids encode one or more of the enzymes (or one or more subunits thereof) referred to herein before.

In one embodiment, the one or more exogenous nucleic acid adapted to increase expression is a regulatory element. In one embodiment, the regulatory element is a promoter.

In one embodiment, the promoter is a constitutive promoter. In one embodiment, the promoter is selected from the group comprising Phosphotransacetylase/Acetate kinase operon promoters.

In one embodiment, the microorganism comprises one or more exogenous nucleic acids encoding and adapted to express one or more of the enzymes (or one or more subunits thereof) referred to herein before. In one embodiment, the microorganisms comprise one or more exogenous nucleic acid encoding and adapted to express at least two of the enzymes (or one or more subunits thereof).

In one embodiment, the one or more exogenous nucleic acid is a nucleic acid construct or vector, in one particular embodiment a plasmid, encoding one or more of the enzymes referred to hereinbefore in any combination.

In one embodiment, two or more enzymes on a plasmid are arranged in a single operon in any order, or in multiple operons in any order.

In one embodiment, the exogenous nucleic acid is an expression plasmid.

In one embodiment, the parental microorganism is selected from the group of anaerobic acetogens.

In a second aspect, the disclosure provides a nucleic acid encoding one or more enzymes (or one or more subunits thereof) which when expressed in a microorganism allows the microorganism to produce Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids, or any combination thereof, by fermentation of substrate comprising CO and/or CO₂.

For example, a Cₙ₊₂ ketone may be acetone.

In one embodiment, the nucleic acid encodes two or more enzymes (or one or more subunits thereof) which when expressed in a microorganism allows the microorganism to produce primary alcohols, 1,4-diols, 1,6-diols, diacids, trans Δ² fatty alcohols, β-keto alcohols, 1,3-diols, β-hydroxy acids, carboxylic acids, or hydrocarbons, by fermentation of substrate comprising CO.

In one embodiment, the enzymes are chosen from thiolase, acyl-CoA acetyltransferase, or a polyketide synthase, and/or a functionally equivalent variant of any one or more thereof.

In one embodiment, the nucleic acid comprises nucleic acid sequences encoding β-Ketoacyl-CoA reductase and/or a β-hydroxyacyl-CoA dehydrogenase, or a functionally equivalent variant of any one or more thereof, in any order.

In one embodiment, the nucleic acids of the disclosure further comprise a promoter. In one embodiment, the promoter allows for constitutive expression of the genes under its control. In another particular embodiment, a Phosphotransacetylase/Acetate kinase operon promoter is used.

In a third aspect, the disclosure provides a nucleic acid construct or vector comprising one or more nucleic acid of the second aspect.

In one particular embodiment, the nucleic acid construct or vector is an expression construct or vector. In one particular embodiment, the expression construct or vector is a plasmid.

In a fourth aspect, the disclosure provides host organisms comprising any one or more of the nucleic acids of the seventh aspect or vectors or constructs of the third aspect.

In a fifth aspect, the disclosure provides a composition comprising an expression construct or vector as referred to in the third aspect of the disclosure and a methylation construct or vector.

Preferably, the composition is able to produce a recombinant microorganism according to the first aspect of the disclosure.

In one particular embodiment, the expression construct/vector and/or the methylation construct/vector is a plasmid.

In a sixth aspect, the disclosure provides a method of producing a product, the method comprising culturing the engineered microorganism of claim 1, in the presence of a gaseous substrate.

In one embodiment the method comprises wherein the gaseous substrate comprises a C1-carbon source comprising CO, CO₂, and/or H₂.

In one embodiment the method comprises wherein the product is selected from (Cₙ)-alcohols, primary alcohols, trans Δ² fatty alcohols, j-keto alcohols, 1,3-diols, 1,4-diols, 1,6-diols, diacids, β-hydroxy acids, β-ketoacids carboxylic acids, fatty acids, fatty acid methyl esters, ketoacids, hydrocarbons, or any combination thereof.

In particular embodiments of the method aspects, the microorganism is maintained in an aqueous culture medium.

In particular embodiments of the method aspects, the fermentation of the substrate takes place in a bioreactor.

Preferably, the substrate comprising CO and/or CO₂ is a gaseous substrate comprising CO and/or CO₂. In one embodiment, the substrate comprises an industrial waste gas. In certain embodiments, the gas is steel mill waste gas or syngas.

In a particular embodiment, the substrate is a substrate comprising CO.

In embodiments of the disclosure where the substrate comprises CO₂, but no CO, the substrate preferably also comprises H₂.

In one embodiment, the substrate comprises CO and CO₂. In one embodiment, the substrate comprises CO₂ and H₂. In another embodiment, the substrate comprises CO, CO₂, and H₂.

In one embodiment, the substrate will typically contain a major proportion of CO, such as at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

In certain embodiments the methods further comprise the step of recovering a product selected from primary alcohols, 1,4-diols, 1,6-diols, diacids, trans Δ² fatty alcohols, β-keto alcohols, 1,3-diols, β-hydroxy acids, carboxylic acids, or hydrocarbons, and optionally one or more other products from the fermentation broth.

In a seventh aspect, the disclosure provides any primary alcohol when produced by the method of the sixth aspect.

In another aspect, the disclosure provides a method for the production of a microorganism of the first aspect of the disclosure comprising transforming a parental microorganism with one or more exogenous nucleic acid such that the microorganism is capable of producing Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids. or any combination thereof, and optionally one or more other products, by fermentation of a substrate comprising CO and/or CO₂, wherein the parental microorganism is not capable of producing Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids. or any combination thereof by fermentation of a substrate comprising CO and/or CO₂.

In one particular embodiment, a parental microorganism is transformed with one or more exogenous nucleic acid adapted to express one or more enzymes in the reverse 3-oxidation pathway which are not naturally present in the parental microorganism. In another embodiment, a parental microorganism is transformed with one or more nucleic acid adapted to over-express one or more enzymes in the reverse β-oxidation pathway which are naturally present in the parental microorganism. In another embodiment, a parental microorganism is transformed with one or more exogenous nucleic acid adapted to express one or more enzymes in the reverse β-oxidation pathway which are not naturally present in the parental microorganism and over-express one or more enzymes in the reverse β-oxidation pathway which are naturally present in the parental microorganism.

In certain embodiments, the one or more enzymes are as herein before described.

In certain embodiment, the parental microorganism is as herein before described.

According to one embodiment a process is provided for converting CO or CO2 into primary alcohol. A gaseous CO-containing and/or CO2-containing substrate is passed to a bioreactor containing a culture of C1-fixing bacteria in a culture medium such that the bacteria convert the CO and/or CO2 to a primary alcohol. The C1-fixing bacteria are genetically engineered to express an enzyme in the reverse β-oxidation pathway. They also express an enzyme in the reverse β-oxidation pathway, whether native or exogenous. The primary alcohol is recovered from the bioreactor.

According to another embodiment an isolated, genetically engineered, C1-fixing bacterium is provided that comprises a nucleic acid encoding a group of enzymes in the reverse β-oxidation pathway. The nucleic acid is exogenous to the host bacteria. The bacteria express the group of enzymes in the reverse β-oxidation pathway and the bacteria acquire the ability to generate primary alcohols, trans Δ² fatty alcohols, β-keto alcohols, 1,3-diols, 1,4-diols, 1,6-diols, diacids, β-hydroxy acids, carboxylic acids, or hydrocarbons. The group of enzymes in the pathway are typically at least 85% identical to the amino acid sequence encoded by any one of the nucleotide sequences of SEQ ID NO: 14 to SEQ ID NO: 41. In one embodiment, termination enzymes such as thioesterases, acyl-CoA reductases, or phosphotransacylase, carboxylate kinase, are selected because these pull the acyl-CoA intermediates from the rBOX pathway and drive the metabolic flux through the pathway.

The bacteria may further comprise an exogenous nucleic acid encoding acetyl-Coenzyme A carboxylase. The nucleic acid may be operably linked to a promoter. The nucleic acid may have been codon optimized. The nucleic acid or the encoded carboxylase may be from a nonsulfur, photosynthetic bacterium.

The genetically engineered bacteria may be cultured by growing in a medium comprising a gaseous carbon source. The carbon source may comprise CO and/or CO₂, which may be used as either or both of an energy source or a carbon source. The bacteria may optionally be grown under strictly anaerobic conditions. The carbon source may comprise an industrial waste product or off-gas.

In some aspects of the method disclosed herein, the method is directed to a process for continuous co-production of at least one chemical product and at least one exogenous gene product comprising:
a) providing a continuous bioreactor;
b) introducing to the bioreactor a recombinant C1-fixing microorganism capable of co-producing at least one chemical product and at least one exogenous gene product, a gaseous substrate comprising one or more of CO, CO₂, and H₂, and a liquid growth medium;
c) continuously culturing the recombinant C1-fixing microorganism thereby generating a gas fermentation broth comprising 1) the at least one chemical product, 2) the at least one exogenous gene product, and 3) microbial biomass;
d) continuously removing a portion of the gas fermentation broth in a first stream;
e) continuously removing the at least one chemical product in a second stream; and
f) continuously recovering the at least one exogenous gene product from the microbial biomass from the first stream.

In some aspects of the method disclosed herein, the method is directed to a method for the continuous co-production of at least one targeted chemical product and at least one exogenous gene product, the method comprising: a) culturing a recombinant C1-fixing microorganism capable of co-production of at least one targeted chemical product and at least one exogenous gene product in the presence of a gaseous substrate comprising one or more of CO, CO₂, and H₂, wherein the culturing is a continuous fermentation process; and wherein the substrate and liquid nutrient medium of the culture are non-coalescing.

Another aspect is directed to a method for continuous co-production of at least one targeted chemical product and at least one exogenous gene product, the method comprising: a) culturing in a state of a continuous gas fermentation process, a recombinant C1-fixing microorganism capable of co-production of at least one targeted chemical product and at least one exogenous gene product in a fermentation broth comprising the microorganism, a gaseous substrate comprising one or more of CO, CO₂, and H₂, and a liquid growth medium, wherein the fermentation broth comprises an equilibrium surface tension of from about 30 to about 40 mN/m.

One aspect is directed to a method for continuous co-production of at least one targeted chemical product and at least one exogenous gene product, the method comprising: a) culturing in a bioreactor, a recombinant C1-fixing microorganism capable of co-production of at least one targeted chemical product and at least one exogenous gene product having a unit value in the presence of a gaseous substrate comprising one or more of CO, CO₂, and H₂, and a liquid growth medium; and recovering the at least one targeted chemical product and the at least one exogenous gene product wherein the at least one exogenous gene product is recovered in an amount from about 0.1% to about 1% grams/dry cell weight/day of the at least one exogenous gene product produced.

The method of an embodiment, further comprising an initial stage of gas fermentation wherein the initial surface tension of the broth is from about 60 to about 72 mN/m.

The method of an embodiment, wherein the exogenous gene product has a high market value.

The method of an embodiment, wherein the exogenous gene product is a high-value, specialized protein.

The method of an embodiment, wherein the exogenous gene product is an antioxidant or an antioxidant enzyme.

One embodiment is directed to a method for continuous co-production of at least one targeted chemical product and at least one exogenous gene product, the method comprising: a) culturing, in a bioreactor, a recombinant C1-fixing microorganism capable of co-production of at least one targeted chemical product and at least one exogenous gene product in the presence of a gaseous substrate comprising one or more of CO, CO₂, and H₂, and a liquid growth medium; b) generating microbial biomass having a unit value, at least one targeted chemical product, and at least one exogenous gene product having a unit value, wherein the unit value of the exogenous gene product is greater than the unit value of the microbial biomass; and c) recovering the at least one exogenous gene product in an amount of at least 15% of a sum value of the unit value of the exogenous gene product and the unit value of the microbial biomass.

The method of an embodiment, wherein recovering of step c) of the at least one exogenous gene product is in an amount of at least 1% of the sum value.

The method of an embodiment, wherein the high-value, specialized protein is selected from ubiquinone, coenzyme Q10, copper/zinc and manganese-dependent superoxide dismutase, iron-dependent catalase, selenium-dependent glutathione peroxidase, albumin, ceruloplasmin, metallothionein, ferritin, myoglobin, transferrin, haptoglobins, ceruloplasmin, heat shock proteins, iron-dependent superoxide dismutase, nickel-dependent superoxide dismutase, or any combination thereof.

The method of an embodiment, wherein the catalases are selected from heme-containing catalases and non-heme manganese catalases.

The method of an embodiment, wherein the at least one chemical product is selected from 1-butanol, butyrate, butene, butadiene, methyl ethyl ketone, ethylene, acetone, isopropanol, lipids, 3-hydroxypropionate, terpenes, isoprene, fatty acids, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, monoethylene glycol, or any combination thereof.

The method of an embodiment, further comprising the recombinant microorganism comprising a disruptive mutation in one or more genes.

The method of an embodiment, further comprising a microbial biomass and at least one excipient.

The method of an embodiment, wherein the microbial biomass is suitable as animal feed.

In some aspects of the microorganism disclosed herein, the microorganism produces a commodity chemical product, a tandem repeat protein product, microbial biomass, cultured protein or single cell protein (SCP), one or more intermediates, or any combination thereof.

In some aspects of the microorganism disclosed herein, the microorganism produces a exogenous gene product. In one embodiment, the exogenous gene product comprises a exogenous nucleic acid encoding at least one protein having tandem repeats.

In some aspects of the microorganism disclosed herein, the microorganism comprises a genetically engineered microorganism capable of co-producing at least one heterologous protein and at least one secreted chemical product from a gaseous substrate, the microorganism comprising a heterologous nucleic acid encoding the at least one protein having tandem repeats and a heterologous nucleic acid encoding the at least one secreted chemical product, wherein the microorganism is a C1-fixing bacteria.

In some aspects of the microorganism disclosed herein, the microorganism comprises a genetically engineered C1-fixing microorganism capable of co-producing an heterologous protein and a chemical product from a gaseous substrate, the microorganism comprising a heterologous nucleic acid encoding the at least one heterologous protein having one or more tandem repeats and a heterologous nucleic acid encoding the at least one chemical product, wherein the microorganism is capable of accumulating the at least one heterologous protein in the cell and secreting the at least one chemical product from the cell.

In some aspects of the microorganism disclosed herein, the microorganism comprises a genetically engineered C1-fixing microorganism capable of co-producing at least one heterologous functional protein and at least one chemical product having two or more carbons from a gaseous substrate, the microorganism comprising a heterologous nucleic acid encoding at least one protein having tandem repeats and a heterologous nucleic acid encoding at least one secreted chemical product.

In some aspects of the microorganism disclosed herein, the microorganism comprises a genetically engineered C1-fixing microorganism capable of co-producing at least one heterologous functional protein and at least one chemical product having two or more carbons from a gaseous substrate, the microorganism comprising a heterologous nucleic acid encoding a group of genes comprising at least one protein having tandem repeats and at least one secreted chemical product.

In some aspects of the microorganism disclosed herein, the microorganism comprises a genetically engineered C1-fixing microorganism capable of co-producing at least one heterologous protein and at least one chemical product from a gaseous substrate, the microorganism comprising:
a) a heterologous nucleic acid encoding at least one heterologous protein having one or more tandem repeats; and
b) a heterologous nucleic acid encoding at least one chemical having two or more carbons, wherein the microorganism is capable of accumulating the at least one heterologous protein in the cell and secreting the at least one chemical product from the cell.

One aspect comprises a method of co-producing at least one heterologous protein and at least one chemical product by culturing the genetically engineered C1-fixing. microorganism in the presence of a gaseous substrate comprising one or more of CO, CO₂, and H₂, wherein the culturing is a continuous fermentation process.

One aspect comprises a method, wherein the gaseous substrate comprises a C1-carbon source comprising one or more of CO, CO₂, and H₂.

One aspect comprises a method, wherein the gaseous substrate comprises syngas or industrial waste gas.

In some aspects of the microorganism disclosed herein, the microorganism is derived from a parental bacterium selected from the group consisting of Cupriavidus necator.

In some aspects of the microorganism disclosed herein, where the at least one secreted chemical product is selected from the group 1-butanol, butyrate, butene, butadiene, methyl ethyl ketone, ethylene, acetone, isopropanol, lipids, 3-hydroxypropionate, terpenes, isoprene, fatty acids, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-01, isovalerate, isoamyl alcohol, or monoethylene glycol.

In some aspects of the microorganism disclosed herein, the microorganism further comprising a disruptive mutation in one or more genes.

The disclosure further provides the genetically engineered C1-fixing microorganism, further comprising a microbial biomass and at least one excipient.

The disclosure further provides the genetically engineered C1-fixing microorganism, wherein the microbial biomass is suitable as animal feed.

The disclosure further provides the genetically engineered C1-fixing microorganism, wherein the animal feed is suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The disclosure further provides the genetically engineered C1-fixing microorganism, wherein the microorganism is suitable as a cultured protein or single cell protein (SCP).

The disclosure further provides the genetically engineered C1-fixing microorganism, wherein the microorganism is suitable as a cell-free protein synthesis (CFPS) platform.

The disclosure further provides the genetically engineered C1-fixing microorganism, wherein the at least one secreted chemical product is native to the microorganism.

In some aspects of the method disclosed herein, the substrate comprises one or more of CO, CO₂, and H₂.

In some aspects of the method disclosed herein, at least a portion of the substrate is industrial waste gas, industrial off gas, or syngas.

In some embodiments, both anaerobic and aerobic gases can be used to feed separate cultures (e.g., an anaerobic culture and an aerobic culture) in two or more different bioreactors that are both integrated into the same process stream.

In some embodiments, the disclosure provides a method for storing energy in the form of a biopolymer comprising intermittently processing at least a portion of electric energy generated from a renewable and/or non-renewable energy source in an electrolysis process to produce at least H₂, O₂ or CO; intermittently passing at least one of H₂, O₂, or CO from the electrolysis process to a bioreactor containing a culture comprising a liquid nutrient medium and a microorganism capable of producing a biopolymer; and fermenting the culture.

In an embodiment, the disclosure also provides a system for storing energy in the form of biopolymer comprising an electrolysis process in intermittent fluid communication with a renewable and/or non-renewable energy source for producing at least one of H₂, O₂, or CO; an industrial plant for producing at least C1 feedstock; a bioreactor, in intermittent fluid communication with the electrolysis process and/or in continuous fluid communication with the industrial plant, comprising a reaction vessel suitable for intermittently growing, fermenting, and/or culturing and housing a microorganism capable of producing a biopolymer.

In some embodiments, the disclosure provides a method for improving the performance and/or the economics of a fermentation process, the fermentation process defining a bioreactor containing a bacterial culture in a liquid nutrient medium, wherein the method comprises passing a C1 feedstock comprising one or both of CO and CO₂ from an industrial process to the bioreactor, wherein the C1 feedstock has a cost per unit, intermittently passing at least one of H₂, O₂, or CO from the electrolysis process to the bioreactor, wherein the electrolysis process has a cost per unit, and fermenting the culture to produce one or more fermentation products, wherein each of the one or more fermentation products has a value per unit. In certain instances, multiple electrolysis processes are utilized in order to provide one or all of CO, CO₂, and H₂ to the bioreactor.

In another embodiment, the local power grid provides electricity intermittently passed as electrical energy produced by power based on availability of electrical power or the availability of electricity below a threshold price, where power prices fall as demand falls, or as set by the local power grid.

In an embodiment, the disclosure can be operated intermittently by storing energy in the form of a biopolymer, where product conversion can be intermittent during periods when an electricity grid is oversupplied with electricity, or idle when electricity is scarce or power is in demand. The disclosure provides a process that is capable of being fine-tuned to assist with balancing an electrical power grid system by storing energy in the form of a biopolymer.

In one embodiment an autotrophic microorganism intermittently consumes, in part or entirely, the energy provided by the availability of power.

In one embodiment, the systems disclosed herein relate to generating fine bubbles and may include a vessel containing a liquid, a plate comprising a plurality of orifices positioned in an upper portion of the vessel and configured to accelerate at least a portion of the liquid in the vessel, and at least one sparger positioned within the vessel with a surface of the sparger positioned from about 50 mm to about 300 mm, 500 mm, or 1000 mm from a bottom of the plate. The sparger may be configured to inject bubbles into the liquid. In some examples, the sparger may be positioned within the vessel to create a first zone for the bubbles to rise within the vessel, and to create a second zone for the accelerated liquid to break the bubbles into fine bubbles and for fluid to flow through the vessel. The fluid may include the accelerated portion of the liquid and fine bubbles. In still other examples, the superficial velocity of the gas phase in the vessel may be at least 30 mm/s. The sparger may be a sintered sparger or an orifice sparger. The thickness of the plate may be about 1 mm to about 25 mm. The accelerated liquid may have a velocity of about 8000 mm/s to about 17000 mm/s. In other examples, the accelerated liquid may have a velocity of about 12000 mm/s to about 17000 mm/s. In some examples, the bubbles injected into the liquid from the sparger may have a diameter of about 2 mm to about 20 mm. In another example, the bubbles injected into the liquid from the sparger may have a diameter of about 5 mm to about 15 mm, or from about 7 mm to about 13 mm. The fine bubbles may have a diameter of about 0.1 mm to about 5 mm, or about 0.2 mm to about 1.5 mm. The plurality of orifices may also be configured to accelerate at least 90% of the liquid in the vessel.

In another embodiment, the methods disclosed herein relate to generating fine bubbles that may include sparging gas into a vessel containing a liquid via at least one sparger positioned within the vessel and configured to inject bubbles into the liquid and accelerating a portion of the liquid in the vessel via a perforated plate positioned in an upper portion of the vessel, in which the liquid may be accelerated from the plate to break the bubbles into fine bubbles. In some examples, a superficial velocity of the gas phase in the vessel may be at least 30 mm/s. In other examples, the superficial velocity of the gas phase in the vessel may be from about 30 mm/s to about 80 mm/s. The sparger may be a sintered sparger or an orifice sparger. The liquid may be accelerated from the perforated plate at a velocity of about 8000 mm/s to about 17000 mm/s. In some examples, the liquid may be accelerated from the perforated plate at a velocity of about 12000 mm/s to about 17000 mm/s. The bubbles injected into the liquid from the sparger may have a diameter of about 2 mm to about 20 mm, or from greater than 5 mm to about 15 mm, or from about 7 mm to about 13 mm. Often the bubbles injected into the liquid from the sparger are not spherical. The injected bubbles may be referred to as coarse bubbles. In contrast, the fine bubbles may have a diameter of about 0.1 mm to about 5 mm, or about 0.2 mm to about 1.5 mm. The fine bubbles are typically spherical. The liquid stream may be introduced at a location proximate to the plate. The sparger may be positioned perpendicular or parallel to the plate, and a top or side surface of the sparger may be positioned from about 50 mm to about 300 mm, 500 mm, or 1000 mm from a bottom of the plate.

In yet another embodiment, the systems disclosed herein relate to a bioreactor that may include a vessel containing a liquid growth medium, a plate that may include a plurality of orifices positioned in an upper portion of the vessel and configured to accelerate at least a portion of the liquid growth medium in the vessel, a substrate that may include at least one C1 carbon source, at least one sparger positioned within the vessel with a surface of the sparger that may be positioned from about 50 mm to about 300 mm, 500 mm, or 1000 mm from a bottom of the plate and the sparger configured to inject substrate bubbles into the liquid growth medium. The sparger positioned within the vessel may create a first zone for the substrate bubbles to rise within the vessel, and a second zone for the accelerated liquid growth medium to break the substrate bubbles into substrate fine bubbles, and for fluid to flow through the vessel. The fluid may have the accelerated portion of the liquid growth medium and may have the substrate fine bubbles, and a culture of at least one microorganism in the liquid growth medium. The culture of at least one microorganism may anaerobically ferment the substrate to produce at least one fermentation product.

In still another embodiment, the methods disclosed herein relate to generating substrate fine bubbles in a bioreactor and may include sparging substrate bubbles of at least one C1 carbon source into a vessel containing a liquid growth medium via at least one sparger positioned within the vessel and accelerating a portion of the liquid growth medium in the vessel via a perforated plate positioned in an upper portion of the vessel. The liquid growth medium accelerated from the plate may break the substrate bubbles into substrate fine bubbles. A superficial velocity of the gas phase in the vessel may be at least 30 mm/s. A culture of at least one microorganism may be included in the liquid growth medium and may anaerobically ferment the substrate to produce at least one fermentation product.

Some embodiments are directed to the combination of a primary fermentation (first stage fermentation) that converts gas to acetate or ethanol, with a secondary fermentation (second stage fermentation) that converts acetate or ethanol to a target product.

### Primary Fermentation

The primary fermentation uses a microorganism (the “primary microorganism”) capable of converting gas to acetate or ethanol. The primary fermentation may be a co-culture of two such organisms, which have similar or complementary growth requirements. Thus, the primary fermentation may involve a pure culture or a mixed culture.

The primary microorganism may, for example, be selected from Acetobacterium, Acetoanaerobium, Alkalibaculum, Blautia, Butyribacterium, Clostridium, Desulfobacterium, Eubacterium, Methanosarcina, Moorella, Oxobacter, Peptostreptococcus, Pyrococcus Ruminococcus, Sporomusa, and Thermoanaerobacter. In particular, the primary microorganism may be derived from a parental bacterium selected from Acetobacterium woodii, Alkalibaculum bacchii, Blautia producta, Butyribacterium methylotrophicum, Carboxydothermus acetogenium, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui. The primary microorganism may also be selected from Acetitomaculum ruminis, Acetoanaerobium noterae, Acetobacterium bakii, Acetobacterium carbinolicum, Acetobacterium dehalogenans, Acetobacterium fimetarium, Acetobacterium malicum, Acetobacterium paludosum, Acetobacterium tundrae, Acetobacterium wieringae, Acetobacterium woodii, Acetohalobium arabicum, Acetonema longum, Blautia coccoides, Blautia hydrogenotrophica, Blautia producta, Blautia schinkii, Butyribacterium methylotrophicum, Clostridium aceticum, Clostridium autoethanogenum, Clostridium carboxidivorans, Clostridium drakei, Clostridium formicoaceticum, Clostridium glycolicum, Clostridium ljungdahlii, Clostridium magnum, Clostridium mayombei, Clostridium methoxybenzovorans, Clostridium ragsdalei, Clostridium scatologenes, Eubacterium aggregans, Eubacterium limosum, Moorella mulderi, Moorella thermoacetica, Moorella thermoautotrophica, Oxobacter pfennigii, Sporomusa acidovorans, Sporomusa aerivorans, Sporomusa malonica, Sporomusa ovata, Sporomusa paucivorans, Sporomusa rhizae, Sporomusa silvacetica, Sporomusa sphaeroides, Sporomusa termitida, Thermoacetogenium phaeum, Thermoanaerobacter kivui, Acetobacterium, Moorella, Moorella sp HUC22-1, Moorella thermoacetica, Clostridium, Clostridium carboxidivorans, Clostridium drakei, Clostridium acidi-urici, Pyrococcus, Pyrococcus furiosus, Eubacterium, Eubacterium limosum, Desulfobacterium, Carboxydothermus, Acetogenium, Acetoanaerobium, Butyribacterium, Butyribacterium methylotrophicum, Peptostreptococcus, Ruminococcus, Oxobacter, Oxobacter pfennigii, Methanosarcina, Carboxydothermus, Eubacterium limosum, Desulfotomaculum orientis, Peptococcus glycinophilus, Peptococcus magnus, Ignicoccus hospitalis, Thermoanaerobacter kivui, Cupriavidus necator, and Thermoacetogenium phaeum.

In certain embodiments, the primary fermentation is performed in the absence of carbohydrate substrates, such as sugar, starch, lignin, cellulose, or hemicellulose.

The primary fermentation produces at least one product (a “primary product”). Typically, this product will be acetate, although the primary fermentation may also produce additional products such as ethanol, lactate, and 2,3-butanediol. Microbial biomass, such as cultured protein or single cell protein, may also be considered a product, as it has potential applications in animal feed, food, probiotic, prebiotic, postbiotics, and fertilizers. Importantly, the terms “acetate” and “acetic acid” may be used interchangeably herein.

### Secondary Fermentation

The secondary fermentation uses a microorganism (the “secondary microorganism”) capable of converting acetate to a product. The secondary fermentation may be a co-culture of two such organisms, which have similar or complementary growth requirements. Thus, the primary fermentation may involve a pure culture or a mixed culture. The secondary fermentation may be conducted as an anaerobic or aerobic process.

The secondary microorganism may be any microorganism capable of consuming acetate or ethanol. The secondary microorganism may be a yeast or bacterial species. The secondary microorganism may be selected from those listed in the following Table 1:

TABLE 1

| | | Typical | | |
|---|---|---|---|---|
| | Used | fermentation | | |
| Exemplar species | industrially | type | Oxygenation | Known products |
| Escherichia coli | Yes | Batch | Any | Proteins, base chemicals, |
| | | | | fine chemicals |
| Yarrowia lipolytica | Yes | Continuous | Aerobic | Bulk and fine |
| | | | | [supplement] lipids |
| Saccharomyces | Yes | Batch | Anaerobic | Ethanol, dried distiller's |
| cerevisiae | | | | grains and solubles |
| Saccharomyces | Yes | Batch | Aerobic | Biomass, proteins, Base |
| cerevisiae | | | | Chemicals, fine |
| | | | | chemicals |
| Geobacillus | Yes | Batch | Aerobic | Enzymes [amylase] |
| thermoglucosidasius | | | | |
| Bacillus subtilis | Yes | | Aerobic | Enzymes |
| | | | | [decarboxylase, |
| | | | | glucanase, |
| | | | | hemicellulase, amylase, |
| | | | | xylanse], bacteriocins, |
| | | | | amino acids |
| Pseudomonas putida | No | Batch | Aerobic | Proteins, base chemicals, |
| KT2440 | | | | fine chemicals |
| Cryptococcus curvatus | No | Continuous | Aerobic | Lipids |
| Rhodotorula glutinis | No | Continuous | Aerobic | Lipids |
| Lipomyces starkeyi | No | Continuous | Aerobic | Lipids |
| Cupriavidus | No | Batch | Aerobic | Biomass, PHBs, PHAs, |
| necator/Ralstonia | | | | isobutanol |
| eutropha/Alcaligenes | | | | |
| eutrophus | | | | |
| Clostridium kluyveri | Yes | Continuous | Anaerobic | Butanoate, hexanoate, |
| | | | | octanoate, propionate |
| Methanosarcina | Yes | Continuous | Anaerobic | Methane, |
| acetivorans | | | | biomass/digestate |
| Desulfonatronum | No | Batch | Aerobic | N/A |
| cooperativum | | | | |
| Geoalkalibacter | No | Batch | Aerobic | N/A |
| ferrihydriticus | | | | |
| Alkalispirillum mobile | No | Batch | Aerobic | N/A |
| Corynebacterium | Yes | Batch | Aerobic | Amino acids, biomass, |
| glutamicum | | | | alcohols on research |
| | | | | scale |

The secondary microorganism is capable of consuming a substrate (a “secondary substrate”) that provides carbon and/or energy. Typically, the secondary substrate comprises the acetate or ethanol produced by the primary fermentation. However, the secondary substrate may also contain additional sources of carbon and/or energy, such as sugar or starch. In specific embodiments, acetate provides at least 50%, at least 60%, at least 70%, at least 80%, at least 90%, at least 95%, or at least 99% of the carbon and/or energy requirements of the secondary microorganism. In some embodiments, however, acetate or ethanol may be removed for another use and another substrate may drive the secondary fermentation.

The second fermentation produces at least one product, which may be referred to herein as a “target product.” The target product may be an fatty acid, fatty alcohol, enzyme, such as alpha acetolactate decarboxylase, alpha amylase, catalase, chymosin, cyclodextrin glucanotransferase, beta glucanase, glucose isomerase, glucose oxidase, hemicellulase, lipase, maltogenic amylase, protease, pullulanase, xylanase, or cellulase. The target product may be a nutrient or additive, such as microbial biomass for example, cultured protein or single cell protein), amino acids (glutamate, lysine, threonine, phenylalanine, tryptophan, cysteine, valine, proline, arginine, histidine), lipids, or vitamins (B12, B2, biotin). The target product may be a chemical such as butanoate, hexanoate, octanoate, isobutanol, isoprene, ethylene, or terpenes. The target product may also be, for example, methane, polyhydroxybutyrate, polyhydroxyalkanoate, 3-hydroxyproprionate, a C₅-C₂₀ alcohol, an organic acid, a ketone, a lipid, acetone, propanol, butanol, 2,3-butanediol, acetate, butyrate, caproate, propylene, butadiene, isobutylene, isobutyl acetate, succinate, mevalonate, itaconic acid, monellin, or ethylene. In some cases the target product may be just one or more of these products devoid of others, for example, devoid of lipids. In one embodiment, the target product may be polyhydroxybutyrate devoid of lipids. In another embodiment it may be polyhydroxyalkanoate devoid of lipids.

In some embodiments, fatty acids are selected from Hexanoic Acid, Octanoic Acid, Decanoic Acid, Dodecanoic Acid, Tetradecanoic Acid, Hexadecanoic Acid, Octadecanoic Acid, Palmitoleic acid (hexadec-9-enoic acid, cis- or trans-), Vaccenic acid (octadec-11-enoic acid, cis- or trans-), Octadec-9-enoic acid, cis- or trans-, 8-(2-hexylcyclopropyl)octanoic acid, 8-(2-octylcyclopropyl)octanoic acid, 10-(2-hexylcyclopropyl)decanoic acid, Additional general category: 3-hydroxy-Cₙ-acid (i.e., 3-hydroxy-tetradecanoic acid), Additional general category: Cₙ-2-enoic acid (i.e., tetradec-2-enoic acid), or any combination thereof.

In other embodiments, fatty acids are branched-chain acids selected from anteiso-C13:0 (anteisotridecanoic acid), iso-C13:0 (isotridecanoic acid), anteiso-C14:0 (anteisotetradecanoic acid), iso-C14:0 (isotetradecanoic acid), anteiso-C15:0 (anteisopentadecanoic acid), iso-C15:0 (isopentadecanoic acid), anteiso-C16:0 (anteisohexadecanoic acid), iso-C16:0 (isohexadecanoic acid), anteiso-C17:0 (anteisoheptadecanoic acid), iso-C17:0 (isoheptadecanoic acid), anteiso-C18:0 (anteisooctadecanoic acid), iso-C18:0 (isooctadecanoic acid), anteiso-C19:0 (anteisononadecanoic acid), iso-C19:0 (isononadecanoic acid), or any combination thereof.

In some embodiments, fatty alcohols are selected from Hexanol, Octanol, Decanol, Dodecanol, Tetradecanol, Hexadecanol, Octadecanol, Hexadec-9-en-1-ol, cis- or trans-, Octadec-11-en-1-ol, cis- or trans-, Octadec-9-en-1-ol, Additional general category: 3-hydroxy-Cₙ-1-ol (i.e., 3-hydroxy-tetradecanol), Additional general category: Cₙ-2-en-1-ol (i.e., tetradec-2-enol), or any combination thereof.

In other embodiments, fatty alcohols are branched-chain fatty alcohols selected from anteiso-C13:0 (anteisotridecanol), iso-C13:0 (isotridecanol), anteiso-C14:0 (anteisotetradecanol), iso-C14:0 (isotetradecanol), anteiso-C15:0 (anteisopentadecanol), iso-C15:0 (isopentadecanol), anteiso-C16:0 (anteisohexadecanol), iso-C16:0 (isohexadecanol), anteiso-C17:0 (anteisoheptadecanol), iso-C17:0 (isoheptadecanol), anteiso-C18:0 (anteisooctadecanol), iso-C18:0 (isooctadecanol), or any combination thereof.

In an embodiment, the fatty acid is converted into a product selected from soaps, hand soap, face soap, bar soap, shampoo, lotions, creams, facial toners, sanitary cleaners, dish soap, laundry detergent, cleaning powders, lubricant, metal, plastic, paints, wood coatings, metal coatings, plastic surfaces, vegetable-based plastic additives, animal feed, animal feed additives, emulsifiers, emulsifier agents, food emulsifiers, suspension agents, food thickeners, or any combination thereof.

In some embodiment, the fatty alcohol is converted into a product selected from foods, personal care items, pharmaceutical, emollients, additives, surfactants, cosmetics, foundations, concealers, lipsticks, shampoo, conditioner, salt scrubs, skincare products, lotions, gels, moisturizers, fragrances, perfumes, colognes, lubricants, or any combination thereof.

In some embodiments the culture protein is used in protein-rich snacks: single-cell proteins can be used to create protein-rich snacks like protein bars, protein crisps, or protein cookies.

In other embodiment the cultured protein is used in meat substitutes: single-cell proteins can be used to develop meat substitutes like plant-based burgers, sausages, or nuggets that offer a protein-rich alternative to traditional meat products.

In an embodiment the cultured protein is used in dairy alternatives: single-cell proteins can be utilized in the production of dairy alternatives such as plant-based milks (e.g., almond milk, oat milk, or soy milk) and non-dairy yogurts.

In an embodiment the cultured protein is used in fermented foods: single-cell proteins can be employed in the fermentation process of various foods like tempeh, miso, or soy sauce, providing a protein boost to these products.

In an embodiment the cultured protein is used in protein beverages: single-cell proteins can be incorporated into protein beverages such as protein shakes, smoothies, or sports drinks, offering a convenient and nutritious source of protein.

In an embodiment the cultured protein is used in baked goods: single-cell proteins can be used as an ingredient in baked goods like bread, cakes, or pastries, enhancing their protein content.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

These and other aspects of the present disclosure, which should be considered in all its novel aspects, will become apparent from the following description, which is given by way of example only, with reference to the accompanying figures, in which:

FIG. 1 shows pathways for the production of C6-C18 fatty acids and fatty alcohols from the central carbon intermediates acetyl-CoA exploiting the ability for acyl-ACP:CoA transacylase to convert ACP intermediates into CoA intermediates.

FIG. 2 compares heterotrophic fatty alcohol production under nitrogen-limited conditions by Cupriavidus necator PHB-4 strains transformed with pBBR1 plasmids expressing either Marinobacter sp. BSs20148 acyl-ACP reductase (acyl-ACP reductase route) or the combination of Pseudomonas koreensis 3-hydroxyacyl-ACP:CoA transacylase, Treponema denticola trans-enoyl-CoA reductase, and Marinobacter hydrocarbonoclasticus VT8 acyl-CoA reductase (acyl-ACP:CoA transacylase route). Bars represent fatty alcohols contained in the 20% dodecane overlay, with concentration normalized to the aqueous volume.

FIG. 3 shows total autotrophic total C6-C16 fatty alcohol production under nitrogen-limited conditions by Cupriavidus necator PHB-4 transformed with pBBR1 plasmid expressing Pseudomonas koreensis 3-hydroxyacyl-ACP:CoA transacylase, Treponema denticola trans-enoyl-CoA reductase, and Marinobacter hydrocarbonoclasticus VT8 acyl-CoA reductase driven by nitrogen-limited inducible promoter.

FIG. 4 shows the carbon chain length distribution of autotrophic fatty alcohol and acid production at day 5 of the time course shown in FIG. 3.

FIG. 5 compares heterotrophic fatty acid production under nitrogen-limited conditions by Cupriavidus necator PHB-4 strains transformed with pBBR1 plasmids expressing either E. coli thioesterase TesA′ and Treponema denticola trans-enoyl-CoA reductase (EcTesA+TdTER) or the combination of Pseudomonas koreensis 3-hydroxyacyl-ACP:CoA transacylase, Treponema denticola trans-enoyl-CoA reductase, and E. coli thioesterase TesA′ (EcTesA+TdTER+PkorPhaG). Bars represent the concentrations of fatty acids contained in the 20% dodecane overlay.

FIG. 6 compares heterotrophic fatty acid production under nitrogen-limited conditions by Cupriavidus necator PHB-4 strains transformed with pBBR1 plasmids expressing either E. coli thioesterase FadM and Treponema denticola trans-enoyl-CoA reductase (EcFadM+TdTER) or the combination of Pseudomonas koreensis 3-hydroxyacyl-ACP:CoA transacylase, Treponema denticola trans-enoyl-CoA reductase, and E. coli thioesterase FadM (EcFadM+TdTER+PkorPhaG). Bars represent the concentrations of fatty acids contained in the 20% dodecane overlay.

### DETAILED DESCRIPTION

The following description of embodiments is given in general terms. The disclosure is further elucidated from the disclosure given under the heading “Examples” herein below, which provides experimental data supporting the disclosure, specific examples of various aspects of the disclosure, and means of performing the disclosure.

The inventors have surprisingly been able to engineer a C1-fixing microorganism to co-produce a protein, a chemical or a precursor of the chemical, and microbial biomass by fermentation of a substrate comprising CO and/or CO₂.

Unless otherwise defined, the following terms as used throughout this specification are defined as follows:

The disclosure provides microorganisms for the biological co-production of proteins, chemicals, and microbial biomass. A “microorganism” is a microscopic organism, especially a bacterium, archaeon, virus, or fungus. In an embodiment, the microorganism of the disclosure is a bacterium.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility. The microorganisms of the disclosure are non-naturally occurring.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization. The microorganisms of the disclosure are genetically engineered.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms. The microorganisms of the disclosure are generally recombinant.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure.

Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

In particular, a heterologous nucleic acid or protein expressed in the microorganism described herein may be derived from Bacillus, Clostridium, Cupriavidus, Escherichia, Gluconobacter, Hyphomicrobium, Lysinibacillus, Paenibacillus, Pseudomonas, Sedimenticola, Sporosarcina, Streptomyces, Thermithiobacillus, Thermotoga, Zea, Klebsiella, Mycobacterium, Salmonella, Mycobacteroides, Staphylococcus, Burkholderia, Listeria, Acinetobacter, Shigella, Neisseria, Bordetella, Streptococcus, Enterobacter, Vibrio, Legionella, Xanthomonas, Serratia, Cronobacter, Cupriavidus, Helicobacter, Yersinia, Cutibacterium, Francisella, Pectobacterium, Arcobacter, Lactobacillus, Shewanella, Erwinia, Sulfurospirillum, Peptococcaceae, Thermococcus, Saccharomyces, Pyrococcus, Glycine, Homo, Ralstonia, Brevibacterium, Methylobacterium, Geobacillus, Bos, gallus, Anaerococcus, Xenopus, Amblyrhynchus, Rattus, Mus, Sus, Rhodococcus, Rhizobium, Megasphaera, Mesorhizobium, Peptococcus, Agrobacterium, Campylobacter, Acetobacterium, Alkalibaculum, Blautia, Butyribacterium, Eubacterium, Moorella, Oxobacter, Sporomusa, Thermoanaerobacter, Schizosaccharomyces, Paenibacillus, Fictibacillus, Lysinibacillus, Ornithinibacillus, Halobacillus, Kurthia, Lentibacillus, Anoxybacillus, Solibacillus, Virgibacillus, Alicyclobacillus, Sporosarcina, Salimicrobium, Sporosarcina, Planococcus, Corynebacterium, Thermaerobacter, Sulfobacillus, or Symbiobacterium.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “exogenous gene product” are used herein to refer to a protein molecule that is the product of the expression of an exogenous gene.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

The term “copolymer” is a composition comprising two or more species of monomers are linked in the same polymer chain of the disclosure.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

“Disrupted gene” refers to a gene that has been modified in some way to reduce or eliminate expression of the gene, regulatory activity of the gene, or activity of an encoded protein or enzyme. The disruption may partially inactivate, fully inactivate, or delete the gene or enzyme. The disruption may be a knockout (KO) mutation that fully eliminates the expression or activity of a gene, protein, or enzyme. The disruption may also be a knock-down that reduces, but does not entirely eliminate, the expression or activity of a gene, protein, or enzyme. The disruption may be anything that reduces, prevents, or blocks the biosynthesis of a product produced by an enzyme. The disruption may include, for example, a mutation in a gene encoding a protein or enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, RNAi, TALEN, siRNA, CRISPR, or CRISPRi) or protein which inhibits the expression of a protein or enzyme. The disruption may be introduced using any method known in the art. For the purposes of the present disclosure, disruptions are laboratory-generated, not naturally occurring.

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes.

The microorganism of the disclosure may be further classified based on functional characteristics. For example, the microorganism of the disclosure may be or may be derived from a C1-fixing microorganism, an anaerobe, an acetogen, an ethanologen, a carboxydotroph, an autotroph, and/or a methanotroph. The microorganism of the disclosure may be selected from chemoautotroph, hydrogenotroph, knallgas, methanotroph, or any combination thereof. In some embodiments, the microorganism may be hydrogen-oxidizing, carbon monoxide-oxidizing, knallgas, or any combination thereof, with the capability to grow and synthesize biomass on gaseous carbon sources such as syngas and/or CO₂, such that the production microorganisms synthesize targeted chemical products under gas cultivation. The microorganisms and methods of the present disclosure can enable low cost synthesis of biochemicals, which can compete on price with petrochemicals and higher-plant derived amino acids, proteins, and other biological nutrients. In certain embodiments, these amino acids, proteins, and other biological nutrients are predicted to have a substantially lower price than amino acids, proteins, and other biological nutrients produced through heterotrophic or microbial phototrophic synthesis. Knallgas microbes, hydrogenotrophs, carboxydotrophs, and chemoautotrophs more broadly, are able to capture CO₂ or CO as their sole carbon source to support biological growth. In some embodiments, this growth includes the biosynthesis of amino acids and proteins. Knallgas microbes and other hydrogenotrophs can use H₂ as a source of reducing electrons for respiration and biochemical synthesis. In some embodiments of the present invention knallgas organisms and/or hydrogenotrophs and/or carboxydotrophs and/or other chemoautotrophic microorganisms are grown on a stream of gasses including but not limited to one or more of the following: CO₂; CO; H₂; along with inorganic minerals dissolved in aqueous solution. In some embodiments knallgas microbes and/or hydrogenotrophs and/or carboxydotrophs and/or other chemoautotrophic and/or methanotrophic microorganisms convert greenhouse gases into biomolecules including amino acids and proteins.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1-carbon source. Often, the microorganism of the disclosure is a C1-fixing bacterium.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen), sometimes referred to as “microoxic conditions.” Often, the microorganism of the disclosure is an anaerobe. In a preferred embodiment, the microorganism of the disclosure is derived from an anaerobe identified in Table 1.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, NY, 2006). All naturally occurring acetogens are C1-fixing, anaerobic, autotrophic, and non-methanotrophic.

An “ethanologen” is a microorganism that produces or is capable of producing ethanol. Often, the microorganism of the disclosure is an ethanologen. In a preferred embodiment, the microorganism of the disclosure is derived from an ethanologen identified in Table 1.

An “autotroph” is a microorganism capable of growing in the absence of organic carbon. Instead, autotrophs use inorganic carbon sources, such as CO and/or CO₂. Often, the microorganism of the disclosure is an autotroph. In a preferred embodiment, the microorganism of the disclosure is derived from an autotroph identified in Table 1.

A “carboxydotroph” is a microorganism capable of utilizing CO as a sole source of carbon and energy. Often, the microorganism of the disclosure is a carboxydotroph. In a preferred embodiment, the microorganism of the disclosure is derived from a carboxydotroph identified in Table 1.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP).

The terms “oxyhydrogen” and “oxyhydrogen microorganism” can be used synonymously with “knallgas” and “knallgas microorganism” respectively. Knallgas microorganisms generally use molecular hydrogen by means of hydrogenases, with some of the electrons donated from H₂ being utilized for the reduction of NAD⁺ (and/or other intracellular reducing equivalents) and some of the electrons from H₂ being used for aerobic respiration. Knallgas microorganisms generally fix CO₂ autotrophically, through pathways including but not limited to the Calvin Cycle or the reverse citric acid cycle.

In another embodiment, the microorganism of the disclosure is an aerobic bacterium. In one embodiment, the microorganism of the disclosure comprises aerobic hydrogen bacteria. In an embodiment, the aerobic bacteria comprising at least one disrupted gene.

A number of aerobic bacteria are known to be capable of carrying out fermentation for the disclosed methods and system. Examples of such bacteria that are suitable for use in the invention include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

In some embodiments, the microorganism is Cupriavidus necator DSM248, DSM540, DSM541 or DSM34774.

For the purposes of the present disclosure, the strain of C. necator may be modified by, for example, adaptive laboratory evolution or genetic modification.

For the purposes of the present disclosure, the strain of C. necator may be a strain that grows autotrophically at up to 40° C. For example, the strain of C. necator may be the strain deposited at DSM34774. Additionally or alternatively, the strain of C. necator may have a H2:CO2 uptake ratio that is lower than the H2:CO2 uptake ratio of a wild type or naturally occurring strain of C. necator cultured under corresponding temperatures and conditions. In some embodiments, the strain of C. necator may comprise a full or partial deletion of a gene or locus encoding a membrane-bound hydrogenase, such as hoxKGXZ.

In some embodiments, the aerobic bacteria comprises one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or any combination thereof.

In the microorganisms of the disclosure, carbon flux is strategically diverted away from nonessential or undesirable products and towards products of interest. In certain embodiments, these disrupted genes divert carbon flux away from nonessential or undesirable metabolic nodes and through target metabolic nodes to improve production of products downstream of those target metabolic nodes. In an embodiment, limitation selected from nutrients, dissolved oxygen, or any combination thereof diverts carbon flux to desired products.

The C. necator strain having identification reference number DSM 34774 was deposited under the provisions of the Budapest Treaty at the German Collection of Microorganisms (DSM) located at Inhoffenstraße 7B, 38124 Braunschweig, Science Campus Braunschweig-Süd, Germany on Oct. 6, 2023. All restrictions on the availability to the public of the deposited material will be irrevocably removed upon the granting of a patent from the above-identified application. The deposited cultures will be replaced should they die or be destroyed during the enforceable life of any patent issued out of this patent application, for five years after the last request for a sample of the deposited microorganism or for a term of at least thirty (30) years. Samples will be stored under agreements that would make them available beyond the enforceable life of the patent for which the deposit was made.

Additionally or alternatively, the strain of C. necator utilized for the present disclosure may be modified to possess one or more other desirable traits. For example, the strain of C. necator may be modified either via genetic modification or ALE to minimize the H2:CO2 uptake ratio while maximizing intracellular O2 during autotrophic growth. This can be done through the removal or reduction in a membrane-bound hydrogenase system. This can aid in reducing the cost of hydrogen associated with commercial-scale gas fermentation.

A strain of C. necator can be modified to minimize the H2:CO2 uptake ratio by deleting all or a portion of, or otherwise reducing the expression of a membrane-bound hydrogenase system (e.g., the hoxKGZ locus). The hoxKGZ locus encodes a multi-subunit membrane-bound hydrogenase system in Cupriavidus necator, and deleting this locus does not inhibit C. necator autotrophic growth on a gas mixture composed of hydrogen, oxygen, and carbon dioxide. Without being bound by theory, this may result because while the soluble hydrogenase system is required for autotrophic growth, the membrane-bound hydrogenase system wastefully oxidizes hydrogen gas, increasing the culture's H2:CO2 uptake ratio without benefiting growth. By deleting or reducing expression of the hoxKGZ locus, growth is not only permitted autotrophically, but the H2:CO2 uptake ratio is also reduced while increasing the intracellular 02 availability to improve 02-dependent product synthesis.

In one embodiment, the aerobic bacteria may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, isoamyl alcohol, methacrolein, methyl-methacrylate, or any combination thereof.

In another embodiment, the bacteria of the disclosure may produce ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, keto-adipic acid, 1,3hexanediol, 3-methyl-2-butanol, 2-buten-1-ol, isovalerate, isoamyl alcohol, and monoethylene glycol, or any combination thereof.

The disclosure provides microorganisms capable of producing ethylene comprising culturing the microorganism of the disclosure in the presence of a substrate, whereby the microorganism produces ethylene.

The enzymes of the disclosure may be codon optimized for expression in the microorganism of the disclosure. “Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the disclosure are codon optimized for expression in the microorganism of the disclosure. Although codon optimization refers to the underlying genetic sequence, codon optimization often results in improved translation and, thus, improved enzyme expression. Accordingly, the enzymes of the disclosure may also be described as being codon optimized.

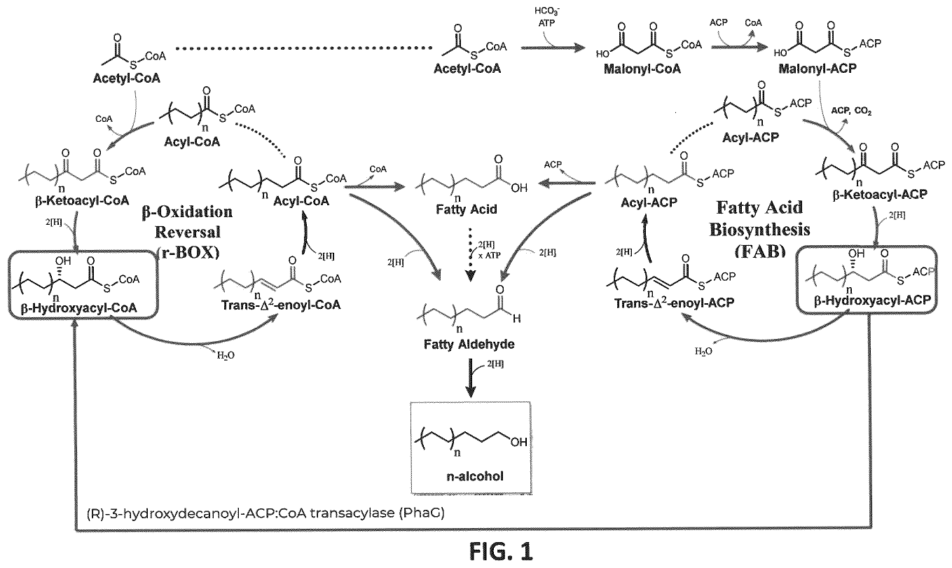

One or more of the enzymes of the disclosure may be overexpressed. “Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate. As described above, one or more of the enzymes catalyzing reactions 2, 5, 6, 8, 9, 10, 19, 20, 24, or 25 of FIG. 1 may be overexpressed.

The enzymes of the disclosure may comprise a disruptive mutation. A “disruptive mutation” refers to a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

Although exemplary sequences and sources for enzymes are provided herein, the disclosure is by no means limited to these sequences and sources—it also encompasses variants. The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In a preferred embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate ferredoxin oxidoreductase promoter, an Rnf complex operon promoter, an ATP synthase operon promoter, or a phosphotransacetylase/acetate kinase operon promoter.

It should be appreciated that the disclosure may be practiced using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants.” By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein.

The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the disclosure may be practiced using polypeptides whose sequence varies from the amino acid sequences specifically exemplified herein. These variants may be referred to herein as “functionally equivalent variants.” A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

The microorganisms of the disclosure may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the disclosure is produced by a method comprises the following steps: introduction into a shuttle microorganism of (i) of an expression construct/vector as described herein and (ii) a methylation construct/vector comprising a methyltransferase gene; expression of the methyltransferase gene; isolation of one or more constructs/vectors from the shuttle microorganism; and, introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism that facilitates the methylation of the nucleic acid sequences that make up the expression construct/vector. In a particular embodiment, the shuttle microorganism is a restriction negative E. coli, Bacillus subtilis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the disclosure, the methylation construct/vector comprises an inducible lac promoter and is induced by addition of lactose or an analogue thereof, more preferably isopropyl-β-D-thiogalactoside (IPTG). Other suitable promoters include the ara, tet, or T7 system. In a further embodiment of the disclosure, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the disclosure.

In one particular embodiment, the expression construct/vector and/or the methylation construct/vector are plasmids.

Persons of ordinary skill in the art will appreciate a number of suitable methyltransferases of use in producing the microorganisms of the disclosure. However, by way of example the Bacillus subtilis phage ΦT1 methyltransferase and the methyltransferase described in the Examples herein after may be used. Nucleic acids encoding suitable methyltransferases will be readily appreciated having regard to the sequence of the desired methyltransferase and the genetic code.

Any number of constructs/vectors adapted to allow expression of a methyltransferase gene may be used to generate the methylation construct/vector.

In one embodiment, the substrate comprises CO. In one embodiment, the substrate comprises CO₂ and CO. In another embodiment, the substrate comprises CO₂ and H₂. In another embodiment, the substrate comprises CO₂ and CO and H₂.

“Substrate” refers to a carbon and/or energy source for the microorganism of the disclosure. Often, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14: 127, 2015). In some embodiments, the substrate does not comprise a pentose such as xylose (Pereira, Metab Eng, 34: 80-87, 2016). In some embodiments, the substrate may comprise both gaseous and carbohydrate substrates (mixotrophic fermentation). The substrate may further comprise other non-carbon components, such as H₂, N₂, or electrons.

The gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H₂. For example, the gaseous substrate may comprise about 1, 2, 5, 10, 15, 20, or 30 mol % H₂. In some embodiments, the gaseous substrate may comprise a relatively high amount of H₂, such as about 60, 70, 80, or 90 mol % H₂. In further embodiments, the gaseous substrate comprises no or substantially no (<1 mol %) H₂.

The gaseous substrate may comprise some amount of CO₂. For example, the gaseous substrate may comprise about 1-80 or 1-30 mol % CO₂. In some embodiments, the gaseous substrate may comprise less than about 20, 15, 10, or 5 mol % CO₂. In another embodiment, the gaseous substrate comprises no or substantially no (<1 mol %) CO₂.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof.

Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

In certain embodiments, the fermentation is performed in the absence of carbohydrate substrates, such as sugar, starch, fiber, lignin, cellulose, or hemicellulose.

In addition to tandem repeat proteins and chemical products, the microorganism of the disclosure may be cultured to produce one or more co-products. For instance, the microorganism of the disclosure may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and/or monoethylene glycol (WO 2019/126400) in addition to ethylene. In certain embodiments, microbial biomass itself may be considered a product. These products may be further converted to produce at least one component of diesel, jet fuel, sustainable aviation fuel (SAF), and/or gasoline. In certain embodiments, ethylene may be catalytically converted into another product, article, or any combination thereof. Additionally, the microbial biomass may be further processed to produce a cultured protein or single cell protein (SCP) by any method or combination of methods known in the art. In addition to one or more target chemical products, the microorganism of the disclosure may also produce ethanol, acetate, and/or 2,3-butanediol. In another embodiment, the microorganism and methods of the disclosure improve the production of products, proteins, microbial biomass, or any combination thereof.

A “native product” is a product produced by a genetically unmodified microorganism. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product, such as ethylene glycol, accounts for at least about 5%, 10%, 15%, 20%, 30%, 50%, or 75% of all fermentation products produced by the microorganism of the disclosure. In one embodiment, ethylene accounts for at least 10% of all fermentation products produced by the microorganism of the disclosure, such that the microorganism of the disclosure has a selectivity for ethylene glycol of at least 10%. In another embodiment, ethylene accounts for at least 30% of all fermentation products produced by the microorganism of the disclosure, such that the microorganism of the disclosure has a selectivity for ethylene of at least 30%.

At least one of the one or more fermentation products may be biomass produced by the culture. At least a portion of the microbial biomass may be converted to a cultured protein or single cell protein (SCP). At least a portion of the cultured protein or single cell protein may be utilized as a component of animal feed.

In one embodiment, the disclosure provides an animal feed comprising microbial biomass and at least one excipient, wherein the microbial biomass comprises a microorganism grown on a gaseous substrate comprising one or more of CO, CO₂, and H₂.

A “cultured protein” or “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

“Microbial biomass” refers biological material comprising microorganism cells. For example, microbial biomass may comprise or consist of a pure or substantially pure culture of a bacterium, archaea, virus, or fungus. When initially separated from a fermentation broth, microbial biomass generally contains a large amount of water. This water may be removed or reduced by drying or processing the microbial biomass.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavour, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3rd revision, pages 575-633, 2014.

A “biopolymer” refers to natural polymers produced by the cells of living organisms. In certain embodiments, the biopolymer is PHA. In certain embodiments, the biopolymer is PHB.

A “bioplastic” refers to plastic materials produced from renewable biomass sources. A bioplastic may be produced from renewable sources, such as vegetable fats and oils, corn starch, straw, woodchips, sawdust, or recycled food waste.

Herein, reference to an acid (e.g., acetic acid or 2-hydroxyisobutyric acid) should be taken to also include the corresponding salt (e.g., acetate or 2-hydroxyisobutyrate).

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of ethylene glycol. If necessary, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

A “sparger” may comprise a device to introduce gas into a liquid, injected as bubbles, to agitate it or to dissolve the gas in the liquid. Example spargers may include orifice spargers, sintered spargers, and drilled pipe spargers. In certain configurations drilled pipe spargers may be mounted horizontally. In other examples, spargers may be mounted vertically or horizontally. In some examples, the sparger may be a perforated plate or ring, sintered glass, sintered steel, porous rubber pipe, porous metal pipe, porous ceramic or stainless steel, drilled pipe, stainless steel drilled pipe, polymeric drilled pipe, etc. The sparger may be of various grades (porosities) or may include certain sized orifices to produce a specific sized bubble or range of bubble sizes.

A “vessel”, “reaction vessel”, or “column” may be a vessel or container in which one or more gas and liquid streams, or flows may be introduced for bubble generation and/or fine bubble generation, and for subsequent gas-liquid contacting, gas-absorption, biological or chemical reaction, or surface-active material adsorption. In a reaction vessel, the gas and liquid phases may flow in the vertical directions. In a reaction vessel, larger bubbles from a sparger, having a buoyancy force larger than the drag force imparted by the liquid, may rise upwards. Smaller fine bubbles, having a buoyancy force less than or equal to the drag force imparted by the liquid, may flow downward with the liquid, as described by the systems and methods disclosed herein. A column or reaction vessel may not be restricted to any specific aspect (height to diameter) ratio. A column or reaction vessel may also not be restricted to any specific material and can be constructed from any material suitable to the process such as stainless steel, PVC, carbon steel, or polymeric material. A column or reaction vessel may contain internal components such as one or more static mixers that are common in biological and chemical engineering processing. A reaction vessel may also consist of external or internal heating or cooling elements such as water jackets, heat exchangers, or cooling coils. The reaction vessel may also be in fluid contact with one or more pumps to circulate liquid, bubbles, fine bubbles, and or one or more fluids of the system.

A “perforated plate” or “plate” may comprise a plate or similar arrangement designed to facilitate the introduction of liquid or additional liquid into the vessel that may be in the form of multiple liquid jets (i.e., accelerated liquid flow). The perforated plate may have a plurality of pores or orifices evenly or unevenly distributed across the plate that allow the flow of liquid from a top of the plate to the bottom of the plate. In some examples, the orifices may be spherical-shaped, rectangular-shaped, hexagonal prism-shaped, conical-shaped, pentagonal prism-shaped, cylindrical-shaped, frustoconical-shaped, or round-shaped. In other examples, the plate may comprise one or more nozzles adapted to generate liquid jets which flow into the column. The plate may also contain channels in any distribution or alignment where such channels are adapted to receive liquid and facilitate flow through into the reaction vessel. The plate may be made of stainless steel with a predefined number of laser-burnt, machined, or drilled pores or orifices. The specific orifice size may depend upon the required fine bubble size and required liquid, fine bubble, and/or fluid velocities. A specific orifice shape may be required to achieve the proper liquid acceleration and velocity from the plate to break or shear the sparger bubbles into the desired fine bubble size, and to create enough overall fluid downflow to carry the fine bubbles and liquid downward in the reaction vessel. The shape of the orifice may also impact ease of manufacturing and related costs. According to one embodiment, a straight orifice may be optimal due to ease of manufacture.

The systems and methods as disclosed herein, employ, within a vessel, multiple liquid jets or portions of accelerated liquid flow generated using the perforated plate to accelerate liquid and break bubbles into smaller fine bubbles having a greater superficial surface area than the original bubbles. The original bubbles are initially generated by injecting gas with a sparger positioned entirely within the reaction vessel. In one example, original bubbles injected into liquid from a sparger may have a diameter of about 2 mm to about 20 mm. In another example, original bubbles injected into liquid from a sparger may have a diameter of about 5 mm to about 15 mm. In other examples, original bubbles injected into liquid from a sparger may have a diameter of about 7 mm to about 13 mm. Upon injection, the original bubbles subsequently migrate upwards through the liquid and encounter the multiple liquid jets or portions of accelerated liquid flow which breaks the original bubbles into fine bubbles. The resulting fine bubbles and liquid flow down the reactor vessel in the downward fluid flow. The fine bubbles of substrate provide a carbon source and optionally an energy source to the microbes which then produce one or more desired products. The spargers are positioned within the vessel to create a first zone for the original bubbles to rise within the vessel, and to create a second zone for the accelerated liquid to break the original bubbles into fine bubbles and for fluid to flow through the vessel, where the fluid comprises the accelerated portion of the liquid and fine bubbles.

Due to the nature of the multi-phase system, one approach to maximizing product generation is to increase gas to liquid mass transfer. The more gas substrate transferred to a reaction liquid, the greater the desired product generated. The smaller fine bubbles of the present disclosure provide an increased superficial surface area resulting in an increased gas to liquid mass transfer rates overcoming known solubility issues. Additionally, the downflow reactor systems disclosed herein are effective to increase the residence time of the fine bubbles. The increased time that the fine bubbles remain in the reaction liquid generally provides increased amounts of reaction product generated, as well as greater surface areas in contact with the microbes. As such, the systems and methods disclosed herein improve over previous systems by generating fine bubbles that maximize gas to liquid superficial surface areas leading to high gas to liquid mass transfer rates. Further, the systems and methods disclosed herein provide superficial gas and liquid velocities not achieved by the previous systems and methods resulting in the generation of fine bubbles with high gas phase residence time resulting in the efficient creation of chemical and biological reaction products.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor. Purification techniques may include affinity tag purification (e.g. His, Twin-Strep, and FLAG), bead-based systems, a tip-based approach, and FPLC system for larger scale, automated purifications. Purification methods that do not rely on affinity tags (e.g. salting out, ion exchange, and size exclusion) are also disclosed.

In some embodiments, the produced chemical product may be isolated and enriched, including purified, using any suitable separation and/or purification technique known in the art. In an embodiment, the produced chemical product is gaseous. In one embodiment, the chemical product is a liquid. In an embodiment, a gaseous chemical product may pass a filter, a gas separation membrane, a gas purifier, or any combination thereof. In one embodiment, the chemical product is separated by an absorbent column. In another embodiment, the chemical product is stored in one or more cylinders after separation. In one embodiment, the chemical product is integrated into an infrastructure or process of an oil, gas, refinery, petrochemical operation, or any combination thereof. The infrastructure or process may be existing or new. In an embodiment, the gas fermentation product is integrated into oil and gas production, transportation and refining, and/or chemical complexes. In another embodiment, the source of the feedstock is from an oil, gas, refinery, petrochemical operation, or any combination thereof. In an embodiment, the gas fermentation product is integrated into an infrastructure or process of an oil, gas, refinery, petrochemical operation, or any combination thereof, and the source of the feedstock is from an oil, gas, refinery, petrochemical operation, or any combination thereof.

In some embodiments, distillation may be employed to purify a product gas. In an embodiment, gas-liquid extraction may be employed. In an embodiment, a liquid product isolation may also be enriched via extraction using an organic phase. In another embodiment, purification may involve other standard techniques selected from ultrafiltration, one or more chromatographic techniques, or any combination thereof.

The method of the disclosure may further comprise separating a gas fermentation product from the fermentation broth. The gas fermentation product may be separated or purified from a fermentation broth using any method or combination of methods known in the art, including, for example, distillation, simulated moving bed processes, membrane treatment, evaporation, pervaporation, gas stripping, phase separation, ion exchange, or extractive fermentation, including for example, liquid-liquid extraction. As described in U.S. Pat. No. 2,769,321, the disclosure of which is incorporated by reference in its entirety herein, ethylene may be separated according to the method or combination of methods known in the art. In one embodiment, the ethylene produced is harvested from the bioreactor culture vessel.

In one embodiment, the gas fermentation product may be concentrated from the fermentation broth using reverse osmosis and/or pervaporation (U.S. Pat. No. 5,552,023). Water may be removed by distillation and the bottoms (containing a high proportion of gas fermentation product) may then be recovered using distillation or vacuum distillation to produce a high purity stream. Alternatively, with or without concentration by reverse osmosis and/or pervaporation, the gas fermentation product may be further purified by reactive distillation with an aldehyde (Atul, Chem Eng Sci, 59: 2881-2890, 2004) or azeotropic distillation using a hydrocarbon (U.S. Pat. No. 2,218,234). In another approach, the gas fermentation product may be trapped on an activated carbon or polymer absorbent from aqueous solution (with or without reverse osmosis and/or pervaporation) and recovered using a low boiling organic solvent (Chinn, Recovery of Glycols, Sugars, and Related Multiple —OH Compounds from Dilute-Aqueous Solution by Regenerable Adsorption onto Activated Carbons, University of California Berkeley, 1999). The gas fermentation product can then be recovered from the organic solvent by distillation. In certain embodiments, the gas fermentation product is recovered from the fermentation broth by continuously removing a portion of the broth from the bioreactor, separating microbial cells from the broth (conveniently by filtration), and recovering the gas fermentation product from the broth. Co-products, such as alcohols or acids may also be separated or purified from the broth. Alcohols may be recovered, for example, by distillation. Acids may be recovered, for example, by adsorption on activated charcoal. Separated microbial cells may be returned to the bioreactor in certain embodiments. Further, separated microbial cells may be recycled to the bioreactor in some embodiments. The cell-free permeate remaining after target products have been removed is also preferably returned to the bioreactor, in whole or in part. Additional nutrients (such as B vitamins) may be added to the cell-free permeate to replenish the medium before it is returned to the bioreactor.

Recovery of diols from aqueous media has been demonstrated a number of ways. Simulated moving bed (SMB) technology has been used to recover 2,3-butaendiol from an aqueous mixture of ethanol and associated oxygenates (U.S. Pat. No. 8,658,845). Reactive separation has also been demonstrated for effective diol recovery. In some embodiments, recovery of ethylene glycol is conducted by reaction of the diol-containing stream with aldehydes, fractionation and regeneration of the diol, final fractionation to recover a concentrated diol stream. See, e.g., U.S. Pat. No. 7,951,980.

In one embodiment, the method comprises recovering ethylene produced as disclosed above. In one embodiment, the method further comprises converting or using ethylene in the production of one or more chemical products following recovery of ethylene.

Ethylene is a high value gaseous compound which is widely used in industry. In an embodiment, ethylene may be used as an anaesthetic or as a fruit ripening agent, as well as in the production of a number of other chemical products. In some embodiments, ethylene may be used to produce polyethylene and other polymers, such as styrene, polystyrene, ethylene oxide, ethylene dichloride, ethylene dibromide, ethyl chloride and ethylbenzene. Ethylene oxide is, for example, a key raw material in the production of surfactants and detergents and in the production of ethylene glycol, which is used in the automotive industry as an antifreeze product. In one embodiment directed to ethylene dichloride, ethylene dibromide, and ethyl chloride may be used to produce products such as polyvinyl chloride, trichloroethylene, perchloroethylene, methyl chloroform, polyvinylidene chloride and copolymers, and ethyl bromide. In an embodiment, ethylbenzene is a precursor to styrene, which is used in the production of polystyrene (used as an insulation product) and styrene-butadiene (which is rubber suitable for use in tires and footwear). In another embodiment, a product is an ethylene propylene diene monomer (EPDM) rubber, an ethylene propylene (EPR/EPM) rubber, or any combination thereof.

It should be appreciated that the methods of the invention may be integrated or linked with one or more methods for the production of downstream chemical products from ethylene. In some embodiments, the methods of the invention may feed ethylene directly or indirectly to chemical processes or reactions sufficient for the conversion or production of other useful chemical products.

In some embodiments, ethylene is converted into hydrocarbon liquid fuels. In an embodiment, ethylene is oligomerized over a catalyst to selectively produce target products selected from gasoline, condensate, aromatics, heavy oil diluents, distillates, or any combination thereof. In other embodiments, the distillates are selected from diesel, jet fuel, sustainable aviation fuel (SAF), or any combination thereof.

In one embodiment, ethylene oligomerization is utilized towards desirable products. In an embodiment, oligomerization of ethylene may be catalyzed by a homogeneous catalyst, heterogeneous catalyst, or any combination thereof and having transition metals as active sites. In some embodiments, ethylene is further converted into long chain hydrocarbons by oligomerization. In other embodiments, straight chain olefins are the main product from ethylene oligomerization. In some embodiments, alpha olefins are the main product from ethylene oligomerization. In an embodiment, olefins are subjected to upgrading processes. In some embodiments, the upgrading process of olefins is hydrogenation. In an embodiment, olefins are subjected to olefin conversion technology. In one embodiment, ethylene is interconverted to propylene, 2-butenes, or any combination thereof. In an embodiment, propylene is converted to polypropylene.

As a raw material, ethylene can used in the manufacture of polymers such as polyethylene (PE), polyethylene terephthalate (PET) and polyvinyl chloride (PVC), ethylene vinyl acetate (EVA), as well as fibres and other organic chemicals. These products are used in a wide variety of industrial and consumer markets such as the packaging, transportation, electrical/electronic, textile and construction industries as well as consumer chemicals, coatings and adhesives.

Ethylene can be chlorinated to ethylene dichloride (EDC) and can then be cracked to make vinyl chloride monomer (VCM). Nearly all VCM is used to make polyvinyl chloride which has its main applications in the construction industry.

Other ethylene derivatives include alpha olefins which are used in Linear low-density polyethylene (LLDPE) production, detergent alcohols and plasticizer alcohols; vinyl acetate monomer (VAM) which is used in adhesives, paints, paper coatings and barrier resins; and industrial ethanol which is used as a solvent or in the manufacture of chemical intermediates such as ethyl acetate and ethyl acrylate.

Ethylene may further be used as a monomer base for the production of various polyethylene oligomers by way of coordination polymerization using metal chloride or metal oxide catalysts. The most common catalysts consist of titanium (III) chloride, the so-called Ziegler-Natta catalysts. Another common catalyst is the Phillips catalyst, prepared by depositing chromium (VI) oxide on silica.

Polyethylene oligomers so produced may be classified according to its density and branching. Further, mechanical properties depend significantly on variables such as the extent and type of branching, the crystal structure, and the molecular weight. There are several types of polyethylene which may be generated from ethylene, including, but not limited to:
Ultra-high-molecular-weight polyethylene (UHMWPE);
Ultra-low-molecular-weight polyethylene (ULMWPE or PE-WAX);
High-molecular-weight polyethylene (HMWPE);
High-density polyethylene (HDPE);
High-density cross-linked polyethylene (HDXLPE);
Cross-linked polyethylene (PEX or XLPE);
Medium-density polyethylene (MDPE);
Linear low-density polyethylene (LLDPE);
Low-density polyethylene (LDPE);
Very-low-density polyethylene (VLDPE); and
Chlorinated polyethylene (CPE).

Low density polyethylene (LDPE) and linear low-density polyethylene (LLDPE) mainly go into film applications such as food and non-food packaging, shrink and stretch film, and non-packaging uses. High density polyethylene (HDPE) is used primarily in blow molding and injection molding applications such as containers, drums, household goods, caps and pallets. HDPE can also be extruded into pipes for water, gas and irrigation, and film for refuse sacks, carrier bags and industrial lining.

According to one embodiment, the ethylene formed from the disclosure described above may be converted to ethylene oxide via direct oxidation according to the following formula:

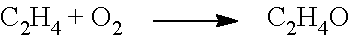

The ethylene oxide produced thereby is a key chemical intermediate in a number of commercially important processes including the manufacture of monoethylene glycol. Other EO derivatives include ethoxylates (for use in shampoo, kitchen cleaners, etc.), glycol ethers (solvents, fuels, etc.) and ethanolamines (surfactants, personal care products, etc.).

According to one embodiment of the disclosure, the ethylene oxide produced as described above may be used to produce commercial quantities of monoethylene glycol by way of the formula:

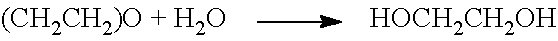

According to another embodiment, the claimed microorganism can be modified in order to directly produce monoethylene glycol. As described in WO 2019/126400, the disclosure of which is incorporated by reference in its entirety herein, the microorganism further comprises one or more of an enzymes capable of converting acetyl-CoA to pyruvate; an enzyme capable of converting pyruvate to oxaloacetate; an enzyme capable of converting pyruvate to malate; an enzyme capable of converting pyruvate to phosphoenolpyruvate; an enzyme capable of converting oxaloacetate to citryl-CoA; an enzyme capable of converting citryl-CoA to citrate; an enzyme capable of converting citrate to aconitate and aconitate to iso-citrate; an enzyme capable of converting phosphoenolpyruvate to oxaloacetate; an enzyme capable of converting phosphoenolpyruvate to 2-phospho-D-glycerate; an enzyme capable of converting 2-phospho-D-glycerate to 3-phospho-D-glycerate; an enzyme capable of converting 3-phospho-D-glycerate to 3-phosphonooxypyruvate; an enzyme capable of converting 3-phosphonooxypyruvate to 3-phospho-L-serine; an enzyme capable of converting 3-phospho-L-serine to serine; an enzyme capable of converting serine to glycine; an enzyme capable of converting 5,10-methylenetetrahydrofolate to glycine; an enzyme capable of converting serine to hydroxypyruvate; an enzyme capable of converting D-glycerate to hydroxypyruvate; an enzyme capable of converting malate to glyoxylate; an enzyme capable of converting glyoxylate to glycolate; an enzyme capable of converting hydroxypyruvate to glycolaldehyde; and/or an enzyme capable of converting glycolaldehyde to ethylene glycol.

In one embodiment, the microorganism comprises one or more of a heterologous enzyme capable of converting oxaloacetate to citrate; a heterologous enzyme capable of converting glycine to glyoxylate; a heterologous enzyme capable of converting iso-citrate to glyoxylate; a heterologous enzyme capable of converting glycolate to glycolaldehyde; or any combination thereof. In some embodiments, wherein the heterologous enzyme capable of converting oxaloacetate to citrate is a citrate [Si]-synthase [2.3.3.1], an ATP citrate synthase [2.3.3.8]; or a citrate (Re)-synthase [2.3.3.3]; the heterologous enzyme capable of converting glycine to glyoxylate is an alanine-glyoxylate transaminase [2.6.1.44], a serine-glyoxylate transaminase [2.6.1.45], a serine-pyruvate transaminase [2.6.1.51], a glycine-oxaloacetate transaminase [2.6.1.35], a glycine transaminase [2.6.1.4], a glycine dehydrogenase [1.4.1.10], an alanine dehydrogenase [1.4.1.1], or a glycine dehydrogenase [1.4.2.1]; the heterologous enzyme capable of converting iso-citrate to glyoxylate is an isocitrate lyase [4.1.3.1]; the heterologous enzyme capable of converting glycolate to glycolaldehyde is a glycolaldehyde dehydrogenase [1.2.1.21], a lactaldehyde dehydrogenase [1.2.1.22], a succinate-semialdehyde dehydrogenase [1.2.1.24], a 2,5-dioxovalerate dehydrogenase [1.2.1.26], an aldehyde dehydrogenase [1.2.1.3/4/5], a betaine-aldehyde dehydrogenase [1.2.1.8], or an aldehyde ferredoxin oxidoreductase [1.2.7.5]; or any combination thereof.

Monoethylene glycol produced according to either of the described methods may be used as a component of a variety of products including as a raw material to make polyester fibers for textile applications, including nonwovens, cover stock for diapers, building materials, construction materials, road-building fabrics, filters, fiberfill, felts, transportation upholstery, paper and tape reinforcement, tents, rope and cordage, sails, fish netting, seatbelts, laundry bags, synthetic artery replacements, carpets, rugs, apparel, sheets and pillowcases, towels, curtains, draperies, bed ticking, and blankets.

MEG may be used on its own as a liquid coolant, antifreeze, preservative, dehydrating agent, drilling fluid or any combination thereof. The MEG produced may also be used to produce secondary products such as polyester resins for use in insulation materials, polyester film, de-icing fluids, heat transfer fluids, automotive antifreeze and other liquid coolants, preservatives, dehydrating agents, drilling fluids, water-based adhesives, latex paints and asphalt emulsions, electrolytic capacitors, paper, and synthetic leather.

Importantly, the monoethylene glycol produced may be converted to the polyester resin polyethylene terephthalate (“PET”) according to one of two major processes. The first process comprises transesterification of the monoethylene glycol utilizing dimethyl terephthalate, according to the following two-step process:

### First Step

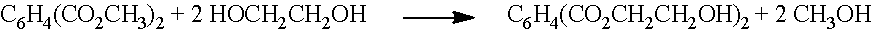

### Second Step

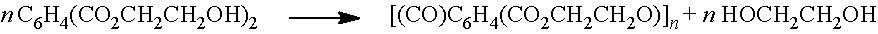

Alternatively, the monoethylene glycol can be the subject of an esterification reaction utilizing terephthalic acid according to the following reaction:

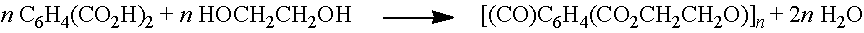

The polyethylene terephthalate produced according to either the transesterification or esterification of monoethylene glycol has significant applicability to numerous packaging applications such as jars and, in particular, in the production of bottles, including plastic bottles. It can also be used in the production of high-strength textile fibers such as Dacron, as part of durable-press blends with other fibers such as rayon, wool, and cotton, for fiber fillings used in insulated clothing, furniture, and pillows, in artificial silk, as carpet fiber, automobile tire yarns, conveyor belts and drive belts, reinforcement for fire and garden hoses, seat belts, nonwoven fabrics for stabilizing drainage ditches, culverts, and railroad beds, and nonwovens for use as diaper topsheets, and disposable medical garments.

At a higher molecular weight, PET can be made into a high-strength plastic that can be shaped by all the common methods employed with other thermoplastics. Magnetic recording tape and photographic film are produced by extrusion of PET film. Molten PET can be blow-molded into transparent containers of high strength and rigidity that are also virtually impermeable to gas and liquid. In this form, PET has become widely used in bottles, especially plastic bottles, and in jars.

The disclosure provides compositions comprising ethylene glycol produced by the microorganisms and according to the methods described herein. For example, the composition comprising ethylene glycol may be an antifreeze, preservative, dehydrating agent, or drilling fluid.

The disclosure also provides polymers comprising ethylene glycol produced by the microorganisms and according to the methods described herein. Such polymers may be, for example, homopolymers such as polyethylene glycol or copolymers such as polyethylene terephthalate. Methods for the synthesis of these polymers are well-known in the art. See, e.g., Herzberger et al., Chem Rev., 116(4): 2170-2243 (2016) and Xiao et al., Ind Eng Chem Res. 54(22): 5862-5869 (2015).

The disclosure further provides polyethylene glycol conjugates. In some embodiments, polyethylene glycol (PEG) conjugates include PEG conjugated to a biopharmaceutical, proteins, antibodies, anticancer drugs, or any combination thereof. In other embodiments, the PEG conjugate is diethyl terephthalate (DET). In some embodiments, the PEG conjugate is dimethoxyethane.

The disclosure further provides compositions comprising polymers comprising ethylene glycol produced by the microorganisms and according to the methods described herein. For example, the composition may be a fiber, resin, film, or plastic.

In one embodiment, ethanol or ethyl alcohol produced according to the method of the disclosure may be used in numerous product applications, including antiseptic hand rubs (WO 2014/100851), therapeutic treatments for methylene glycol and methanol poisoning (WO 2006/088491), as a pharmaceutical solvent for applications such as pain medication (WO 2011/034887) and oral hygiene products (U.S. Pat. No. 6,811,769), as well as an antimicrobial preservative (U.S. Patent Application No. 2013/0230609), engine fuel (U.S. Pat. No. 1,128,549), rocket fuel (U.S. Pat. No. 3,020,708), plastics, fuel cells (U.S. Pat. No. 2,405,986), home fireplace fuels (U.S. Pat. No. 4,692,168), as an industrial chemical precursor (U.S. Pat. No. 3,102,875), cannabis solvent (WO 2015/073854), as a winterization extraction solvent (WO 2017/161387), as a paint masking product (WO 1992/008555), as a paint or tincture (U.S. Pat. No. 1,408,091), purification and extraction of DNA and RNA (WO 1997/010331), and as a cooling bath for various chemical reactions (U.S. Pat. No. 2,099,090). In addition to the foregoing, the ethanol generated by the disclosed method may be used in any other application for which ethanol might otherwise be applicable.

In an additional embodiment, isopropanol or isopropyl alcohol (IPA) produced according to the method may be used in numerous product applications, including either in isolation or as a feedstock for the production for more complex products. Isopropanol may also be used in solvents for cosmetics and personal care products, de-icers, paints and resins, food, inks, adhesives, and pharmaceuticals, including products such as medicinal tablets as well as disinfectants, sterilisers and skin creams.

The IPA produced may be used in the extraction and purification of natural products such as vegetable and animal oil and fats. Other applications include its use as a cleaning and drying agent in the manufacture of electronic parts and metals, and as an aerosol solvent in medical and veterinary products. It can also be used as a coolant in beer manufacture, a coupling agent, a polymerisation modifier, a de-icing agent and a preservative.

Alternatively, the IPA produced according to the method of the disclosure may be used to manufacture additional useful compounds, including plastics, derivative ketones such as methyl isobutyl ketone (MIBK), isopropylamines and isopropyl esters. Still further, the IPA may be converted to propylene according to the following formula:

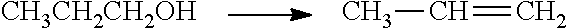

The propylene produced may be used as a monomer base for the production of various polypropylene oligomers by way of chain-growth polymerization via either gas-phase or bulk reactor systems. The most common catalysts consist of titanium (III) chloride, the so-called Ziegler-Natta catalysts and metallocene catalysts.

Polypropylene oligomers so produced may be classified according to tacticity and can be formed into numerous products by either extrusion or molding of polypropylene pellets, including piping products, heat-resistant articles such as kettles and food containers, disposable bottles (including plastic bottles), clear bags, flooring such as rugs and mats, ropes, adhesive stickers, as well as foam polypropylene which can be used in building materials. Polypropylene may also be used for hydrophilic clothing and medical dressings.

In an embodiment, the tandem repeat protein is squid ring teeth (SRT) protein. In an embodiment, the tandem repeat protein is an insect silk protein. In some embodiments, the tandem repeat protein is used in the manufacture of personal care products, textiles, plastics, biomedical products, or any combination thereof. In another embodiment, the tandem repeat protein comprises at least one polypeptide of the disclosure, a silk fiber and/or a copolymer of the disclosure, one or more acceptable carriers, or any combination thereof. In one embodiment, a product further comprises a drug. In another embodiment, a product is used as a medicine, in a medical device, a cosmetic, or any combination thereof. In an embodiment, the tandem repeat protein comprises a silk fiber, a copolymer, a drug, used for the manufacture of a medicament for treating or preventing a disease. In some embodiments, the tandem repeat proteins, fibers, copolymers, or any combination thereof can be used for a broad and diverse array of medical, military, industrial and commercial applications. In an embodiment, tandem repeat proteins can be used in the manufacture of medical devices comprising sutures, skin grafts, cellular growth matrices, replacement ligaments, surgical mesh, or any combination thereof. In other embodiments, the tandem repeat proteins can be used in industrial and commercial products comprising cable, rope, netting, fishing line, clothing fabric, bullet-proof vest lining, container fabric, backpacks, knapsacks, bag or purse straps, adhesive binding material, non-adhesive binding material, strapping material, tent fabric, tarpaulins, pool covers, vehicle covers, fencing material, sealant, construction material, weatherproofing material, flexible partition material, sports equipment, or any combination thereof. In an embodiment, the tandem repeat proteins can be used in any fiber or fabric for which high tensile strength and elasticity are desired. In an embodiment, the tandem repeat proteins may be used in a native form, a modified form, a derivative form, or any combination thereof. In some embodiments, the tandem repeat proteins can be spun together and/or bundled or braided with other fiber types. The present disclosure contemplates that the production of such combinations of the disclosure can be readily practiced to enhance any desired characteristics, including but not limited to appearance, softness, weight, durability, water-repellent properties, improved cost-of-manufacture, that may be generally sought in the manufacture and production of fibers for medical, industrial, or commercial applications. In some embodiments, the tandem repeat proteins are cosmetic and skin care compositions comprising anhydrous compositions having an effective amount of tandem repeat protein in a cosmetically acceptable medium. In an embodiment, the compositions include, but are not limited to, skin care, skin cleansing, make-up, anti-wrinkle products, or any combination thereof. In another embodiment, the composition comprises beauty soap, facial wash, shampoo, rinse, hair dye, hair cosmetics, general cream, emulsion, shaving cream, conditioner, cologne, shaving lotion, cosmetic oil, facial mask, foundation, eyebrow pencil, eye cream, eye shadow, mascara, perfume, tanning and sunscreen cosmetics, sunscreen lotion, nail cosmetics, eyeliner cosmetics, lip cosmetics, oral care products, toothpaste, or any combination thereof. In another embodiment, the tandem repeat protein is used in a coating on a bandage to promote wound healing, bandage material, a porous cloth, or any combination thereof. In an embodiment, the tandem repeat protein may be used in a film comprising a wound dressing material, an amorphous film, or any combination thereof. In one embodiment the tandem repeat protein is used in a stent, a stent graft, or any combination thereof. In an embodiment, the tandem repeat protein may be used in a thread, a braid, a sheet, a powder, or any combination thereof. In an embodiment, the stent graft may contain a coating on some or all of the tandem repeat protein, where the coating degrades upon insertion of the stent graft into a host, the coating thereby delaying contact between the tandem repeat protein and a host. Suitable coatings include, without limitation, gelatin, degradable polyesters (e.g., PLGA, PLA, MePEG-PLGA, PLGA-PEG-PLGA, and copolymers and blends thereof), cellulose and cellulose derivatives (e.g., hydroxypropyl cellulose), polysaccharides (e.g., hyaluronic acid, dextran, dextran sulfate, chitosan), lipids, fatty acids, sugar esters, nucleic acid esters, polyanhydrides, polyorthoesters and polyvinyl alcohol (PVA). In one embodiment, the tandem repeat protein containing stent grafts may contain a biologically active agent (drug), where the agent is released from the stent graft and then induces an enhanced cellular response (e.g., cellular or extracellular matrix deposition) and/or fibrotic response in a host into which the stent graft has been inserted. In some embodiments, the tandem repeat protein may also be used in a matrix for producing ligaments and tendons ex vivo. In an embodiment the tandem repeat protein is used in a hydrogel. In an embodiment, the tandem repeat proteins of the disclosure may be applied to the surface of fibers for use in textiles. In an embodiment, the fiber materials include, but are not limited to textile fibers of cotton, polyesters such as rayon and Lycra™, nylon, wool, and other natural fibers including native silk. In some embodiments, compositions suitable for applying the silk protein onto the fiber may include co-solvents such as ethanol, isopropanol, hexafluoranols, isothiocyanouranates, and other polar solvents that can be mixed with water to form solutions or microemulsions. The tandem repeat protein-containing solution may be sprayed onto the fiber or the fiber may be dipped into the solution. In some embodiments, flash drying of the coated material is utilized. In another embodiment, the tandem repeat protein composition is applied onto woven fibers. In one embodiment, the tandem repeat protein is used to coat stretchable weaves comprising stretchable clothing, stockings, or any combination thereof. In an embodiment, the tandem repeat protein can be added to polyurethane, other resins or thermoplastic fillers to prepare panel boards and other construction material or as moulded furniture and benchtops that replace wood and particle board. In an embodiment, the composites can also be used in building and automotive construction especially rooftops and door panels. In other embodiments, the tandem repeat proteins fibers re-enforce the resin making the material much stronger, including light weight construction which is of equal or superior strength to other particle boards and composite materials. In some embodiments, tandem repeat protein fibers are isolated and added to a synthetic composite-forming resin to be used in combination with plant-derived proteins, starch and oils to produce a biologically-based composite materials. In an embodiment, the tandem repeat protein is a paper additive. In another embodiment, the tandem repeat protein is used in technical and intelligent textiles. In some embodiments, the technical and intelligent textiles do not change properties when wet and maintain their strength and extensibility. In one embodiment, the tandem repeat proteins are used for functional clothing for sports and leisure wear, work wear, protective clothing, or any combination thereof. In some embodiments, the tandem repeat protein is used in clothing, equipment, materials for durability to prolonged exposure, heavy wear, personal protection from external environment, resistance to ballistic projectiles, resistant to fire and chemicals, or any combination thereof.

In one embodiment, ethylene is used to produce butadiene. In some embodiments the butadiene is used in rubber tires.

In an embodiment, a method for the continuous production of ethylene, the process comprising: passing a gaseous substrate to a bioreactor containing a culture of a recombinant C1-fixing microorganism capable of producing ethylene in a culture medium such that the microorganism converts the gaseous substrate to ethylene; and recovering the ethylene from the bioreactor.

In other embodiments, converting the ethylene into a component used to manufacture tires. In an embodiment, the ethylene is converted into a component used in tire threads.

The method according to an embodiment, wherein the tires are end-of-life tires.

The method according to an embodiment, wherein the gaseous substrate is derived from a process comprising tires.

The method according to an embodiment, wherein the gaseous substrate is derived from a product circularity process or a sustainable chemical process.

The method according to an embodiment, further comprising converting the ethylene to a component used to manufacture new tires.

The method according to an embodiment, comprising resin components selected from ethylene and other olefins bonded to synthetic components selected from butadiene and isoprene to form hybrid polymers used to manufacture tires.

One embodiment is directed to a method for producing a polymer from a gaseous substrate comprising a first gas fermentation process produces at least one first product selected from butadiene, isoprene, conjugated dienes, or any combination thereof and a second gas fermentation process produces at least one second product selected from ethylene and olefins, or any combination thereof, and wherein the at least one first product and at least one second product are copolymerized to form a polymer.

The method according to an embodiment, wherein the first gas fermentation process and the second gas fermentation process are run in parallel.

The method according to an embodiment, wherein the first gas fermentation process and the second gas fermentation process are both run continuously.

The method according to an embodiment, comprising a first gas fermentation process produces rubber component and a second gas fermentation process produces a resin component, and wherein the rubber component and resin component are copolymerized to form a polymer.

The method according to an embodiment, wherein the rubber component and resin component are copolymerized by a suitable polymerization catalyst.

The method according to an embodiment, wherein the rubber component is selected from butadiene, isoprene, conjugated dienes, or any combination thereof.

The method according to an embodiment, wherein the resin component is selected from ethylene, olefins, or any combination thereof.

The method according to an embodiment, wherein the suitable polymerization catalyst further comprises another component contained in a general polymerization catalyst composition containing a metallocene complex.

The method according to an embodiment, wherein the metallocene complex is a complex compound having one or more cyclopentadienyl groups or derivative cyclopentadienyl groups bonded to a central metal.

The method according to an embodiment, wherein the central metal is selected from a lanthanoid element, scandium, yttrium, or any combination thereof.

The method according to an embodiment, wherein the central metal is selected from samarium (Sm), neodymium (Nd), praseodymium (Pr), gadolinium (Gd), cerium (Ce), holmium (Ho), scandium (Sc), and yttrium (Y).

The method according to an embodiment, further comprising converting the polymer into a tire.

One embodiment for the circular production of tires from a gaseous substrate is directed to a first gas fermentation process to produce at least one first product selected from butadiene, isoprene, conjugated dienes, or any combination thereof; and a second gas fermentation process to produce at least one second product selected from ethylene and olefins, or any combination thereof, wherein the at least one first product and at least one second product are copolymerized to form a polymer, and wherein the substrate is derived from a process comprising tires.

The method according to an embodiment, wherein the substrate is derived from a process comprising end-of-life tires.

One embodiment is directed to a method for the circular production of tires, the method comprising: 1) passing a gaseous substrate to a first bioreactor containing a culture of a recombinant C1-fixing microorganism capable of producing at least one first product selected from butadiene, isoprene, conjugated dienes, or any combination thereof in a culture medium such that the microorganism converts the gaseous substrate to the at least one first product; and recovering the at least one first product from the bioreactor; 2) passing a gaseous substrate to a second bioreactor containing a culture of a recombinant C1-fixing microorganism capable of producing at least one second product selected from ethylene and olefins, or any combination thereof in a culture medium such that the microorganism converts the gaseous substrate to the at least one second product; and recovering the at least one second product from the bioreactor; 3) polymerizing the at least one first product with the at least one second product in the presence of a suitable polymerization catalyst to form a hybrid polymer; and 4) converting the hybrid polymer into a tire.

The method according to an embodiment, wherein the suitable polymerization catalyst further comprises another component contained in a general polymerization catalyst composition containing a metallocene complex.

The method according to an embodiment, wherein the metallocene complex is a complex compound having one or more cyclopentadienyl groups or derivative cyclopentadienyl groups bonded to a central metal.

The method according to an embodiment, wherein the central metal is selected from a lanthanoid element, scandium, yttrium, or any combination thereof.

The method according to an embodiment, wherein the central metal is selected from samarium (Sm), neodymium (Nd), praseodymium (Pr), gadolinium (Gd), cerium (Ce), holmium (Ho), scandium (Sc), and yttrium (Y).

The method according to an embodiment, wherein the first bioreactor and the second bioreactor are run in parallel.

The method according to an embodiment, wherein both the first bioreactor and the second bioreactor are continuously operated.

The method according to an embodiment, wherein the substrates are derived from a process comprising end-of-life tires.

The method according to an embodiment further comprising converting the isoprenoid into a product selected from synthetic rubber, block polymers containing styrene, thermoplastic rubbers, pressure-sensitive or thermosetting adhesives, butyl rubber, terpenes selected from citral, linalool, ionones, myrcene, L-menthol, N,N-diethylnerylamine, geraniol, nerolidols, flavours, fragrances, fuel additive, plastics, polyisoprene,

The method according to an embodiment further comprising converting the butadiene into a product selected from styrene-butadiene rubber, synthetic rubber, tires, component of tires, thermoplastic rubber, shoes, shoe soles, adhesives, sealants, asphalt, polymer modification components, nylon, ABS resins, chloroprene/neoprene rubber, nitrile rubber, plastics, acrylics, acrylonitrile-butadiene-styrene resins, and synthetic elastomers.

One embodiment is directed to a method for chemical recycling, the method comprising: a pyrolysis, gasification, and/or partial oxidation process; provided to a gas fermentation process; provided to a chemical product manufacturing process to produce a product comprising butadiene, isoprenoid, ethylene, polyethylene terephthalate (PET), or any combination thereof; provided to a synthetic rubber production process; provided to a tire manufacturing process; provided to a process of using tires; provided a process for the collecting and shredding of used tires; and provided back to the pyrolysis, gasification, and/or partial oxidation process.

One embodiment is directed to a method for chemical recycling, the method comprising: 1) a pyrolysis, gasification, and/or partial oxidation process; 2) provided to a gas fermentation process; 3) provided to a chemical product manufacturing process to produce a product comprising butadiene, isoprenoid, ethylene, polyethylene terephthalate (PET), or any combination thereof; 4) provided to a synthetic rubber production process; 5) provided to a tire manufacturing process; 6) provided to a process of using tires; 7) provided a process for the collecting and shredding of used tires; and 8) provided back to the pyrolysis, gasification, and/or partial oxidation process.

One embodiment is directed to a method for chemical recycling, the method comprising: 1) a pyrolysis, gasification, and/or partial oxidation process; 2) provided to a gas fermentation process; 3) provided to a chemical product manufacturing process to produce a commodity product; 4) provided to a synthetic rubber production process; 5) provided to a tire manufacturing process; 6) provided to a process of using tires; 7) provided a process for the collecting and shredding of used tires; and 8) provided back to the pyrolysis, gasification, and/or partial oxidation process.

Another embodiment is directed to a method for chemical recycling, the method comprising: 1) a pyrolysis, gasification, and/or partial oxidation process producing an effluent stream; 2) passing the effluent stream to a gas fermentation process to produce a product; 3) passing the gas fermentation product to a chemical product manufacturing process to produce a commodity product; 4) passing the commodity product to a synthetic rubber production process to produce synthetic rubber; 5) passing the synthetic rubber product to a tire manufacturing process to produce a tire; 6) providing the tire to a process of using tires; 7) passing the used tires to a process for the collecting and shredding of used tires; and 8) recycling used tires back to the pyrolysis, gasification, and/or partial oxidation process.

One embodiment is directed to a process for continuous co-production of at least one chemical product and at least one heterologous protein product comprising:
a) providing a continuous bioreactor;
b) introducing to the bioreactor a recombinant C1-fixing microorganism capable of co-producing at least one chemical product and at least one heterologous protein, a gaseous substrate comprising one or more of CO, CO₂, and H₂, and a liquid growth medium;
c) continuously culturing the recombinant C1-fixing microorganism thereby generating a gas fermentation broth comprising 1) the at least one chemical product, 2) the at least one heterologous protein product, and 3) microbial biomass;
d) continuously removing a portion of the gas fermentation broth in a first stream;
e) continuously removing the at least one chemical product in a second stream; and
f) continuously recovering the at least one heterologous protein from the microbial biomass from the first stream.

Another embodiment is directed to a method for the continuous co-production of at least one targeted chemical product and at least one heterologous protein product, the method comprising: a) culturing a recombinant C1-fixing microorganism capable of co-production of at least one targeted chemical product and at least one heterologous protein in the presence of a gaseous substrate comprising one or more of CO, CO₂, and H₂, wherein the culturing is a continuous fermentation process; and wherein the substrate and liquid nutrient medium of the culture are non-coalescing.

One embodiment is directed to a method for continuous co-production of at least one targeted chemical product and at least one heterologous protein product, the method comprising: a) culturing in a state of a continuous gas fermentation process, a recombinant C1-fixing microorganism capable of co-production of at least one targeted chemical product and at least one heterologous protein in a fermentation broth comprising the microorganism, a gaseous substrate comprising one or more of CO, CO₂, and H₂, and a liquid growth medium, wherein the fermentation broth comprises an equilibrium surface tension of from about 30 to about 40 mN/m.

Another embodiment is directed to a method for continuous co-production of at least one targeted chemical product and at least one heterologous protein product, the method comprising: a) culturing in a bioreactor, a recombinant C1-fixing microorganism capable of co-production of at least one targeted chemical product and at least one heterologous protein having a unit value in the presence of a gaseous substrate comprising one or more of CO, CO₂, and H₂, and a liquid growth medium; and recovering the at least one targeted chemical product and the at least one heterologous protein wherein the at least one heterologous protein is recovered in an amount from about 0.1% to about 1% grams/dry cell weight/day of the at least one heterologous protein produced.

The method of an embodiment, further comprising an initial stage of gas fermentation wherein the initial surface tension of the broth is from about 60 to about 72 mN/m.

The method of an embodiment, wherein the heterologous protein has a high market value.

The method of an embodiment, wherein the heterologous protein is a high-value, specialized protein.

The method of an embodiment, wherein the heterologous protein is an antioxidant enzyme.

The method of an embodiment, wherein the antioxidant enzyme is selected from catalase, glutathione peroxidase, vitamin C, vitamin E, beta-carotene, carotenoids, flavonoids, superoxide dismutase, or any combination thereof.

The method of an embodiment, wherein the antioxidant enzyme is superoxide dismutase.

The method of an embodiment, wherein the antioxidant enzyme is a superoxide dismutase selected from SOD006, SOD007, SOD009, and SOD010.

The method of an embodiment, wherein the at least one heterologous protein is squid ring teeth (SRT) protein and the at least one chemical product is ethylene.

The method of an embodiment, wherein the at least one chemical product is ethylene.

The method of an embodiment, further comprising separating the microbial biomass from the first stream before recovering the heterologous protein.

One embodiment is directed to a method for continuous co-production of at least one targeted chemical product and at least one exogenous protein product, the method comprising: a) culturing, in a bioreactor, a recombinant C1-fixing microorganism capable of co-production of at least one targeted chemical product and at least one heterologous protein in the presence of a gaseous substrate comprising one or more of CO, CO₂, and H₂, and a liquid growth medium; b) generating microbial biomass having a unit value, at least one targeted chemical product, and at least one heterologous protein having a unit value, wherein the unit value of the heterologous protein is greater than the unit value of the microbial biomass; and c) recovering the at least one heterologous protein in an amount of at least 15% of a sum value of the unit value of the heterologous protein and the unit value of the microbial biomass.

The method of an embodiment, wherein recovering of step c) of the at least one heterologous protein is in an amount of at least 1% of the sum value.

The method of an embodiment, wherein a protein or chemical is selected from bilirubin, glutathione, lipoic acid, N-acetyl cysteine, NADPH, NADH, ubiquinone, coenzyme Q10, uric acid, copper/zinc and manganese-dependent superoxide dismutase, iron-dependent catalase, selenium-dependent glutathione peroxidase, vitamin C, vitamin E, beta carotene, lycopene, lutein, flavonoids, flavones, flavonols, proanthocyanidins, albumin, ceruloplasmin, metallothionein, ferritin, myoglobin, transferrin, haptoglobins, ceruloplasmin, heat shock proteins, or any combination thereof.

The method of an embodiment, wherein the high-value, specialized protein is selected from ubiquinone, coenzyme Q10, copper/zinc and manganese-dependent superoxide dismutase, iron-dependent catalase, selenium-dependent glutathione peroxidase, albumin, ceruloplasmin, metallothionein, ferritin, myoglobin, transferrin, haptoglobins, ceruloplasmin, heat shock proteins, or any combination thereof.

The method of an embodiment, wherein the at least one chemical product is selected from 1-butanol, butyrate, butene, butadiene, methyl ethyl ketone, ethylene, acetone, isopropanol, lipids, 3-hydroxypropionate, terpenes, isoprene, fatty acids, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, monoethylene glycol, or any combination thereof.

The method of an embodiment, further comprising the recombinant microorganism comprising a disruptive mutation in one or more genes.

The method of an embodiment, wherein the recombinant microorganism comprises a parental microorganism selected from the group consisting of Cupriavidus necator.

The method of an embodiment, wherein the chemical product is one or more of ethylene, ethanol, acetone, isopropanol, or any combination thereof.

The method of an embodiment, further comprising a microbial biomass and at least one excipient.

The method of an embodiment, wherein the microbial biomass is suitable as animal feed.

The method of an embodiment, wherein the at least one heterologous protein is superoxide dismutase and the at least one chemical product is ethylene.

One embodiment is directed to a genetically engineered microorganism capable of producing a commodity chemical product, a tandem repeat protein product, microbial biomass, single cell protein (SCP), one or more intermediates, or any combination thereof.

In some aspects, the microbial biomass has a unit value. In one embodiment, the microbial biomass has a market value.

The microorganism according to an embodiment, wherein the microorganism produces a heterologous protein product, wherein the microorganism comprises a heterologous nucleic acid encoding at least one protein having tandem repeats.

The microorganism according to an embodiment, wherein the microorganism comprises a genetically engineered microorganism capable of co-producing at least one heterologous protein and at least one secreted chemical product from a gaseous substrate, the microorganism comprising a heterologous nucleic acid encoding the at least one protein having tandem repeats and a heterologous nucleic acid encoding the at least one secreted chemical product, wherein the microorganism is a C1-fixing bacteria.

The microorganism according to an embodiment, wherein the microorganism comprises a genetically engineered microorganism capable of co-producing at least one heterologous protein and at least one secreted chemical product from a gaseous substrate, the microorganism comprising a heterologous nucleic acid encoding the at least one protein having one or more tandem repeats and a heterologous nucleic acid encoding the at least one secreted chemical product, wherein the microorganism is a C1-fixing bacteria.

The microorganism according to an embodiment, wherein the microorganism comprises a genetically engineered C1-fixing microorganism capable of co-producing an heterologous protein and a chemical product from a gaseous substrate, the microorganism comprising a heterologous nucleic acid encoding the at least one heterologous protein having one or more tandem repeats and a heterologous nucleic acid encoding the at least one chemical product.

The microorganism according to an embodiment, wherein the microorganism comprises a genetically engineered C1-fixing microorganism capable of co-producing an heterologous protein and a chemical product from a gaseous substrate, the microorganism comprising a heterologous nucleic acid encoding the at least one heterologous protein having one or more tandem repeats and a heterologous nucleic acid encoding the at least one chemical product, wherein the microorganism is capable of accumulating the at least one heterologous protein in the cell and secreting the at least one chemical product from the cell.

The microorganism according to an embodiment, wherein the microorganism comprises a genetically engineered C1-fixing microorganism capable of co-producing at least one heterologous functional protein and at least one chemical product having two or more carbons from a gaseous substrate, the microorganism comprising a heterologous nucleic acid encoding at least one protein having tandem repeats and a heterologous nucleic acid encoding at least one secreted chemical product.

The microorganism according to an embodiment, wherein the microorganism comprises a genetically engineered C1-fixing microorganism capable of co-producing at least one heterologous functional protein and at least one chemical product having two or more carbons from a gaseous substrate, the microorganism comprising a heterologous nucleic acid encoding a group of genes comprising at least one protein having tandem repeats and at least one secreted chemical product.

The microorganism according to an embodiment, a genetically engineered microorganism capable of co-producing at least one heterologous protein and at least one chemical product from a gaseous substrate, the microorganism comprising a heterologous nucleic acid encoding the at least one protein having one or more tandem repeats and a heterologous nucleic acid encoding the at least one chemical product, wherein the microorganism is a C1-fixing bacteria.

The microorganism according to an embodiment, wherein the microorganism comprises a genetically engineered C1-fixing microorganism capable of co-producing at least one heterologous protein and at least one chemical product from a gaseous substrate, the microorganism comprising:
a) a heterologous nucleic acid encoding at least one heterologous protein having one or more tandem repeats; and
b) a heterologous nucleic acid encoding at least one chemical having two or more carbons, wherein the microorganism is capable of accumulating the at least one heterologous protein in the cell and secreting the at least one chemical product from the cell.

A method according to an embodiment, wherein the method of co-producing at least one heterologous protein and at least one chemical product by culturing the genetically engineered C1-fixing. microorganism in the presence of a gaseous substrate comprising one or more of CO, CO₂, and H₂, wherein the culturing is a continuous fermentation process.

A method according to an embodiment, the method of co-producing at least one heterologous protein having one or more tandem repeats and at least one chemical product by culturing the genetically engineered microorganism of claim 1 in the presence of a gaseous substrate comprising one or more of CO, CO₂, and H₂, wherein the culturing is a continuous fermentation process.

The method according to an embodiment, wherein the gaseous substrate comprises a C1-carbon source comprising one or more of CO, CO₂, and H₂.

The method according to an embodiment, wherein the gaseous substrate comprises syngas or industrial waste gas.

The method according to an embodiment, wherein the method of co-producing at least one heterologous protein having one or more tandem repeats and at least one chemical product by culturing the genetically engineered C1-fixing, wherein the chemical product is one or more of ethylene, ethanol, acetone, isopropanol, or any combination thereof.

The microorganism according to an embodiment, wherein the microorganism comprises a genetically engineered C1-fixing microorganism, wherein the at least one heterologous protein having one or more tandem repeats is selected from collagen, silk, elastin, keratin, resilin, titin, squid ring teeth (SRT) protein, suckerin, or any combination thereof.

The microorganism according to an embodiment, wherein the microorganism is a member of a genus selected from the group consisting of Cupriavidus.

The microorganism according to an embodiment, wherein the microorganism is derived from a parental bacterium selected from the group consisting of Cupriavidus necator.

The microorganism according to an embodiment, wherein the at least one heterologous protein having one or more tandem repeats is selected from silk or SRT protein.

The microorganism according to an embodiment, wherein the gas fermentation product is selected from an alcohol, an acid, a diacid, an alkene, a terpene, an isoprene, and alkyne, or any combination thereof.

The microorganism according to an embodiment, wherein the at least one secreted chemical product is selected from the group 1-butanol, butyrate, butene, butadiene, methyl ethyl ketone, ethylene, acetone, isopropanol, lipids, 3-hydroxypropionate, terpenes, isoprene, fatty acids, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, or monoethylene glycol.

The microorganism according to an embodiment, wherein the at least one secreted chemical product is selected from 1-butanol, butyrate, butene, butadiene, methyl ethyl ketone, ethylene, acetone, isopropanol, lipids, 3-hydroxypropionate, terpenes, isoprene, fatty acids, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, monoethylene glycol, or any combination thereof.

The microorganism according to an embodiment, wherein the microorganism further comprising a disruptive mutation in one or more genes.

The microorganism according to an embodiment, wherein the genetically engineered C1-fixing microorganism, further comprising a microbial biomass and at least one excipient.

The microorganism according to an embodiment, wherein the genetically engineered C1-fixing microorganism, wherein the microbial biomass is suitable as animal feed.

The microorganism according to an embodiment, wherein the genetically engineered C1-fixing microorganism, wherein the animal feed is suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The microorganism according to an embodiment, wherein the genetically engineered C1-fixing microorganism, wherein the microorganism is suitable as a single cell protein (SCP).

The microorganism according to an embodiment, wherein the genetically engineered C1-fixing microorganism, wherein the microorganism is suitable as a cell-free protein synthesis (CFPS) platform.

The microorganism according to an embodiment, wherein the genetically engineered C1-fixing microorganism, wherein the at least one secreted chemical product is native to the microorganism.

The microorganism according to an embodiment, wherein the genetically engineered microorganism of claim 1, wherein the at least one heterologous protein is squid ring teeth protein and the at least one chemical product is ethylene.

The microorganism according to an embodiment, wherein the at least one heterologous protein is silk protein and the at least one chemical product is ethylene.

The microorganism according to an embodiment, wherein the at least one chemical product is ethylene.

The method according to an embodiment, wherein the substrate comprises one or more of CO, CO₂, and H₂.

The method according to an embodiment, wherein at least a portion of the substrate is industrial waste gas, industrial off gas, or syngas.

The method according to an embodiment, wherein both anaerobic and aerobic gases can be used to feed separate cultures (e.g., an anaerobic culture and an aerobic culture) in two or more different bioreactors that are both integrated into the same process stream.

In some aspects of the microorganism disclosed herein, the microorganism is directed to a recombinant C1-fixing microorganism capable of producing C₆₋₁₈ fatty acids, C₆₋₁₈ fatty alcohols, or any combination thereof, from a gaseous substrate comprising a nucleic acid encoding a group of exogenous enzymes comprising thiolase; 3-hydroxyacyl-CoA dehydrogenase or 3-oxoacyl-CoA reductase; 3-hydroxyacyl-CoA dehydratase or enoyl-CoA hydratase; enoyl-CoA reductase; and one or more termination enzymes.

The microorganism of an embodiment, wherein the one or more termination enzymes is selected from thioesterase, phosphate acyltransferase, carboxylic acid kinase, or any combination thereof.

The microorganism of an embodiment, wherein the one or more termination enzymes is selected from alcohol forming acyl-CoA reductase, aldehyde forming acyl-CoA reductase and aldehyde reductase, thioesterase, carboxylic acid reductase and aldehyde reductase, phosphate acyltransferase and carboxylic acid kinase, carboxylic acid reductase, aldehyde reductase, or any combination thereof.

Another aspect of the microorganism is directed to A recombinant C1-fixing microorganism capable of producing C₆₋₁₈ fatty acids, C₆₋₁₈ fatty alcohols, or any combination thereof, from a gaseous substrate comprising a nucleic acid encoding a group of exogenous enzymes comprising: acetyl-CoA carboxylase; [acyl-carrier-protein]S-malonyltransferase; 3-oxoacyl-[acyl-carrier-protein]synthase I, II, and/or III; 3-oxoacyl-[acyl-carrier-protein]reductase; 3-hydroxyacyl-[acyl-carrier-protein]dehydratase; enoyl-[acyl-carrier-protein]reductase; and one or more termination enzymes.

The microorganism of an embodiment, wherein the one or more termination enzymes is acyl-ACP-thioesterase.

The microorganism of an embodiment, wherein the one or more termination enzymes is selected from alcohol forming acyl-ACP reductase, aldehyde forming acyl-ACP reductase, aldehyde reductase, acyl-ACP thioesterase, carboxylic acid reductase, aldehyde reductase, phosphate acyltransferase, carboxylic acid kinase, carboxylic acid reductase, aldehyde reductase, or any combination thereof.

The microorganism of an embodiment, wherein the microorganism is selected from the group consisting of Cupriavidus necator and Ralstonia eutropha.

The microorganism of an embodiment, wherein the microorganism is Cupriavidus necator.

The microorganism of an embodiment, further comprising an acyl-CoA primer and extender, wherein the primer and extender are capable of cyclic, iterative pathway operation.

The microorganism of an embodiment, further comprising one or more inducible promoters.

The microorganism of an embodiment, wherein the one or more inducible promoters is selected from an H₂ inducible promoter, a phosphate limited inducible promoter, a nitrogen limited inducible promoter, a CO₂ inducible promoter, or any combination thereof.

The microorganism of an embodiment, further comprising a disruptive mutation in one or more genes.

The microorganism of an embodiment, wherein the gaseous substrate comprises CO₂ and an energy source.

One embodiment is directed to a method for the continuous production of C₆₋₁₈ fatty acids and/or C₆₋₁₈ fatty alcohols, the process comprising: passing a gaseous substrate to a bioreactor containing a culture of a recombinant C1-fixing microorganism according to claim 1, in a culture medium such that the microorganism converts the gaseous substrate to C₆₋₁₈ fatty acids and/or C₆₋₁₈ fatty alcohols; and recovering the C₆₋₁₈ fatty acids and/or C₆₋₁₈ fatty alcohols from the bioreactor.

The microorganism of an embodiment, wherein the gaseous substrate comprises an industrial waste product or off-gas.

The microorganism of an embodiment, further comprising an energy source.

The microorganism of an embodiment, wherein the energy source is provided intermittently.

The microorganism of an embodiment, wherein the gaseous substrate comprises CO₂ and an energy source.

The microorganism of an embodiment, wherein the energy source is H₂.

The microorganism of an embodiment, wherein the gaseous substrate further comprises H₂, O₂, or both.

The microorganism of an embodiment, further comprising a step of limiting dissolved oxygen concentration, thereby switching a cellular burden.

Another embodiment is directed to a process for continuous co-production of at least one C₆₋₁₈ fatty acid or C₆₋₁₈ fatty alcohol and cultured protein comprising:
a) providing a continuous bioreactor;
b) introducing to the bioreactor a recombinant C1-fixing microorganism capable of co-producing at least one C₆₋₁₈ fatty acid or C₆₋₁₈ fatty alcohol, a gaseous substrate comprising one or more of CO, CO₂, and H₂, and a liquid growth medium;
c) continuously culturing the recombinant C1-fixing microorganism thereby generating a gas fermentation broth comprising 1) the at least one C₆₋₁₈ fatty acid or C₆₋₁₈ fatty alcohol, and 2) cultured protein;
d) continuously removing a portion of the gas fermentation broth in a first stream;
e) continuously removing the at least one C₆₋₁₈ fatty acid or C₆₋₁₈ fatty alcohol in a second stream; and continuously recovering the cultured protein from the first stream.

One embodiment is directed to a method for continuous co-production of at least one C₆₋₁₈ fatty acid or C₆₋₁₈ fatty alcohol and cultured protein, the method comprising: a) culturing, in a bioreactor, a recombinant C1-fixing microorganism capable of producing at least one C₆₋₁₈ fatty acid or C₆₋₁₈ fatty alcohol in the presence of a gaseous substrate comprising one or more of CO, CO₂, and H₂, and a liquid growth medium; b) generating cultured protein having a unit value, at least one C₆₋₁₈ fatty acid or C₆₋₁₈ fatty alcohol having a unit value, wherein the unit value of the cultured protein is greater than the unit value of the C₆₋₁₈ fatty acid or C₆₋₁₈ fatty alcohol; and c) recovering the cultured protein in an amount of at least 15% of a sum value of the unit value of the cultured protein and the unit value of the C₆₋₁₈ fatty acid or C₆₋₁₈ fatty alcohol.

The microorganism of an embodiment, wherein recovering of step c) of the cultured protein is in an amount of at least 1% of the sum value.

The microorganism of an embodiment, wherein the C₆₋₁₈ fatty acid is selected from hexanoic acid, octanoic acid, decanoic acid, dodecanoic acid, tetradecanoic acid, hexadecanoic acid, octadecanoic acid, palmitoleic acid, vaccenic acid, octadec-9-enoic acid, 8-(2-hexylcyclopropyl)octanoic acid, 8-(2-octylcyclopropyl)octanoic acid, 10-(2-hexylcyclopropyl)decanoic acid, 3-hydroxy-Cₙ-acid (i.e., 3-hydroxy-tetradecanoic acid), Cₙ-2-enoic acid (i.e., tetradec-2-enoic acid), or any combination thereof.

The microorganism of an embodiment, wherein the C₆₋₁₈ fatty acid is a branched-chain acid selected from anteiso-C13:0 (anteisotridecanoic acid), iso-C13:0 (isotridecanoic acid), anteiso-C14:0 (anteisotetradecanoic acid), iso-C14:0 (isotetradecanoic acid), anteiso-C15:0 (anteisopentadecanoic acid), iso-C15:0 (isopentadecanoic acid), anteiso-C16:0 (anteisohexadecanoic acid), iso-C16:0 (isohexadecanoic acid), anteiso-C17:0 (anteisoheptadecanoic acid), iso-C17:0 (isoheptadecanoic acid), anteiso-C18:0 (anteisooctadecanoic acid), iso-C18:0 (isooctadecanoic acid), anteiso-C19:0 (anteisononadecanoic acid), iso-C19:0 (isononadecanoic acid), or any combination thereof.

The microorganism of an embodiment, wherein the C₆₋₁₈ fatty alcohol is selected from hexanol, octanol, decanol, dodecanol, tetradecanol, hexadecanol, octadecanol, hexadec-9-en-1-ol, octadec-11-en-1-ol, Octadec-9-en-1-ol, 3-hydroxy-Cₙ-1-ol, Cₙ-2-en-1-ol, or any combination thereof.

The microorganism of an embodiment, wherein the C₆₋₁₈ fatty alcohol is a branched-chain alcohol selected from anteiso-C13:0 (anteisotridecanol), iso-C13:0 (isotridecanol), anteiso-C14:0 (anteisotetradecanol), iso-C14:0 (isotetradecanol), anteiso-C15:0 (anteisopentadecanol), iso-C15:0 (isopentadecanol), anteiso-C16:0 (anteisohexadecanol), iso-C16:0 (isohexadecanol), anteiso-C17:0 (anteisoheptadecanol), iso-C17:0 (isoheptadecanol), anteiso-C18:0 (anteisooctadecanol), iso-C18:0 (isooctadecanol), or any combination thereof.

The microorganism of an embodiment, further comprising the cultured protein and at least one excipient.

The microorganism of an embodiment, wherein the cultured protein is suitable as animal feed.

The microorganism of an embodiment, wherein the cultured protein is suitable as food.

### EXAMPLES

The following examples further illustrate the disclosure but, of course, should not be construed to limit its scope in any way.

### Example 1: Acyl-ACP:CoA Transacylase Mediated Acyl-ACP to Acyl-CoA Conversion in Cupriavidus necator Shifts Fatty Alcohol Production to Shorter Carbon Chain Length Compared to Acyl-ACP Reductase Mediated Production Autoethanogenum

Fatty alcohols can be produced utilizing the native fatty acid biosynthesis in Cupriavidus necator through the overexpression of an acyl-ACP reductase. However, production of C8-C12 carbon chain length products utilizing fatty acid biosynthesis requires highly active and specific enzymes (e.g., acyl-ACP reductases) to compete with FAB enzymes natively designed to further elongate these chain lengths. An alternative approach is the use of an acyl-ACP:CoA transacylase natively converting ACP intermediates to CoA intermediates to decouple product synthesis from the requirements of fatty acid biosynthesis for growth. To demonstrate this functionality in C. necator, fatty alcohol production via expression of the acyl-ACP reductase from Marinobacter sp. BSs20148 (MbACR) was compared to a route utilizing the Pseudomonas koreensis 3-hydroxyacyl-ACP:CoA transacylase (PkorPhaG).

The gene encoding the Marinobacter sp. BSs20148 acyl-ACP reductase (MbACR, UniProt: M1F7I4) was codon-adapted and synthesized for expression in Cupriavidus necator.

This gene was then cloned behind a synthetic nitrogen-limited inducible promoter in pBBR1MCS2. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

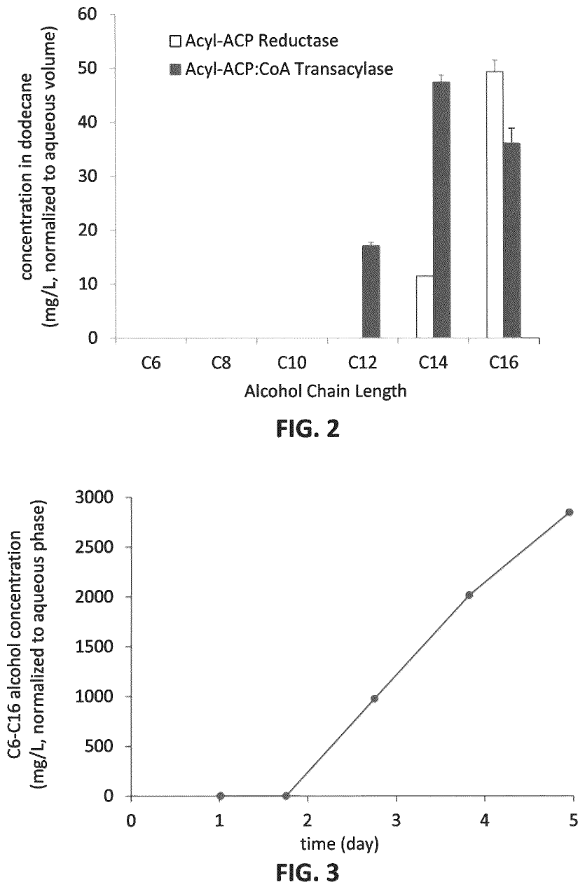

For acyl-ACP:CoA transacylase mediated production, genes encoding Pseudomonas koreensis 3-hydroxyacyl-ACP:CoA transacylase (PkorPhaG, WP_064586405.1), trans-enoyl-CoA reductase from Treponema denticola (TdTER, UniProt: Q73Q47), and Marinobacter hydrocarbonoclasticus VT8 acyl-CoA reductase (Maqu2507, WP_011785966.1) were codon-adapted and synthesized for expression in Cupriavidus necator. These genes were then cloned into pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs. For both strains, a single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH₄)₂SO₄ in a 125 mL Erlenmeyer Flask. 5 mL of dodecane was added to the flasks and the cultures were then grown at 30° C. and 200 rpm. Following 72 hrs of growth, the organic phase was collected and the concentration of fatty acids measured via GC-FID. As shown in FIG. 2, the use of the acyl-ACP:CoA transacylase mediated route for fatty alcohol production in C. necator resulted in a 65% increase in total C12-C16 titer and a 3.5-fold increase in C12/14 selectivity.

### Example 2: High-Titer Autotrophic Production C8-C14 of Fatty Alcohols Through Acyl-ACP:CoA Transacylase Mediated Acyl-ACP to Acyl-CoA Conversion in Cupriavidus necator

Genes encoding Pseudomonas koreensis 3-hydroxyacyl-ACP:CoA transacylase (PkorPhaG, WP_064586405.1), trans-enoyl-CoA reductase from Treponema denticola (TdTER, UniProt: Q73Q47), and Marinobacter hydrocarbonoclasticus VT8 acyl-CoA reductase (Maqu2507, WP_011785966.1) were codon-adapted and synthesized for expression in Cupriavidus necator. These genes were then cloned into pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

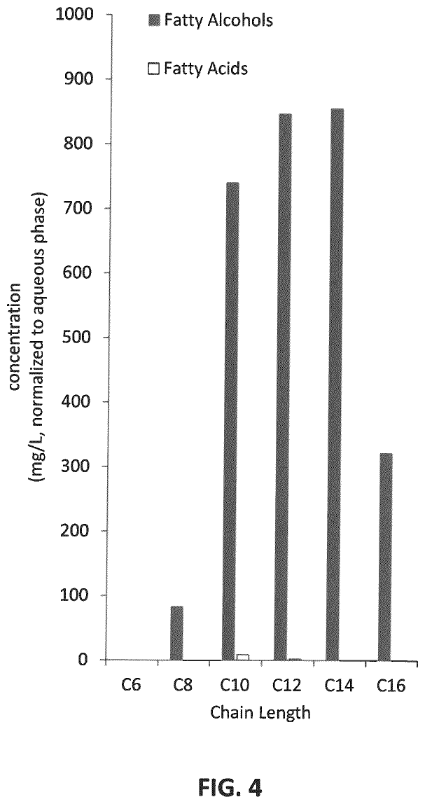

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3-0.4 was reached. 100 mL of the above culture was used to inoculate a 1.4-L Infors HT Multifors 2 CSTR containing 600 mL of media with 50 mg/L chloramphenicol. The reactor was incubated at 30° C. and initiated with 250 rpm agitation and 150 nccm gas flow (2.51-3.76% 02, 40% H2, 3% CO2, 54.49-53.24% N2). Agitation and gas flow were ramped up to 1250 rpm and 750 nccm as the culture grew. The base source was switched from NH₄OH to KOH to enable nitrogen limitation. 10% v/v dodecane was added to the reactor to capture the product. Samples from the reactor were taken periodically, the organic phase separated, and the concentration of fatty alcohols in the organic phase measured via GC-FID. As shown in FIG. 3, under autotrophic and nitrogen limited conditions this combination of enzymes utilizing a acyl-ACP:CoA transacylase resulted in nearly 3 g/L of total C8-C16 fatty alcohols with a chain length distribution centered around C12 (FIG. 4).

### Example 3: Improving Fatty Acid Production in Cupriavidus necator with Acyl-ACP:CoA Transacylase Expression

For acyl-ACP:CoA transacylase mediated production of fatty acids, genes encoding Pseudomonas koreensis 3-hydroxyacyl-ACP:CoA transacylase (PkorPhaG, WP_064586405.1), trans-enoyl-CoA reductase from Treponema denticola (TdTER, UniProt: Q73Q47), and E. coli thioesterase TesA′ (EcTesA′, UniProt: POADA1) were codon-adapted and synthesized for expression in Cupriavidus necator. These genes were then cloned into pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

To compare fatty acid production directly from acyl-ACP intermediates generated via fatty acid biosynthesis genes encoding trans-enoyl-CoA reductase from Treponema denticola (TdTER, UniProt: Q73Q47), and E. coli thioesterase TesA′ (EcTesA′, UniProt: POADA1) were codon-adapted and synthesized for expression in Cupriavidus necator. These genes were then cloned into pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

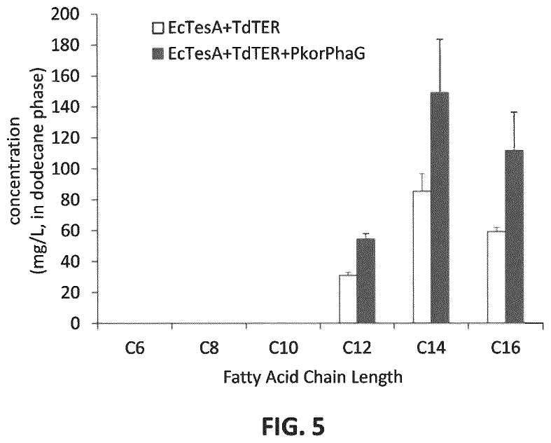

For both strains, a single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH₄)₂SO₄ in a 125 mL Erlenmeyer Flask. 5 mL of dodecane was added to the flasks and the cultures were then grown at 30° C. and 200 rpm. Following 72 hrs of growth, the organic phase was collected and the concentration of fatty acids measured via GC-FID. As shown in FIG. 5, the use of the acyl-ACP:CoA transacylase mediated route resulted in increased total C12-C16 fatty acid production in C. necator. As EcTesA′ is well-known to cleave both acyl-ACP and acyl-CoA intermediates this serves as a direct control between fatty acid production acyl-ACP cleavage only and production utilizing both acyl-ACP's (via FAB) and acyl-CoA's (generated via acyl-ACP:CoA transacylase).

### Example 4: Improving C10-C14 Fatty Acid Selectivity in Cupriavidus necator with Acyl-ACP:CoA Transacylase Expression

To demonstrate the ability for acyl-ACP:CoA transacylase mediated fatty acid production to improve C10-C14 fatty acid selectivity, the use of an acyl-CoA specific thioesterase was exploited (E. coli FadM).

For acyl-ACP:CoA transacylase mediated production of fatty acids, genes encoding Pseudomonas koreensis 3-hydroxyacyl-ACP:CoA transacylase (PkorPhaG, WP_064586405.1), trans-enoyl-CoA reductase from Treponema denticola (TdTER, UniProt: Q73Q47), and E. coli thioesterase FadM (EcFadM, UniProt: P77712) were codon-adapted and synthesized for expression in Cupriavidus necator. These genes were then cloned into pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

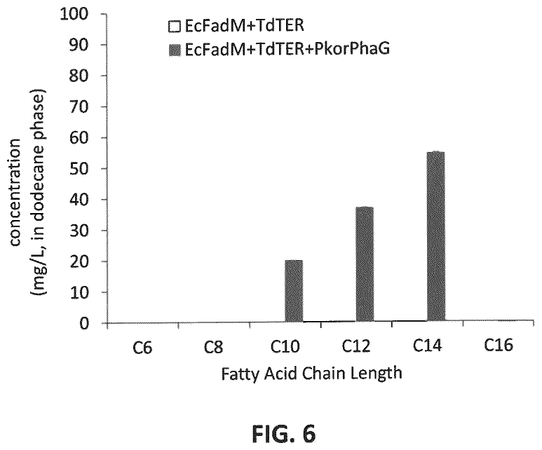

To compare fatty acid production directly from acyl-ACP intermediates generated via fatty acid biosynthesis genes encoding trans-enoyl-CoA reductase from Treponema denticola (TdTER, UniProt: Q73Q47), and E. coli thioesterase FadM (EcFadM, UniProt: P77712) were codon-adapted and synthesized for expression in Cupriavidus necator. These genes were then cloned into pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs. For both strains, a single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH₄)₂SO₄ in a 125 mL Erlenmeyer Flask. 5 mL of dodecane was added to the flasks and the cultures were then grown at 30° C. and 200 rpm. Following 72 hrs of growth, the organic phase was collected and the concentration of fatty acids measured via GC-FID. As shown in FIG. 6, fatty acids were only observed with acyl-ACP:CoA transacylase expression indicating production directly from acyl-CoA intermediates. Furthermore, this enabled selective production of C10-C14 fatty acids.

### Example 5: a System for Generating Bubbles within a Vessel

An example of a system of generating bubbles in a vessel 100 (FIG. 8). System 100 comprises cylindrical reactor 102. Liquid enters inlet or top portion 101 of reactor 102. The liquid may enter top portion 101 via an external pump in fluid communication with system 100. According to certain embodiments, the liquid entering top portion 101 is recirculated by an external pump in fluid communication with system 100. The liquid enters the top of perforated plate 104 and the liquid is accelerated by passing though the orifices in plate 104. According to certain examples, plate 104 may be configured to accelerate, for example, at least, greater than, less than, equal to, or any number from about 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99 to about 100% of the liquid in reactor 102. Sparger 106 injects gas bubbles into the liquid from gas source 108. Sparger 106 is positioned within reactor 102 such that a first zone is created in which the injected bubbles rise within reactor 102 and encounter accelerated liquid 112 exiting the bottom of plate 104. Accelerated liquid 112 from plate 104 breaks the rising bubbles into fine bubbles thereby increasing the superficial surface area required for the desired chemical or biological reaction. The fine bubbles may have a diameter in the range of about 0.1 mm to about 5 mm, or from about 0.5 mm to about 2 mm. In some examples, the fine bubbles may include a diameter from about 0.2 mm to 1.5 mm. According to another embodiment, the diameter of the fine bubbles may be, for example, at least, greater than, less than, equal to, or any number in between about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 to about 5.0 mm. Sparger 106 is further positioned within reactor 102 such that a second zone is created in which the fluid flow of liquid and fine bubbles may flow downward.

The fine bubbles may have a decreased rise velocity compared to the injected bubbles. Due to the overall flow of the accelerated liquid, fluid 116, containing the liquid and the fine bubbles, may have a net downward flow. The downward velocity of fluid 116 is greater than the overall rise velocity of the fine bubbles. Fluid 116 may exit reactor 102 at outlet 111. Plate 104 may have a thickness (and a depth of the orifices) from about 1 mm to 25 mm. According to another embodiment, the thickness of the plate may be, for example, at least, greater than, less than, equal to, or any number in between about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 mm.

The dimensions of the components of system 100, as illustrated in (FIG. 8), may vary depending upon the required use or process. According to certain embodiments, the diameter of the reactor 102 may be, for example, at least, greater than, less than, equal to, or any number in between about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5 to about 20.0 meters. According to other embodiments, the length of the reactor 102 may be, for example, at least, greater than, less than, equal to, or any number in between about 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.5, 21.5, 22.0, 22.5, 23.0, 23.5, 24.0, 24.5, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 to about 50.0 meters.

The velocity of the liquid or a portion of the liquid accelerated from plate 104 can be determined by the following equation:

QL=N×(π/4)×d2×vj
where QL is the liquid volumetric flow rate (m3/s), vj is the jet velocity, N is the total number of orifices on the plate, d is the diameter of the orifices, and π is the mathematical symbol pi. According to one embodiment, the velocity of the accelerated liquid from plate 104 may be, for example, at least, greater than, less than, equal to, or any number in between about 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500 to about 20000 mm/s. As depicted in FIG. 8, the velocity of accelerated liquid 112 is critical to breaking bubbles injected into the liquid by sparger 106 into properly sized fine bubbles, and to ensuring that the fluid of liquid and fine bubbles has enough velocity to generate a net downward fluid flow. The superficial liquid velocity, VL, in the main reaction vessel may be calculated by the following equation: VL=QL/AC where QL is the volumetric flow rate of the liquid (m3/s) in the reaction vessel and AC is the cross-sectional area of the reaction vessel. Therefore, superficial liquid velocity represents velocity of the liquid phase if it occupied the entire cross-sectional area of the reaction vessel. According to embodiments, the superficial liquid velocity may also include zones or voids of stagnant liquid and fine bubbles, and/or net downward fluid flow. For the same liquid flow rate, the gas flow rate can vary depending on the actual application. Superficial velocity of the gas phase VG may be determined by the following equation: VG=QG/AC where QG is the volumetric flow rate of the gas (m3/s) injected into the liquid from the sparger(s) and AC is the cross-sectional area of the reaction vessel. According to another embodiment, the superficial velocity of the gas phase in the vessel may be, for example, at least, greater than, less than, equal to, or any number in between about 5, 10, 15, 20, 25, 30, 35, 40, 45, 50, 55, 60, 65, 70, 75, 80, 85, 90, 95 to about 100 mm/s. According to still another embodiment, the superficial velocity of the gas phase in the vessel may be, for example, approximately 50-60 mm/s.

Positioning of a sparger or multiple spargers 106 within reactor 102, and in an upper portion of reactor 102 has the additional advantage of decreasing hydrostatic pressure at the top of reactor 102 facilitating increased gas to liquid mass transfer rates with decreased energy requirements. Further, required reactor components are minimized, yet gas to liquid mass transfer rates are maximized with a smaller reactor footprint due to decreased reactor size. In some embodiments, for example, the systems and methods disclosed herein achieve gas to liquid mass transfer rates of at least 125 m³/min. In other examples, the gas to liquid mass transfer rates may be, for example, at least, greater than, less than, equal to, or any number in between about 100, 105, 110, 115, 120, 125, 130, 135, 140, 145, 150, 155, 160, 165, 170, 175, 180, 185, 190, 195 to about 200 m³/min. Additionally, the sparger configurations, superficial velocities of the gas and liquid phases achieved, and the increased gas to liquid mass transfer rates disclosed herein overcome known obstacles associated with the use of a gas and liquid phase system of the previous and conventional reactors. Particularly in bioreactors having a gas substrate and an aqueous culture.

### Example 6: Production of Fatty Acids by Engineering a Beta-Oxidation Reversal in Cupriavidus necator

Genes encoding enzymes to produce fatty acids from acetyl-CoA were codon-adapted and synthesized for expression as operons in Cupriavidus necator. For production of C6-C10 fatty acids, genes encoding a native thiolase with up to C10 specificity (CnBktB, UniProt: Q0KBP1), a bifunctional beta-ketoacyl-CoA reductase/beta-hydroxyacyl-CoA dehydratase from E. coli (EcFadB, UniProt: C5A020), a trans-enoyl-CoA reductase from Treponema denticola (TdTER, UniProt: Q73Q47), and a thioesterase from E. coli (EcTesA′, UniProt: POADA1) known to have broad chain-length specificity and high activity (′ denotes truncated enzyme with periplasmic signal peptide removed) were organized as operons and cloned into the broad host range expression vector pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH₄)₂SO₄ in a 125 mL Erlenmeyer Flask. 5 mL of dodecane was added to the flasks and the cultures were then grown at 30° C. and 200 rpm. Following 24 hrs of growth, the aqueous and organic phases were collected and the concentration of fatty acids measured via GC-FID. The engineered Cupriavidus necator strain produced predominately C6-C10 fatty acids, with octanoic acid (C8) produced at the highest concentration.

### Example 7: Shifting Production to Shorter-Chain Length Fatty Acids Through an Engineered Beta-Oxidation Reversal in Cupriavidus necator

In order to produce predominately C6-C8 fatty acids, enzymes with higher specificity for shorter-chain length intermediates were selected for an engineered beta-oxidation reversal. This included the Clostridium kluyveri ThlA1 thiolase, the C. kluyveri HBD1 beta-ketoacyl-CoA reductase and C. acetobutylicum CRT crontase (beta-hydroxyacyl-CoA dehydratase). Genes encoding enzymes to produce fatty acids from acetyl-CoA were codon-adapted and synthesized for expression as operons in Cupriavidus necator. Genes encoding Clostridium kluyveri ThlA1 (UniProt: A5N3I5), C. kluyveri HBD1 (UniProt: A5N5D1), C. acetobutylicum CRT (UniProt: P52046), a trans-enoyl-CoA reductase from Treponema denticola (TdTER, UniProt: Q73Q47), and a thioesterase from E. coli (EcTesA′, UniProt: POADA1) were organized as operons and cloned into the broad host range expression vector pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH4)2SO4 in a 125 mL Erlenmeyer Flask. 5 mL of dodecane was added to the flasks and the cultures were then grown at 30° C. and 200 rpm. Following 24 hrs of growth, the aqueous and organic phases were collected and the concentration of fatty acids measured via GC-FID. The engineered Cupriavidus necator strain produced predominately C6-C8 fatty acids with this combination of rBOX enzymes.

### Example 8: Production of Fatty Alcohols by Engineering a Beta-Oxidation Reversal in Cupriavidus necator

In order to produce fatty alcohols opposed to fatty acids, an acyl-CoA reductase was utilized in combination with enzymes required for an engineered beta-oxidation reversal. Here, the Marinobacter hydrocarbonoclasticus VT8 acyl-CoA reductase (Maqu2507) that has broad chain length specificity (C6-C16 acyl-CoA's) was used. Genes encoding enzymes to produce fatty acids from acetyl-CoA were codon-adapted and synthesized for expression as operons in Cupriavidus necator. Genes encoding Clostridium kluyveri ThlA1 (UniProt: A5N3I5), C. kluyveri HBD1 (UniProt: A5N5D1), C. acetobutylicum CRT (UniProt: P52046), a trans-enoyl-CoA reductase from Treponema denticola (TdTER, UniProt: Q73Q47), and Maqu2507 (WP_011785966.1) were organized as operons and cloned into the broad host range expression vector pBBR1MCS2 behind synthetic nitrogen-limited inducible promoters. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH4)2SO4 in a 125 mL Erlenmeyer Flask. 5 mL of dodecane was added to the flasks and the cultures were then grown at 30° C. and 200 rpm. Following 48 hrs of growth, the aqueous and organic phases were collected and the concentration of fatty acids measured via GC-FID. The engineered Cupriavidus necator strain produced predominately C6-C8 fatty alcohols with this combination of rBOX enzymes and acyl-CoA reductase.

### Example 9: Production of Fatty Acids Through Fatty Acid Biosynthesis in Cupriavidus necator

Fatty acids can also be produced utilizing the native fatty acid biosynthesis in Cupriavidus necator through the overexpression of an acyl-ACP thioesterase. As the E. coli thioesterase TesA functions on both CoA and ACP intermediates, this enzyme was selected for fatty acid production via FAB. The gene encoding EcTesA′ (UniProt: POADA1, denotes truncated enzyme with periplasmic signal peptide removed) was codon-adapted and synthesized for expression in Cupriavidus necator. This gene was then cloned behind a rhamnose inducible promoter into pBBR1MCS2 modified for rhamnose inducible expression. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 25 mL J minimal media with 10 g/L fructose and 1 g/L (NH4)2SO4 in a 125 mL Erlenmeyer Flask. After 6 hours of growth, 0.1 mM rhamnose and 5 mL of dodecane was added to the flasks and the cultures grown at 30° C. and 200 rpm. Following 72 hrs of growth, the organic phase was collected and the concentration of fatty acids measured via GC-FID. The expression of EcTesA′ only in Cupriavidus necator PHB-4 resulted in the production of C12-C16 fatty acids.

### Example 10: Production of Fatty Alcohols Through Fatty Acid Biosynthesis in Cupriavidus necator

Fatty alcohols can also be produced utilizing the native fatty acid biosynthesis in Cupriavidus necator through the overexpression of an acyl-ACP reductase. Here, an acyl-ACP reductase from Marinobacter sp. BSs20148 (MbACR) was selected. The gene encoding MbACR (UniProt: M1F7I4) was codon-adapted and synthesized for expression in Cupriavidus necator. This gene was then cloned behind a phosphate-limited inducible promoter in pBBR1MCS2. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 1 mL modified J minimal media with 100 mM MOPS (pH 6.8) in place of the phosphate buffer with 10 g/L fructose, 10 g/L tryptone, 5 g/L yeast extract, 2.5 g/L (NH4)2SO4, and 5.16 mM PO4- in a deep-well 96-well plate and grown for 24 hrs at 1000 rpm and 30° C. This pre-culture was used to inoculate (1%) 25 mL of modified J minimal media (100 mM MOPS (pH 6.8) in place of the phosphate buffer) with 10 g/L fructose, 2.5 g/L (NH4)2SO4, and 0.516 mM PO4- in a 125 mL Erlenmeyer Flask. 5 mL of dodecane was added to the flasks and the cultures grown at 30° C. and 200 rpm. Following 72 hrs of growth, the organic phase was collected and the concentration of fatty acids measured via GC-FID. The expression of MbACR in Cupriavidus necator PHB-4 resulted in the production of C14-C16 fatty alcohols.

### Example 11: Autotrophic Production of Fatty Acids Through Fatty Acid Biosynthesis in Cupriavidus necator

The gene encoding EcTesA′ (UniProt: POADA1, ′ denotes truncated enzyme with periplasmic signal peptide removed) was codon-adapted and synthesized for expression in Cupriavidus necator. This gene was then cloned behind a rhamnose inducible promoter into pBBR1MCS2 modified for rhamnose inducible expression. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3-0.4 was reached.

100 mL of the above culture was used to inoculate a 1.4-L Infors HT Multifors 2 CSTR containing 600 mL of media with 50 mg/L chloramphenicol. The reactor was incubated at 30° C. and initiated with 250 rpm agitation and 150 nccm gas flow (2.51-3.76% 02, 40% H2, 3% CO2, 54.49-53.24% N2). Agitation and gas flow were ramped up to 1250 rpm and 750 nccm as the culture grew. Gene expression was induced by adding L-rhamnose to 0.1 mM in the reactor, and the base source was switched from NH40H to KOH to enable nitrogen limitation. 10% v/v dodecane was added to the reactor to capture the product. Samples from the reactor were taken periodically, the organic phase separated, and the concentration of fatty acids in the organic phase measured via GC-FID.

### Example 12: Autotrophic Production of Fatty Alcohols Through Fatty Acid Biosynthesis in Cupriavidus necator

The gene encoding the Marinobacter sp. BSs20148 acyl-ACP reductase (MbACR, UniProt: M1F7I4) was codon-adapted and synthesized for expression in Cupriavidus necator. This gene was then cloned behind a rhamnose inducible promoter into pBBR1MCS2 modified for rhamnose inducible expression. The resulting product was used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3-0.4 was reached.

100 mL of the above culture was used to inoculate a 1.4-L Infors HT Multifors 2 CSTR containing 600 mL of media with 50 mg/L chloramphenicol. The reactor was incubated at 30° C. and initiated with 250 rpm agitation and 150 nccm gas flow (2.51-3.76% 02, 40% H2, 3% CO2, 54.49-53.24% N2). Agitation and gas flow were ramped up to 1250 rpm and 750 nccm as the culture grew. Gene expression was induced by adding L-rhamnose to 0.1 mM in the reactor, and the base source was switched from NH4OH to KOH to enable nitrogen limitation. 10% v/v dodecane was added to the reactor to capture the product. Samples from the reactor were taken periodically, the organic phase separated, and the concentration of fatty alcohols in the organic phase measured via GC-FID.

TABLE 2
Representative Enzymes

| SEQ | | | | Uniprot/ | |
|---|---|---|---|---|---|
| ID | Enzyme | EC | Representative | GenBank | |
| NO | Type | Number(s) | Enzyme(s) | Accession | Sequence |
| 1 | 3- | 2.4.1.- | Pseudomonas | WP_ | [Nucleotide Sequence Used in |
| | hydroxy | | koreensis PhaG | 064586405.1 | Example] |
| | acyl- | | | | ATGCGCCCCGAAATTGCCGTGC |
| | ACP:CoA | | | | TCGACATCCAGGGCCAGTACCG |
| | transacy | | | | GGTCTATACCGAGTTCTACCGT |
| | lase | | | | GCCGATGCCGCGGAGAAGACC |
| | | | | | ATCATCCTGGTCAACGGCTCCA |
| | | | | | TGGCCACTACCGCCTCGTTCGC |
| | | | | | CCAGACCGTGAAGAACCTGCAT |
| | | | | | CCCCAGTTCAACGTCGTGTGCT |
| | | | | | ACGATCAGCCCTACGCTGGCCG |
| | | | | | CAGCAAGATCCACAACCGCCAT |
| | | | | | GAAAAGCATCTGACCAAGGAA |
| | | | | | GTCGAGGGCCAGATCCTGCTGG |
| | | | | | AGCTGATCGATCACTTCGGTGC |
| | | | | | CGAGCATGTGCTGTCGTTCAGC |
| | | | | | TGGGGCGGCGCGGCAACGCTGG |
| | | | | | TGGCGCTGTCGCACCAGCCCCG |
| | | | | | CCGCATCGAAAAGGCGGTGATC |
| | | | | | TCGTCCTTCAGCCCGGTGATCA |
| | | | | | ATGCGCACATGCTCGACTACCT |
| | | | | | GGAGCGCGGCGTTGACTACCTG |
| | | | | | GGCCAGCGCGACGGCGATCGTG |
| | | | | | TCGGCCATCTGGTCAACAATAC |
| | | | | | CATCGGCAAGCACCTACCGAGC |
| | | | | | CTGTTCAAACGCTTCAACTACC |
| | | | | | GTCACGTGTCCAGCCTGGCCGA |
| | | | | | GCATGAATACGGCCAGATGCAC |
| | | | | | TTCCATATCAACGATGTGCTCC |
| | | | | | ACAGCGATCGCCAGTGCTACCT |
| | | | | | GAACGCCGCCAAGAAGATCAA |
| | | | | | CGTGCCGGTGCTGTTTTTGAAC |
| | | | | | GGCGAGTGGGACGAATATACCG |
| | | | | | CCGCCGAGGATGCGCGCATCTT |
| | | | | | CGGCAACCACGTCGCCCAGTCG |
| | | | | | ACCTTTACCACGCTCCAGGCAA |
| | | | | | CCGGCCACTTCCTCGACATGGA |
| | | | | | GCACAAGGCCGCCTGCCGCGAC |
| | | | | | AGCCAGACCGCGCTGCTCGGCT |
| | | | | | TCCTCAAGCCGTCGCAACAGTC |
| | | | | | CAGCCGCACGCGCTACTCGTTC |
| | | | | | GTCCAGGATCACCATGCGCTGG |
| | | | | | CCATTTAA |
| 2 | 3- | 2.4.1.- | Pseudomonas | WP_ | [Amino Acid Sequence] |
| | hydroxy | | koreensis PhaG | 064586405.1 | MRPEIAVLDIQGQYRVYTEFYRA |
| | acyl- | | | | DAAEKTIILVNGSMATTASFAQT |
| | ACP:CoA | | | | VKNLHPQFNVVCYDQPYAGRSKI |
| | transacy | | | | HNRHEKHLTKEVEGQILLELIDHF |
| | lase | | | | GAEHVLSFSWGGAATLVALSHQ |
| | | | | | PRRIEKAVISSFSPVINAHMLDYL |
| | | | | | ERGVDYLGQRDGDRVGHLVNNT |
| | | | | | IGKHLPSLFKRFNYRHVSSLAEHE |
| | | | | | YGQMHFHINDVLHSDRQCYLNA |
| | | | | | AKKINVPVLFLNGEWDEYTAAE |
| | | | | | DARIFGNHVAQSTFTTLQATGHF |
| | | | | | LDMEHKAACRDSQTALLGFLKPS |
| | | | | | QQSSRTRYSFVQDHHALAI |
| 3 | 3- | 2.4.1.- | Pseudomonas | O85207 | [Amino Acid Sequence] |
| | hydroxy | | putida PhaG | | MRPEIAVLDIQGQYRVYTEFYRA |
| | acyl- | | | | DAAENTIILINGSLATTASFAQTV |
| | ACP:CoA | | | | RNLHPQFNVVLFDQPYSGKSKPH |
| | transacy | | | | NRQERLISKETEAHILLELIEHFQA |
| | lase | | | | DHVMSFSWGGASTLLALAHQPR |
| | | | | | YVKKAVVSSFSPVINEPMRDYLD |
| | | | | | RGCQYLAACDRYQVGNLVNDTI |
| | | | | | GKHLPSLFKRFNYRHVSSLDSHE |
| | | | | | YAQMHFHINQVLEHDLERALQG |
| | | | | | ARNINIPVLFINGERDEYTTVEDA |
| | | | | | RQFSKHVGRSQFSVIRDAGHFLD |
| | | | | | MENKTACENTRNVMLGFLKPTV |
| | | | | | REPRQRYQPVQQGQHAFAI |
| 4 | 3- | 2.4.1.- | Pseudomonas | Q93MS5 | [Amino Acid Sequence] |
| | hydroxy | | oleovorans | | MRPEIAVLDIQGQYRVYTEFYRA |
| | acyl- | | PhaG | | DAAENTIILINGSLATTASFAQTV |
| | ACP:CoA | | | | RNLHPQFNVVLFDQPYSGKSKPH |
| | transacy | | | | NRQERLISKETEAHILLELIEHFQA |
| | lase | | | | DHVMSFSWGGASTLLALAHQPR |
| | | | | | YVKKAVVSSFSPVINEPMRVYLD |
| | | | | | RGCQYLAACDHYQVGNLVNDTI |
| | | | | | GKHLPSLFKRFNYRHVSSLDSHE |
| | | | | | YAQMHFHINQVLEHDLERALQG |
| | | | | | ARNINIPVLFINGERDEYTTVEDA |
| | | | | | RQFSKHVGRSQFSVIRDAGHSLD |
| | | | | | MENKTACENTRNVMLGFLKPTV |
| | | | | | REPRQRYQPVQQGQHAFAI |
| 5 | (R)- | 4.2.1.119 | Pseudomonas | Q9HV20 | [Amino Acid Sequence] |
| | specific | | aeruginosa | | MPTAWLDLPAPPALPGLFLRAAL |
| | enoyl- | | PhaJ3 | | RRGIRGKALPERGLRSQVTVDPK |
| | CoA | | | | HLERYRQVCGFRDDGLLPPTYPH |
| | hydratase | | | | ILAFPLQMALLTDKRFPFPLLGLV |
| | | | | | HLENRIDVLRALGGLGPFTVSVA |
| | | | | | VENLQPHDKGATFSIVTRLEDQL |
| | | | | | GLLWVGDSKVLCRGVKVPGEIPP |
| | | | | | KAEQEPLPLEPVDNWKAPADIGR |
| | | | | | RYARAAGDYNPIHLSAPSAKLFG |
| | | | | | FPRAIAHGLWNKARSLAALGERL |
| | | | | | PASGYRVEVRFQKPVLLPASLTL |
| | | | | | LASAAAADGQFSLRGKDDLPHM |
| | | | | | AGHWSRLQG |
| 6 | Trans-2- | 1.3.1.44 | Treponema | Q73Q47 | [Nucleotide Sequence Used in |
| | enoyl- | | denticola FabV | | Example] |
| | CoA | | | | ATGATTGTCAAACCGATGGTGC |
| | reductase | | | | GCAACAATATCTGCCTCAATGC |
| | | | | | GCATCCGCAAGGCTGCAAGAAG |
| | | | | | GGCGTCGAGGATCAGATCGAGT |
| | | | | | ATACCAAGAAGCGCATTACCGC |
| | | | | | CGAGGTGAAGGCCGGTGCCAA |
| | | | | | GGCACCGAAGAACGTGCTGGTG |
| | | | | | CTGGGCTGCTCCAACGGCTACG |
| | | | | | GGCTGGCTTCCAGGATTACCGC |
| | | | | | GGCCTTCGGCTACGGTGCGGCG |
| | | | | | ACCATCGGCGTGAGCTTCGAGA |
| | | | | | AGGCAGGCTCGGAAACGAAGT |
| | | | | | ACGGCACGCCGGGCTGGTACAA |
| | | | | | CAACCTGGCCTTCGACGAGGCC |
| | | | | | GCCAAGCGCGAGGGGCTGTACA |
| | | | | | GCGTGACCATTGACGGCGACGC |
| | | | | | CTTCAGCGACGAGATCAAGGCG |
| | | | | | CAAGTGATCGAAGAGGCCAAG |
| | | | | | AAGAAGGGCATCAAGTTCGATC |
| | | | | | TGATCGTCTATTCGCTGGCCAG |
| | | | | | CCCGGTGCGTACCGATCCCGAT |
| | | | | | ACCGGCATCATGCACAAGAGCG |
| | | | | | TGCTGAAGCCGTTCGGCAAGAC |
| | | | | | CTTTACCGGCAAGACCGTCGAT |
| | | | | | CCGTTTACCGGCGAACTCAAGG |
| | | | | | AGATCTCGGCCGAGCCCGCCAA |
| | | | | | CGACGAAGAAGCCGCCGCGAC |
| | | | | | CGTCAAAGTGATGGGCGGCGAG |
| | | | | | GACTGGGAACGCTGGATCAAAC |
| | | | | | AGCTCTCCAAGGAAGGGCTGCT |
| | | | | | GGAAGAGGGCTGCATCACGCTG |
| | | | | | GCCTACTCGTACATCGGCCCCG |
| | | | | | AGGCGACCCAGGCGCTCTACCG |
| | | | | | CAAGGGTACCATCGGCAAGGCC |
| | | | | | AAGGAACATCTGGAAGCAACC |
| | | | | | GCGCATCGCCTGAACAAGGAGA |
| | | | | | ACCCGTCCATCCGTGCCTTCGT |
| | | | | | GTCGGTCAACAAGGGCCTGGTT |
| | | | | | ACCCGTGCCTCCGCCGTGATCC |
| | | | | | CGGTGATTCCGCTGTACCTGGC |
| | | | | | CTCGCTGTTCAAGGTCATGAAG |
| | | | | | GAGAAGGGCAACCACGAAGGC |
| | | | | | TGCATCGAACAGATCACGCGCC |
| | | | | | TGTACGCCGAACGCCTGTACCG |
| | | | | | CAAGGACGGGACCATCCCGGTC |
| | | | | | GACGAAGAGAACCGGATCCGC |
| | | | | | ATCGACGACTGGGAACTGGAAG |
| | | | | | AGGACGTCCAGAAGGCCGTGA |
| | | | | | GCGCACTGATGGAGAAGGTGAC |
| | | | | | CGGCGAGAATGCCGAATCGCTG |
| | | | | | ACCGATCTGGCGGGCTACCGCC |
| | | | | | ACGATTTCCTGGCCAGCAACGG |
| | | | | | CTTCGACGTGGAAGGCATCAAC |
| | | | | | TACGAGGCCGAAGTCGAGCGCT |
| | | | | | TCGATCGCATTTAA |
| 7 | Trans-2- | 1.3.1.44 | Treponema | Q73Q47 | [Amino Acid Sequence] |
| | enoyl- | | denticola FabV | | MIVKPMVRNNICLNAHPQGCKK |
| | CoA | | | | GVEDQIEYTKKRITAEVKAGAKA |
| | reductase | | | | PKNVLVLGCSNGYGLASRITAAF |
| | | | | | GYGAATIGVSFEKAGSETKYGTP |
| | | | | | GWYNNLAFDEAAKREGLYSVTI |
| | | | | | DGDAFSDEIKAQVIEEAKKKGIKF |
| | | | | | DLIVYSLASPVRTDPDTGIMHKS |
| | | | | | VLKPFGKTFTGKTVDPFTGELKEI |
| | | | | | SAEPANDEEAAATVKVMGGED |
| | | | | | WERWIKQLSKEGLLEEGCITLAY |
| | | | | | SYIGPEATQALYRKGTIGKAKEH |
| | | | | | LEATAHRLNKENPSIRAFVSVNK |
| | | | | | GLVTRASAVIPVIPLYLASLFKVM |
| | | | | | KEKGNHEGCIEQITRLYAERLYR |
| | | | | | KDGTIPVDEENRIRIDDWELEEDV |
| | | | | | QKAVSALMEKVTGENAESLTDL |
| | | | | | AGYRHDFLASNGFDVEGINYEAE |
| | | | | | VERFDRI |
| 8 | acyl- | 1.2.1.84; | Marinobacter | WP_ | [Nucleotide Sequence Used in |
| | CoA | 1.2.1.B25; | hydrocar- | 011785966.1 | Example] |
| | reductase | 1.2.1.42 | bonoclasticus | | ATGAACTATTTCCTGACTGGCG |
| | | | VT8 Maqu2507 | | GCACCGGCTTTATCGGCCGCTT |
| | | | | | CCTGGTCGAGAAGCTGCTCGCG |
| | | | | | CGCGGCGGCACGGTCTATGTGC |
| | | | | | TGGTGCGCGAACAAAGCCAGG |
| | | | | | ACAAGTTGGAGCGCCTGCGCGA |
| | | | | | GCGCTGGGGCGCCGACGACAA |
| | | | | | ACAGGTCAAGGCCGTGATCGGC |
| | | | | | GATCTGACCTCGAAGAACCTGG |
| | | | | | GCATCGACGCCAAGACGCTCAA |
| | | | | | GTCGCTGAAGGGCAACATCGAT |
| | | | | | CACGTGTTCCATCTGGCTGCCG |
| | | | | | TCTATGACATGGGCGCCGACGA |
| | | | | | GGAAGCCCAGGCCGCGACCAA |
| | | | | | CATCGAAGGTACCCGCGCCGCG |
| | | | | | GTCCAGGCCGCCGAGGCGATGG |
| | | | | | GCGCCAAGCACTTCCACCATGT |
| | | | | | CAGCAGCATTGCGGCCGCCGGC |
| | | | | | CTGTTCAAGGGCATCTTCCGCG |
| | | | | | AAGACATGTTCGAAGAAGCCGA |
| | | | | | GAAGCTGGATCACCCGTACCTG |
| | | | | | CGAACCAAGCACGAATCGGAA |
| | | | | | AAGGTGGTGCGCGAAGAATGC |
| | | | | | AAGGTGCCCTTCCGCATCTACA |
| | | | | | GGCCCGGCATGGTGATCGGTCA |
| | | | | | CAGCGAAACCGGCGAGATGGA |
| | | | | | CAAGGTGGACGGGCCCTACTAC |
| | | | | | TTCTTCAAGATGATCCAGAAGA |
| | | | | | TCCGCCATGCGCTGCCGCAATG |
| | | | | | GGTGCCGACCATCGGCATCGAG |
| | | | | | GGCGGCCGGCTCAATATCGTCC |
| | | | | | CGGTCGATTTCGTGGTCGACGC |
| | | | | | ACTGGACCATATCGCACATCTG |
| | | | | | GAAGGCGAGGACGGCAACTGC |
| | | | | | TTCCATCTGGTGGATTCTGATCC |
| | | | | | GTACAAGGTCGGCGAGATCCTG |
| | | | | | AACATCTTCTGCGAAGCCGGAC |
| | | | | | ACGCCCCGCGCATGGGCATGCG |
| | | | | | CATCGACAGCCGCATGTTCGGC |
| | | | | | TTTATCCCGCCGTTTATCCGGCA |
| | | | | | ATCGATCAAGAACCTGCCGCCG |
| | | | | | GTCAAGCGCATTACCGGCGCGC |
| | | | | | TGCTGGACGACATGGGCATCCC |
| | | | | | GCCCAGCGTGATGTCGTTCATC |
| | | | | | AACTACCCGACGCGCTTCGATA |
| | | | | | CCCGAGAGCTCGAGCGCGTCCT |
| | | | | | GAAGGGTACTGACATCGAGGTG |
| | | | | | CCGCGCCTGCCGAGCTACGCCC |
| | | | | | CGGTGATCTGGGACTACTGGGA |
| | | | | | GCGCAACCTGGACCCTGATCTG |
| | | | | | TTCAAGGACCGTACCCTGAAGG |
| | | | | | GAACCGTCGAGGGCAAGGTGTG |
| | | | | | CGTGGTGACCGGCGCAACGTCG |
| | | | | | GGCATCGGCCTGGCGACCGCCG |
| | | | | | AAAAGCTGGCCGAGGCCGGCG |
| | | | | | CGATCCTGGTGATCGGTGCCCG |
| | | | | | AACCAAGGAAACGCTCGACGA |
| | | | | | GGTGGCCGCCTCGCTCGAGGCC |
| | | | | | AAGGGCGGCAACGTGCACGCCT |
| | | | | | ACCAGTGCGACTTCAGCGACAT |
| | | | | | GGACGACTGCGACCGCTTCGTC |
| | | | | | AAGACCGTGCTGGACAACCATG |
| | | | | | GCCACGTCGACGTGCTGGTCAA |
| | | | | | TAATGCGGGCCGCAGCATCCGC |
| | | | | | CGCAGCCTGGCGCTGTCGTTCG |
| | | | | | ACCGCTTCCACGACTTCGAGCG |
| | | | | | CACGATGCAGCTCAACTACTTC |
| | | | | | GGCTCGGTGCGCCTGATCATGG |
| | | | | | GTTTTGCGCCCGCCATGCTTGA |
| | | | | | GCGCCGCCGCGGCCACGTGGTC |
| | | | | | AACATCAGCTCGATCGGCGTGC |
| | | | | | TGACCAACGCACCGCGCTTCAG |
| | | | | | CGCCTACGTGTCGTCGAAGAGC |
| | | | | | GCGCTGGACGCCTTCTCGCGCT |
| | | | | | GCGCGGCCGCCGAGTGGTCGGA |
| | | | | | CCGCAACGTGACGTTCACGACC |
| | | | | | ATCAACATGCCGCTGGTCAAGA |
| | | | | | CGCCGATGATCGCGCCGACCAA |
| | | | | | GATCTATGACTCCGTGCCGACG |
| | | | | | CTCACGCCGGATGAAGCCGCCC |
| | | | | | AGATGGTGGCCGATGCCATCGT |
| | | | | | CTACCGCCCGAAGCGCATTGCG |
| | | | | | ACCCGGCTTGGCGTCTTCGCAC |
| | | | | | AGGTGCTGCACGCGCTGGCCCC |
| | | | | | GAAGATGGGCGAAATCATCATG |
| | | | | | AATACCGGCTATCGCATGTTCC |
| | | | | | CGGATTCGCCGGCCGCCGCGGG |
| | | | | | CTCCAAGTCCGGCGAGAAGCCC |
| | | | | | AAGGTGAGCACGGAACAGGTG |
| | | | | | GCCTTCGCCGCCATCATGCGCG |
| | | | | | GCATCTACTGGTAA |
| 9 | acyl- | 1.2.1.84; | Marinobacter | WP_ | [Amino Acid Sequence] |
| | CoA | 1.2.1.B25; | hydrocar- | 011785966.1 | MNYFLTGGTGFIGRFLVEKLLAR |
| | reductase | 1.2.1.42 | bonoclasticus | | GGTVYVLVREQSQDKLERLRER |
| | | | VT8 Maqu2507 | | WGADDKQVKAVIGDLTSKNLGI |
| | | | | | DAKTLKSLKGNIDHVFHLAAVY |
| | | | | | DMGADEEAQAATNIEGTRAAVQ |
| | | | | | AAEAMGAKHFHHVSSIAAAGLF |
| | | | | | KGIFREDMFEEAEKLDHPYLRTK |
| | | | | | HESEKVVREECKVPFRIYRPGMV |
| | | | | | IGHSETGEMDKVDGPYYFFKMIQ |
| | | | | | KIRHALPQWVPTIGIEGGRLNIVP |
| | | | | | VDFVVDALDHIAHLEGEDGNCF |
| | | | | | HLVDSDPYKVGEILNIFCEAGHA |
| | | | | | PRMGMRIDSRMFGFIPPFIRQSIK |
| | | | | | NLPPVKRITGALLDDMGIPPSVM |
| | | | | | SFINYPTRFDTRELERVLKGTDIE |
| | | | | | VPRLPSYAPVIWDYWERNLDPDL |
| | | | | | FKDRTLKGTVEGKVCVVTGATS |
| | | | | | GIGLATAEKLAEAGAILVIGARTK |
| | | | | | ETLDEVAASLEAKGGNVHAYQC |
| | | | | | DFSDMDDCDRFVKTVLDNHGHV |
| | | | | | DVLVNNAGRSIRRSLALSFDRFH |
| | | | | | DFERTMQLNYFGSVRLIMGFAPA |
| | | | | | MLERRRGHVVNISSIGVLTNAPR |
| | | | | | FSAYVSSKSALDAFSRCAAAEWS |
| | | | | | DRNVTFTTINMPLVKTPMIAPTKI |
| | | | | | YDSVPTLTPDEAAQMVADAIVY |
| | | | | | RPKRIATRLGVFAQVLHALAPKM |
| | | | | | GEIIMNTGYRMFPDSPAAAGSKS |
| | | | | | GEKPKVSTEQVAFAAIMRGIYW |
| 10 | thio- | 3.1.2.2; | Escherichia | P0ADA1 | [Nucleotide Sequence Used in |
| | esterase | 3.1.2.20; | coli TesA | | Example] |
| | | 3.1.2.- | | | ATGGCCGATACGCTGCTGATCC |
| | | | | | TGGGTGATTCGCTGTCGGCCGG |
| | | | | | CTACCGCATGAGCGCCAGTGCC |
| | | | | | GCCTGGCCCGCGCTGCTCAACG |
| | | | | | ACAAATGGCAGTCCAAGACCAG |
| | | | | | CGTGGTCAATGCGTCGATCTCG |
| | | | | | GGCGATACCTCGCAACAGGGCC |
| | | | | | TGGCGCGCCTGCCCGCGCTCTT |
| | | | | | GAAACAGCACCAGCCGCGCTGG |
| | | | | | GTGCTGGTCGAGCTGGGCGGCA |
| | | | | | ACGACGGCCTGCGCGGCTTCCA |
| | | | | | GCCGCAACAGACCGAACAGAC |
| | | | | | GCTGCGCCAGATCCTCCAGGAT |
| | | | | | GTCAAGGCCGCCAATGCCGAAC |
| | | | | | CGCTGCTGATGCAGATCCGGCT |
| | | | | | GCCCGCCAACTACGGACGCCGC |
| | | | | | TACAACGAGGCCTTCTCGGCGA |
| | | | | | TCTACCCCAAGCTGGCGAAGGA |
| | | | | | GTTCGATGTGCCGCTGCTGCCC |
| | | | | | TTCTTCATGGAAGAGGTTTACC |
| | | | | | TCAAGCCCCAGTGGATGCAAGA |
| | | | | | CGACGGCATCCATCCGAACCGC |
| | | | | | GACGCCCAGCCATTTATCGCGG |
| | | | | | ACTGGATGGCAAAACAGCTCCA |
| | | | | | GCCGCTGGTCAACCACGATTCT |
| | | | | | TAA |
| 11 | thio- | 3.1.2.2; | Escherichia | P0ADA1 | [Amino Acid Sequence] |
| | esterase | 3.1.2.20; | coli TesA | | MADTLLILGDSLSAGYRMSASAA |
| | | 3.1.2.- | | | WPALLNDKWQSKTSVVNASISG |
| | | | | | DTSQQGLARLPALLKQHQPRWV |
| | | | | | LVELGGNDGLRGFQPQQTEQTLR |
| | | | | | QILQDVKAANAEPLLMQIRLPAN |
| | | | | | YGRRYNEAFSAIYPKLAKEFDVP |
| | | | | | LLPFFMEEVYLKPQWMQDDGIH |
| | | | | | PNRDAQPFIADWMAKQLQPLVN |
| | | | | | HDS |
| 12 | thio- | 3.1.2.2; | Escherichia | P77712 | [Nucleotide Sequence Used in |
| | esterase | 3.1.2.20; | coli FadM | | Example] |
| | | 3.1.2.- | | | ATGCAGACCCAGATAAAAGTGC |
| | | | | | GCGGCTACCATCTGGACGTCTA |
| | | | | | CCAGCATGTGAACAACGCGCGC |
| | | | | | TACCTGGAGTTCCTGGAAGAGG |
| | | | | | CGCGCTGGGACGGCCTGGAGAA |
| | | | | | CAGCGACAGCTTCCAATGGATG |
| | | | | | ACCGCCCACAACATCGCCTTTG |
| | | | | | TGGTGGTGAATATCAACATCAA |
| | | | | | CTACCGGCGCCCGGCCGTGTTG |
| | | | | | TCGGATCTGCTGACCATTACCA |
| | | | | | GCCAGCTGCAACAGCTGAACGG |
| | | | | | CAAGAGCGGCATCCTGAGCCAG |
| | | | | | GTGATCACGCTGGAGCCCGAGG |
| | | | | | GCCAGGTGGTGGCCGATGCGCT |
| | | | | | GATTACCTTCGTGTGCATCGAT |
| | | | | | CTGAAGACCCAGAAGGCGCTGG |
| | | | | | CGCTGGAAGGCGAGCTGCGCGA |
| | | | | | GAAGCTGGAACAGATGGTCAA |
| | | | | | ATAA |
| 13 | thio- | 3.1.2.2; | Escherichia | P77712 | [Amino Acid Sequence] |
| | esterase | 3.1.2.20; | coli FadM | | MQTQIKVRGYHLDVYQHVNNAR |
| | | 3.1.2.- | | | YLEFLEEARWDGLENSDSFQWM |
| | | | | | TAHNIAFVVVNININYRRPAVLSD |
| | | | | | LLTITSQLQQLNGKSGILSQVITLE |
| | | | | | PEGQVVADALITFVCIDLKTQKA |
| | | | | | LALEGELREKLEQMVK |

TABLE 3
Representative enzymes.

| SEQ | | | | |
|---|---|---|---|---|
| ID | | EC | | Uniprot/GenBank |
| NO. | Enzyme Type | Number(s) | Representative Enzyme(s) | Accession |
| 14 | thiolase | 2.3.1.9; | Cupriavidus necator BktB | Q0KBP1 |
| | | 2.3.1.16 | | |
| 15 | thiolase | 2.3.1.9; | Clostridium kluyveri ThlA1 | A5N3I5 |
| | | 2.3.1.16 | | |
| 16 | 3-hydroxyacyl-CoA | 1.1.1.35 | Escherichia coli FadB | C5A020 |
| | dehydrogenase or 3- | | | |
| | oxoacyl-CoA reductase | | | |
| 17 | 3-hydroxyacyl-CoA | 1.1.1.35 | Clostridium kluyveri HBD1 | A5N5D1 |
| | dehydrogenase or 3- | | | |
| | oxoacyl-CoA reductase | | | |
| 18 | 3-hydroxyacyl-CoA | 4.2.1.17; | Escherichia coli FadB | C5A020 |
| | dehydratase or enoyl- | 4.2.1.150 | | |
| | CoA hydratase | | | |
| 19 | 3-hydroxyacyl-CoA | 4.2.1.17; | Clostridium acetobutylicum | P52046 |
| | dehydratase or enoyl- | 4.2.1.150 | CRT | |
| | CoA hydratase | | | |
| 20 | Trans-2-enoyl-CoA | 1.3.1.44 | Treponema denticola FabV | Q73Q47 |
| | reductase | | | |
| 21 | acyl-CoA thioesterase | 3.1.2.2; | Escherichia coli TesA′ | P0ADA1 |
| | | 3.1.2.20; | | |
| | | 3.1.2.— | | |
| 22 | acyl-CoA thioesterase | 3.1.2.2; | Escherichia coli FadM | P77712 |
| | | 3.1.2.20; | | |
| | | 3.1.2.— | | |
| 23 | phosphate | 2.3.1.8; | Clostridium beijerinckii Ptb | Q05624 |
| | acyltransferase | 2.3.1.19; | | |
| | | 2.3.1.274 | | |
| 24 | carboxylic acid kinase | 2.7.2.7; | Clostridium felsineum Buk | WP_077892378.1 |
| | | 2.7.2.18; | | |
| | | 2.7.2.— | | |
| 25 | acyl-CoA reductase | 1.2.1.84; | Marinobacter | WP_011785966.1 |
| | (alcohol- or aldeyhde- | 1.2.1.B25; | hydrocarbonoclasticus VT8 | |
| | forming) | 1.2.1.42 | Maqu2507 | |
| 26 | aldehyde reductase or | 1.1.1.1; | Escherichia coli FucO | P0A9S1 |
| | alcohol dehydrogenase | 1.1.1.2; | | |
| 27 | aldehyde reductase or | 1.1.1.1; | Escherichia coli YjgB | P27250 |
| | alcohol dehydrogenase | 1.1.1.2; | | |
| 28 | carboxylic acid reductase | 1.2.1.— | Mycobacterium marinum CAR | B2HN69 |
| 29, | acetyl-CoA carboxylase | 6.4.1.2 | Cupriavidus necator AccA1, | Q0KCA7, |
| 30, | | | AccB, AccC2, AccD | Q0K6X5, |
| 31, | | | | Q0K6X4, |
| 32 | | | | Q0K8H8 |
| 33 | [acyl-carrier-protein] S- | 2.3.1.39 | Cupriavidus necator FabD | Q0K8M0 |
| | malonyltransferase; | | | |
| 34 | 3-oxoacyl-[acyl-carrier- | 2.3.1.41 | Cupriavidus necator FabB | Q0K0M2 |
| | protein] synthase I | | | |
| 35 | 3-oxoacyl-[acyl-carrier- | 2.3.1.179 | Cupriavidus necator FabF | Q0K8M3 |
| | protein] synthase II | | | |
| 36 | 3-oxoacyl-[acyl-carrier- | 2.3.1.180 | Cupriavidus necator FabH | Q0K8L9 |
| | protein] synthase III | | | |
| 37 | 3-oxoacyl-[acyl-carrier- | 1.1.1.100 | Cupriavidus necator FabG | Q0KDN0 |
| | protein] reductase | | | |
| 38 | 3-hydroxyacyl-[acyl- | 4.2.1.59 | Cupriavidus necator FabZ | Q0KA27 |
| | carrier-protein] | | | |
| | dehydratase | | | |
| 39 | enoyl-[acyl-carrier- | 1.3.1.9; | Cupriavidus necator FabI | Q0K919 |
| | protein] reductase | 1.3.1.10 | | |
| 40 | acyl-ACP-thioesterase | 3.1.2.21; | Umbellularia californica FatB1 | Q41635 |
| | | 3.1.2.— | | |
| 41 | acyl-ACP reductase | 1.2.1.80 | Marinobacter sp. BSs20148 | M1F7I4 |
| | | | Acr1 | |

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### EMBODIMENTS OF THE DISCLOSURE

Embodiment 1. A recombinant C1-fixing microorganism capable of producing C₆₋₁₈ fatty acids, C₆₋₁₈ fatty alcohols, or any combination thereof, from a gaseous substrate comprising a nucleic acid encoding a group of exogenous enzymes comprising a) thiolase having EC number 2.3.1.9 or 2.3.1.16; b) 3-hydroxyacyl-CoA dehydrogenase having EC number 1.1.1.35 or 3-oxoacyl-CoA reductase having EC number 1.1.1.330 or 2.3.1.199; c) 3-hydroxyacyl-CoA dehydratase having EC number 4.2.1.17 or enoyl-CoA hydratase having EC number 4.2.1.150; d) trans-2-enoyl-CoA reductase having EC number 1.3.1.93, 1.3.1.44, 1.3.1.37 or 1.3.1.38; and e) one or more termination enzymes.

Embodiment 2. The microorganism according to embodiment 1, wherein the one or more termination enzymes is selected from a) acyl-CoA thioesterase having EC number 3.1.2.20, b) phosphate acyltransferase having EC number 2.3.1.8, 2.3.1.19, or 2.3.1.274, c) carboxylic acid kinase having EC number 2.7.2.7, 2.7.2.18, or 2.7.2.-, or d) any combination thereof.

Embodiment 3. The microorganism according to any of embodiments 2 to 3, wherein the one or more termination enzymes is selected from a) alcohol forming acyl-CoA reductase, aldehyde forming acyl-CoA reductase having EC number 1.2.1.84, 1.2.1.B25, or 1.2.1.42, and aldehyde reductase having EC number 1.1.1.1 or 1.1.1.2, b) thioesterase having EC number 3.1.2.20, carboxylic acid reductase having EC number 1.2.1.- and aldehyde reductase having EC number 1.1.1.1 or 1.1.1.2, c) phosphate acyltransferase having EC number 2.3.1.18, 2.3.1.19, or 2.3.1.274 and carboxylic acid kinase having EC number 2.7.2.7, 2.7.2.18, or 2.7.2.-, carboxylic acid reductase having EC number 1.2.1.-, aldehyde reductase having EC number 1.2.1.84, or d) any combination thereof.

Embodiment 4. A recombinant C1-fixing microorganism capable of producing C₆₋₁₈ fatty acids, C₆₋₁₈ fatty alcohols, or any combination thereof, from a gaseous substrate comprising a nucleic acid encoding a group of exogenous enzymes comprising: a) acetyl-CoA carboxylase having EC number 6.4.1.2; b) [acyl-carrier-protein]S-malonyltransferase having EC number 2.3.1.39; c) 3-oxoacyl-[acyl-carrier-protein]synthase I having EC number 2.3.1.41, II having EC number 2.3.1.179, and/or III having EC number 2.3.1.180; d) 3-oxoacyl-[acyl-carrier-protein]reductase having EC number 1.1.1.100; e) 3-hydroxyacyl-[acyl-carrier-protein]dehydratase having EC number 4.2.1.59; f) enoyl-[acyl-carrier-protein]reductase having EC number 1.3.1.9 or 1.3.1.10; and g) one or more termination enzymes.

Embodiment 5. The microorganism according to embodiment 4, wherein the one or more termination enzymes is acyl-ACP-thioesterase having EC number 3.1.2.21 or 3.1.2.-.

Embodiment 6. The microorganism according to any of embodiments 4 to 5, wherein the one or more termination enzymes is selected from alcohol forming acyl-ACP reductase having EC number 1.2.1.80, aldehyde forming acyl-ACP reductase, alcohol dehydrogenase having EC number 1.1.1.2, acyl-ACP thioesterase having EC number 3.1.2.21 or 3.1.2.-, carboxylic acid reductase having EC number 1.2.1.-, aldehyde reductase having EC number 1.1.1.1 or 1.1.1.2, phosphate acyltransferase having EC number 2.3.1.1.8, 2.3.1.19, or 2.3.1.274, carboxylic acid kinase having EC number 2.7.2.7. 2.7.2.18, or 2.7.2.-, carboxylic acid reductase having EC number 1.2.1.-, or any combination thereof.

Embodiment 7. The microorganism according to any of embodiments 1 to 6, wherein the microorganism is selected from the group consisting of Cupriavidus necator and Ralstonia eutropha.

Embodiment 8. The microorganism according to embodiment 7, wherein the microorganism is Cupriavidus necator.

Embodiment 9. The microorganism according to any of embodiments 1 to 8, further comprising an acyl-CoA primer and extender, wherein the primer and extender are capable of cyclic, iterative pathway operation.

Embodiment 10. The microorganism according to any of embodiments 1 to 9, further comprising one or more inducible promoters.

Embodiment 11. The microorganism according to embodiment 10, wherein the one or more inducible promoters is selected from an H₂ inducible promoter, a phosphate limited inducible promoter, a nitrogen limited inducible promoter, a CO₂ inducible promoter, or any combination thereof.

Embodiment 12. The microorganism according to any of embodiments 1 to 11, further comprising a disruptive mutation in one or more genes.

Embodiment 13. The microorganism according to any of embodiments 1 to 12, wherein the gaseous substrate comprises CO₂ and an energy source.

Embodiment 14. A method for the continuous production of C₆₋₁₈ fatty acids and/or C₆₋₁₈ fatty alcohols, the process comprising: passing a gaseous substrate to a bioreactor containing a culture of a recombinant C1-fixing microorganism according to claim 1, in a culture medium such that the microorganism converts the gaseous substrate to C₆₋₁₈ fatty acids and/or C₆₋₁₈ fatty alcohols; and recovering the C₆₋₁₈ fatty acids and/or C₆₋₁₈ fatty alcohols from the bioreactor.

Embodiment 15. The microorganism according to any of embodiments 1 to 14, wherein the gaseous substrate comprises an industrial waste product or off-gas.

Embodiment 16. The microorganism according to any of embodiments 1 to 15, further comprising an energy source.

Embodiment 17. The microorganism according to embodiment 17, wherein the energy source is provided intermittently.

Embodiment 18. The microorganism according to any of embodiments 1 to 17, wherein the gaseous substrate comprises CO₂ and an energy source.

Embodiment 19. The microorganism according to embodiment 18, wherein the energy source is H₂.

Embodiment 20. The microorganism according to any of embodiments 1 to 19, wherein the gaseous substrate further comprises H₂, O₂, or both.

Embodiment 21. The microorganism according to any of embodiments 1 to 21, further comprising a step of limiting dissolved oxygen concentration, thereby switching a cellular burden.

Embodiment 22. A process for continuous co-production of at least one C₆₋₁₈ fatty acid or C₆₋₁₈ fatty alcohol and cultured protein comprising:
a) providing a continuous bioreactor;
b) introducing to the bioreactor a recombinant C1-fixing microorganism capable of co-producing at least one C₆₋₁₈ fatty acid or C₆₋₁₈ fatty alcohol, a gaseous substrate comprising one or more of CO, CO₂, and H₂, and a liquid growth medium;
c) continuously culturing the recombinant C1-fixing microorganism thereby generating a gas fermentation broth comprising 1) the at least one C₆₋₁₈ fatty acid or C₆₋₁₈ fatty alcohol, and 2) cultured protein;
d) continuously removing a portion of the gas fermentation broth in a first stream;
e) continuously removing the at least one C₆₋₁₈ fatty acid or C₆₋₁₈ fatty alcohol in a second stream; and continuously recovering the cultured protein from the first stream.

Embodiment 23. A method for continuous co-production of at least one C₆₋₁₈ fatty acid or C₆₋₁₈ fatty alcohol and cultured protein, the method comprising: a) culturing, in a bioreactor, a recombinant C1-fixing microorganism capable of producing at least one C₆₋₁₈ fatty acid or C₆₋₁₈ fatty alcohol in the presence of a gaseous substrate comprising one or more of CO, CO₂, and H₂, and a liquid growth medium; b) generating cultured protein having a unit value, at least one C₆₋₁₈ fatty acid or C₆₋₁₈ fatty alcohol having a unit value, wherein the unit value of the cultured protein is greater than the unit value of the C₆₋₁₈ fatty acid or C₆₋₁₈ fatty alcohol; and c) recovering the cultured protein in an amount of at least 15% of a sum value of the unit value of the cultured protein and the unit value of the C₆₋₁₈ fatty acid or C₆₋₁₈ fatty alcohol.

Embodiment 24. The microorganism according to embodiment 23, wherein recovering of step c) of the cultured protein is in an amount of at least 1% of the sum value.

Embodiment 25. The microorganism according to any of embodiments 23 to 24, wherein the C₆₋₁₈ fatty acid is selected from hexanoic acid, octanoic acid, decanoic acid, dodecanoic acid, tetradecanoic acid, hexadecanoic acid, octadecanoic acid, palmitoleic acid, vaccenic acid, octadec-9-enoic acid, 8-(2-hexylcyclopropyl)octanoic acid, 8-(2-octylcyclopropyl)octanoic acid, 10-(2-hexylcyclopropyl)decanoic acid, 3-hydroxy-Cₙ-acid (i.e., 3-hydroxy-tetradecanoic acid), Cₙ-2-enoic acid (i.e., tetradec-2-enoic acid), or any combination thereof.

Embodiment 26. The microorganism according to any of embodiments 23 to 25, wherein the C₆₋₁₈ fatty acid is a branched-chain acid selected from anteiso-C13:0 (anteisotridecanoic acid), iso-C13:0 (isotridecanoic acid), anteiso-C14:0 (anteisotetradecanoic acid), iso-C14:0 (isotetradecanoic acid), anteiso-C15:0 (anteisopentadecanoic acid), iso-C15:0 (isopentadecanoic acid), anteiso-C16:0 (anteisohexadecanoic acid), iso-C16:0 (isohexadecanoic acid), anteiso-C17:0 (anteisoheptadecanoic acid), iso-C17:0 (isoheptadecanoic acid), anteiso-C18:0 (anteisooctadecanoic acid), iso-C18:0 (isooctadecanoic acid), anteiso-C19:0 (anteisononadecanoic acid), iso-C19:0 (isononadecanoic acid), or any combination thereof.

Embodiment 27. The microorganism according to any of embodiments 1 to 26, wherein the C₆₋₁₈ fatty alcohol is selected from hexanol, octanol, decanol, dodecanol, tetradecanol, hexadecanol, octadecanol, hexadec-9-en-1-ol, octadec-11-en-1-ol, Octadec-9-en-1-ol, 3-hydroxy-Cₙ-1-ol, Cₙ-2-en-1-ol, or any combination thereof.

Embodiment 28. The microorganism according to any of embodiments 1 to 27, wherein the C₆₋₁₈ fatty alcohol is a branched-chain alcohol selected from anteiso-C13:0 (anteisotridecanol), iso-C13:0 (isotridecanol), anteiso-C14:0 (anteisotetradecanol), iso-C14:0 (isotetradecanol), anteiso-C15:0 (anteisopentadecanol), iso-C15:0 (isopentadecanol), anteiso-C16:0 (anteisohexadecanol), iso-C16:0 (isohexadecanol), anteiso-C17:0 (anteisoheptadecanol), iso-C17:0 (isoheptadecanol), anteiso-C18:0 (anteisooctadecanol), iso-C18:0 (isooctadecanol), or any combination thereof.

Embodiment 29. The microorganism according to embodiment 22, further comprising the cultured protein and at least one excipient.

Embodiment 30. The microorganism according to embodiment 22, wherein the cultured protein is suitable as animal feed.

Embodiment 31. The microorganism according to embodiment 22, wherein the cultured protein is suitable as food.

Embodiment 32. A recombinant C1-fixing microorganism capable of producing at least one C₆₋₁₈ fatty acid, at least one C₆₋₁₈ fatty alcohol, or any combination thereof, from a gaseous substrate comprising a nucleic acid encoding a group of exogenous enzymes comprising a) acetyl-CoA carboxylase having EC number 6.4.1.2; b) [acyl-carrier-protein]S-malonyltransferase having EC number 2.3.1.39; c) 3-oxoacyl-[acyl-carrier-protein]synthase I, II, or III having EC number 2.3.1.41, 2.3.1.179, or 2.3.1.180; d) 3-oxoacyl-[acyl-carrier-protein]reductase having EC number 1.1.1.100; e) 3-hydroxyacyl-[acyl-carrier-protein]:CoA transacylase having EC number 2.4.1.-; f) (R)-specific enoyl-CoA hydratase having EC number 4.2.1.119; g) trans-2-enoyl-CoA reductase having EC number 1.3.1.44; and h) one or more termination enzymes.

Embodiment 33. The microorganism according to embodiment 32, wherein the one or more termination enzymes is selected from a) thioesterase having EC number 3.1.2.20; b) phosphate acyltransferase having EC number 2.3.1.18, 2.3.1.19, or 2.3.1.274; c) carboxylic acid kinase having EC number 2.7.2.7, 2.7.2.18, or 2.7.2.-; or d) any combination thereof.

Embodiment 34. The microorganism according to any of embodiments 32-33, wherein the one or more termination enzymes is selected from a) alcohol forming acyl-CoA reductase, aldehyde forming acyl-CoA reductase and aldehyde reductase having EC number 1.2.1.84, 1.2.1.B25, or 1.2.1.42; b) thioesterase having EC number 3.1.2.20; c) carboxylic acid reductase having EC number 1.2.1.- and aldehyde reductase having EC number 1.2.1.84, 1.2.1.B25, or 1.2.1.42; d) phosphate acyltransferase having EC number 2.3.1.18, 2.3.1.19, or 2.3.1.274 and carboxylic acid kinase having EC number 2.7.2.7, 2.7.2.18, 2.7.2.-; e) carboxylic acid reductase having EC number 1.2.1.-; f) aldehyde reductase having EC number 1.2.1.84, g) or any combination thereof.

Embodiment 35. A process for continuous co-production of at least one C₆₋₁₈ fatty acid or C₆₋₁₈ fatty alcohol and cultured protein comprising:
a) providing a continuous bioreactor;
b) introducing to the bioreactor a recombinant C1-fixing microorganism according to claim 1, a gaseous substrate comprising one or more of CO, CO₂, and H₂, and a liquid growth medium;
c) continuously culturing the recombinant C1-fixing microorganism thereby generating a gas fermentation broth comprising 1) the at least one C₆₋₁₈ fatty acid or C₆₋₁₈ fatty alcohol, and 2) cultured protein;
d) continuously removing a portion of the gas fermentation broth in a first stream;
e) continuously removing the at least one C₆₋₁₈ fatty acid or C₆₋₁₈ fatty alcohol in a second stream; and continuously recovering the cultured protein from the first stream.

Embodiment 36. The microorganism according to any of embodiments 1 to 35, wherein the microorganism is selected from the group consisting of Cupriavidus necator and Ralstonia eutropha.

Embodiment 37. The microorganism according to any of embodiments 1 to 36, wherein the microorganism is Cupriavidus necator.

Embodiment 38. The microorganism according to any of embodiments 1 to 37, further comprising one or more inducible promoters.

Embodiment 39. A method for the continuous production of at least one C₆₋₁₈ fatty acid and/or at least one C₆₋₁₈ fatty alcohol, the process comprising: passing a gaseous substrate to a bioreactor containing a culture of a recombinant C1-fixing microorganism according to claim 1, in a culture medium such that the microorganism converts the gaseous substrate to at least one C₆₋₁₈ fatty acid and/or at least one C₆₋₁₈ fatty alcohol; and recovering the C₆₋₁₈ fatty acid and/or C₆₋₁₈ fatty alcohol from the bioreactor.

Embodiment 40. The method according to embodiment 39, wherein the microorganism further comprises one or more termination enzymes is selected from a) alcohol forming acyl-CoA reductase, aldehyde forming acyl-CoA reductase and aldehyde reductase having EC number 1.2.1.84, 1.2.1.B25, or 1.2.1.42; b) thioesterase having EC number 3.1.2.20; c) carboxylic acid reductase having EC number 1.2.1.- and aldehyde reductase having EC number 1.2.1.84, 1.2.1.B25, or 1.2.1.42; d) phosphate acyltransferase having EC number 2.3.1.18, 2.3.1.19, or 2.3.1.274 and carboxylic acid kinase having EC number 2.7.2.7, 2.7.2.18, 2.7.2.-; e) carboxylic acid reductase having EC number 1.2.1.-; f) aldehyde reductase having EC number 1.2.1.84, g) or any combination thereof.

Embodiment 41. The method according to any of embodiments 39 to 40, wherein the microorganism further comprises one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, and an acyl-CoA reductase.

Embodiment 42. The method according to any of embodiments 1 to 41, wherein the microorganism is selected from DSM 428, DSM 531, DSM 541, or DSM 34774.

Embodiment 43. The microorganism according to embodiment 32, wherein the 3-hydroxyacyl-[acyl-carrier-protein]:CoA transacylase is (R)-3-hydroxydecanoyl-ACP:CoA transacylase (PhaG).

1. A recombinant C1-fixing microorganism capable of producing at least one C₆₋₁₈ fatty acid, at least one C₆₋₁₈ fatty alcohol, or any combination thereof, from a gaseous substrate comprising a nucleic acid encoding a group of exogenous enzymes comprising a) acetyl-CoA carboxylase having EC number 6.4.1.2; b) [acyl-carrier-protein]S-malonyltransferase having EC number 2.3.1.39; c) 3-oxoacyl-[acyl-carrier-protein]synthase I, II, or III having EC number 2.3.1.41, 2.3.1.179, or 2.3.1.180; d) 3-oxoacyl-[acyl-carrier-protein]reductase having EC number 1.1.1.100; e) 3-hydroxyacyl-[acyl-carrier-protein]:CoA transacylase having EC number 2.4.1.-; f) (R)-specific enoyl-CoA hydratase having EC number 4.2.1.119; g) trans-2-enoyl-CoA reductase having EC number 1.3.1.44; and h) one or more termination enzymes.

2. The microorganism according to claim 1, wherein the one or more termination enzymes is selected from a) thioesterase having EC number 3.1.2.20; b) phosphate acyltransferase having EC number 2.3.1.18, 2.3.1.19, or 2.3.1.274; c) carboxylic acid kinase having EC number 2.7.2.7, 2.7.2.18, or 2.7.2.-; or d) any combination thereof.

3. The microorganism according to claim 1, wherein the one or more termination enzymes is selected from a) alcohol forming acyl-CoA reductase, aldehyde forming acyl-CoA reductase and aldehyde reductase having EC number 1.2.1.84, 1.2.1.B25, or 1.2.1.42; b) thioesterase having EC number 3.1.2.20; c) carboxylic acid reductase having EC number 1.2.1.- and aldehyde reductase having EC number 1.2.1.84, 1.2.1.B25, or 1.2.1.42; d) phosphate acyltransferase having EC number 2.3.1.18, 2.3.1.19, or 2.3.1.274 and carboxylic acid kinase having EC number 2.7.2.7, 2.7.2.18, 2.7.2.-; e) carboxylic acid reductase having EC number 1.2.1.-; f) aldehyde reductase having EC number 1.2.1.84, g) or any combination thereof.

4. A process for continuous co-production of at least one C₆₋₁₈ fatty acid or C₆₋₁₈ fatty alcohol and cultured protein comprising:
a) providing a continuous bioreactor;
b) introducing to the bioreactor a recombinant C1-fixing microorganism according to claim 1, a gaseous substrate comprising one or more of CO, CO₂, and H₂, and a liquid growth medium;
c) continuously culturing the recombinant C1-fixing microorganism thereby generating a gas fermentation broth comprising 1) the at least one C₆₋₁₈ fatty acid or C₆₋₁₈ fatty alcohol, and 2) cultured protein;
d) continuously removing a portion of the gas fermentation broth in a first stream;
e) continuously removing the at least one C₆₋₁₈ fatty acid or C₆₋₁₈ fatty alcohol in a second stream; and continuously recovering the cultured protein from the first stream.

5. The microorganism according to claim 1, wherein the microorganism is selected from the group consisting of Cupriavidus necator and Ralstonia eutropha.

6. The microorganism according to claim 1, wherein the microorganism is Cupriavidus necator.

7. The microorganism according to claim 1, further comprising one or more inducible promoters.

8. The microorganism according to claim 7, wherein the one or more inducible promoters is selected from an H₂ inducible promoter, a phosphate limited inducible promoter, a nitrogen limited inducible promoter, a CO₂ inducible promoter, or any combination thereof.

9. The microorganism according to claim 1, further comprising a disruptive mutation in one or more genes.

10. The microorganism according to claim 1, wherein the gaseous substrate comprises CO₂ and an energy source.

11. A method for the continuous production of at least one C₆₋₁₈ fatty acid and/or at least one C₆₋₁₈ fatty alcohol, the process comprising: passing a gaseous substrate to a bioreactor containing a culture of a recombinant C1-fixing microorganism according to claim 1, in a culture medium such that the microorganism converts the gaseous substrate to at least one C₆₋₁₈ fatty acid and/or at least one C₆₋₁₈ fatty alcohol; and recovering the C₆₋₁₈ fatty acid and/or C₆₋₁₈ fatty alcohol from the bioreactor.

12. The method according to claim 11, wherein the gaseous substrate comprises an industrial waste product or off-gas.

13. The method according to claim 11, further comprising an energy source.

14. The method according to claim 11, wherein the energy source is provided intermittently.

15. The method according to claim 11, wherein the gaseous substrate comprises CO₂ and an energy source.

16. The method according to claim 13, wherein the energy source is H₂.

17. The method according to claim 12, wherein the gaseous substrate further comprises H₂, O₂, or both.

18. The method according to claim 4, wherein the microorganism further comprises one or more termination enzymes is selected from a) alcohol forming acyl-CoA reductase, aldehyde forming acyl-CoA reductase and aldehyde reductase having EC number 1.2.1.84, 1.2.1.B25, or 1.2.1.42; b) thioesterase having EC number 3.1.2.20; c) carboxylic acid reductase having EC number 1.2.1.- and aldehyde reductase having EC number 1.2.1.84, 1.2.1.B25, or 1.2.1.42; d) phosphate acyltransferase having EC number 2.3.1.18, 2.3.1.19, or 2.3.1.274 and carboxylic acid kinase having EC number 2.7.2.7, 2.7.2.18, 2.7.2.-; e) carboxylic acid reductase having EC number 1.2.1.-; f) aldehyde reductase having EC number 1.2.1.84, g) or any combination thereof.

19. The method according to claim 4, wherein the microorganism further comprises one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, and an acyl-CoA reductase.

20. The method according to claim 4, wherein the microorganism is selected from DSM 428, DSM 531, DSM 541, or DSM 34774.
